(19) United States
(12) Patent Application Publication
(10) Pub. No.: US 2008/0188502 A1
(43) Pub. Date: Aug. 7, 2008

(54) New Compounds I

(75) Inventors: Jeremy Burrows, Sodertalje (SE); Fernando Huerta, Sodertalje (SE); Fredrik Lake, Sodertalje (SE); Torben Pedersen, Sodertalje (SE); Didier Rotticci, Sodertalje (SE); Tobias Rein, Sodertalje (SE); Karin Staaf, Sodertalje (SE); Ulrika Yngve, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 11/769,102

(22) Filed: Jun. 27, 2007

Related U.S. Application Data
(60) Provisional application No. 60/816,756, filed on Jun. 27, 2006.

(51) Int. Cl. A61K 31/506; C07D 401/04; A61P 25/28

(52) U.S. Cl. 514/275; 544/331

(57) ABSTRACT

The present invention relates to a compound of formula (I):

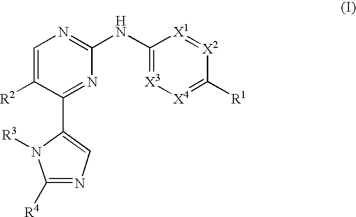

as a free base or a pharmaceutically acceptable salt thereof. The present invention also relates to pharmaceutical formulations containing said compound and to the use of said compound in therapy. The present invention further relates to a process for the preparation of compound of formula (I) and to new intermediates used therein.

### TECHNICAL FIELD OF INVENTION

The present invention relates to new compounds of formula (I), as a free base or a pharmaceutically acceptable salt thereof, to pharmaceutical formulations containing said compounds and to the use of said compounds in therapy. The present invention further relates to a process for the preparation of compounds of formula (I) and to new intermediates used therein.

### BACKGROUND OF THE INVENTION

Glycogen synthase kinase 3 (GSK3) is a serine/threonine protein kinase composed of two isoforms (α and β), which are encoded by distinct genes but are highly homologous within the catalytic domain. GSK3 is highly expressed in the central and peripheral nervous system. GSK3 phosphorylates several substrates including tau, β-catenin, glycogen synthase, pyruvate dehydrogenase and elongation initiation factor 2b (eIF2b). Insulin and growth factors activate protein kinase B, which phosphorylates GSK3 on serine 9 residue and inactivates it.

### Alzheimer's Disease (AD) Dementias, and Taupathies.

AD is characterized by cognitive decline, cholinergic dysfunction and neuronal death, neurofibrillary tangles and senile plaques consisting of amyloid-β deposits. The sequence of these events in AD is unclear, but is believed to be related. Glycogen synthase kinase 3β (GSK3β) or Tau phosphorylating kinase selectively phosphorylates the microtubule associated protein Tau in neurons at sites that are hyperphosphorylated in AD brains.

Hyperphosphorylated tau has lower affinity for microtubules and accumulates as paired helical filaments, which are the main components that constitute neurofibrillary tangles and neuropil threads in AD brains. This results in depolymerization of microtubules, which leads to dying back of axons and neuritic dystrophy. Neurofibrillary tangles are consistently found in diseases such as AD, amyotrophic lateral sclerosis, parkinsonism-dementia of Gaum, corticobasal degeneration, dementia pugilistica and head trauma, Down's syndrome, postencephalatic parkinsonism, progressive supranuclear palsy, Niemann-Pick's Disease and Pick's Disease. Addition of amyloid-β to primary hippocampal cultures results in hyperphosphorylation of tau and a paired helical filaments-like state via induction of GSK3β activity, followed by disruption of axonal transport and neuronal death (Imahori and Uchida, J. Biochem. 1997, 121:179-188). GSK3β preferentially labels neurofibrillary tangles and has been shown to be active in pre-tangle neurons in AD brains. GSK3 protein levels are also increased by 50% in brain tissue from AD patients. Furthermore, GSK3β phosphorylates pyruvate dehydrogenase, a key enzyme in the glycolytic pathway and prevents the conversion of pyruvate to acetyl-Co-A (Hoshi et al., PNAS 1996, 93: 2719-2723). Acetyl-Co-A is critical for the synthesis of acetylcholine, a neurotransmitter with cognitive functions. Accumulation of amyloid-β is an early event in AD. GSK Tg mice show increased levels of amyloid-β in brain. Also, PDAPP mice fed with Lithium show decreased amyloid-β levels in hippocampus and decreased amyloid plaque area (Su et al., Biochemistry 2004, 43: 6899-6908). Thus, GSK3β inhibition may have beneficial effects in progression as well as the cognitive deficits associated with Alzheimer's disease and other above-referred to diseases.

### Chronic and Acute Neurodegenerative Diseases

Growth factor mediated activation of the PI3K/Akt pathway has been shown to play a key role in neuronal survival. The activation of this pathway results in GSK3β inhibition. Recent studies (Bhat et. al., PNAS 2000, 97: 11074-11079) indicate that GSK3β activity is increased in cellular and animal models of neurodegeneration such as cerebral ischemia or after growth factor deprivation. For example, the active site phosphorylation was increased in neurons vulnerable to apoptosis, a type of cell death commonly thought to occur in chronic and acute degenerative diseases such as cognitive disorders, Alzheimer's Disease, Parkinson's Disease, amyotrophic lateral sclerosis, Huntington's Disease and HIV dementia and traumatic brain injury; and as in ischemic stroke. Lithium was neuroprotective in inhibiting apoptosis in cells and in the brain at doses that resulted in the inhibition of GSK3β. Thus GSK3β inhibitors could be useful in attenuating the course of neurodegenerative diseases.

### Bipolar Disorders (BD)

Bipolar Disorders are characterised by manic episodes and depressive episodes. Lithium has been used to treat BD based on its mood stabilising effects. The disadvantage of lithium is the narrow therapeutic window and the danger of overdosing that can lead to lithium intoxication. The discovery that lithium inhibits GSK3 at therapeutic concentrations has raised the possibility that this enzyme represents a key target of lithium's action in the brain (Stambolic et al., Curr. Biol. 1996, 68(12):1664-1668, 1996; Klein and Melton; PNAS 1996, 93:8455-8459; Gould et al., Neuropsychopharmacology, 2005, 30:1223-1237). GSK3 inhibitor has been shown to reduce immobilisation time in forced swim test, a model to assess on depressive behavior (O'Brien et al., J Neurosci 2004, 24(30): 6791-6798). GSK3 has been associated with a polymorphism found in bipolar II disorder (Szczepankiewicz et al., Neuropsychobiology. 2006, 53: 51-56). Inhibition of GSK3β may therefore be of therapeutic relevance in the treatment of BD as well as in AD patients that have affective disorders.

### Schizophrenia

Accumulating evidence implicates abnormal activity of GSK3 in mood disorders and schizophrenia. GSK3 is involved in signal transduction cascades of multiple cellular processes, particularly during neural development. (Kozlovsky et al., Am. J. Psychiatry, 2000, 157, 5: 831-833) found that GSK3β levels were 41% lower in the schizophrenic patients than in comparison subjects. This study indicates that schizophrenia involves neurodevelopmental pathology and that abnormal GSK3 regulation could play a role in schizophrenia. Furthermore, reduced β-catenin levels have been reported in patients exhibiting schizophrenia (Cotter et al., Neuroreport 1998, 9(7):1379-1383). Atypical antipsychotic such as olanzapine, clozapine, quetiapine, and ziprasidone, inhibits GSK3 by increasing ser9 phosphorylation suggesting that antipsychotics may exert their beneficial effects via GSK3 inhibition (Li X. et al., Int. J. of Neuropsychopharmacol, 2007, 10: 7-19, Epubl. 2006, May 4).

### Diabetes

Insulin stimulates glycogen synthesis in skeletal muscles via the dephosphorylation and thus activation of glycogen synthase. Under resting conditions, GSK3 phosphorylates and inactivates glycogen synthase via dephosphorylation. GSK3 is also over-expressed in muscles from Type II diabetic patients (Nikoulina et al., Diabetes 2000 February; 49(2): 263-71). Inhibition of GSK3 increases the activity of glycogen synthase thereby decreasing glucose levels by its conversion to glycogen. In animal models of diabetes, GSK3 inhibitors lowered plasma glucose levels up to 50% (Cline et al., Diabetes, 2002, 51: 2903-2910; Ring et al., Diabetes 2003, 52: 588-595). GSK3 inhibition may therefore be of therapeutic relevance in the treatment of Type I and Type II diabetes and diabetic neuropathy.

### Alopecia

GSK3 phosphorylates and degrades β-catenin. β-catenin is an effector of the pathway for keratonin synthesis. β-catenin stabilisation may be lead to increase hair development. Mice expressing a stabilised β-catenin by mutation of sites phosphorylated by GSK3 undergo a process resembling de novo hair morphogenesis (Gat et al., Cell, 1998, 95(5): 605-14)). The new follicles formed sebaceous glands and dermal papilla, normally established only in embryogenesis. Thus GSK3 inhibition may offer treatment for baldness.

### Inflammatory Disease

The discovery that GSK3 inhibitors provide anti-inflammatory effects has raised the possibility of using GSK3 inhibitors for therapeutic intervention in inflammatory diseases. (Martin et al., Nat. Immunol. 2005, 6(8): 777-784; Jope et al., Neurochem. Res. 2006, DOI 10.1007/s11064-006-9128-5)). Inflammation is a common feature of a broad range of conditions including Alzheimer's Disease and mood disorders.

### Cancer

GSK3 is overexpressed in ovarian, breast and prostate cancer cells and recent data suggests that GSK3b may have a role in contributing to cell proliferation and survival pathways in several solid tumor types. GSK3 plays an important role in several signal transduction systems which influence cell proliferation and survival such as WNT, PI3 kinase and NFkB. GSK3b deficient MEFs indicate a crucial role in cell survival mediated NFkB pathway (Ougolkov A V and Billadeau D D., Future Oncol. 2006 February; 2(1): 91-100). Thus, GSK3 inhibitors may inhibit growth and survival of solid tumors, including pancreatic, colon and prostate cancer.

### Bone-Related Disorders and Conditions

It has been shown that GSK3 inhibitors could be used for treatment of bone-related disorders. This has been discussed in e.g. Tobias et al., Expert Opinion on Therapeutic Targets, February 2002, pp 41-56. GSK3 inhibitors could be used for treatment of bone-related disorders or other conditions, which involves a need for new and increased bone formation. Remodeling of the skeleton is a continuous process, controlled by systemic hormones such as parathyroid hormone (PTH), local factors (e.g. prostaglandin E2), cytokines and other biologically active substances. Two cell types are of key importance: osteoblasts (responsible for bone formation) and osteoclasts (responsible for bone resorption). Via the RANK, RANK ligand and osteoprotegerin regulatory system these two cell types interact to maintain normal bone turnover (Bell N H, Current Drug Targets—Immune, Endocrine & Metabolic Disorders, 2001, 1:93-102).

Osteoporosis is a skeletal disorder in which low bone mass and deterioration of bone microarchitecture lead to increased bone fragility and fracture risk. To treat osteoporosis, the two main strategies are to either inhibit bone resorption or to stimulate bone formation. The majority of drugs currently on the market for the treatment of osteoporosis act to increase bone mass by inhibiting osteoclastic bone resorption. It is recognized that a drug with the capacity to increase bone formation would be of great value in the treatment of osteoporosis as well as having the potential to enhance fracture healing in patients.

Recent in vitro studies suggest a role of GSK3β in osteoblast differentiation. First, it has been shown that glucocorticoids inhibit cell cycle progression during osteoblast differentiation in culture. The mechanism behind this is activation of GSK3β in osteoblasts, resulting in c-Myc down-regulation and impediment of the G₁/S cell cycle transition. The attenuated cell cycle and reduced c-Myc level are returned to normal when GSK3β is inhibited using lithium chloride (Smith et al., J. Biol. Chem., 2002, 277: 18191-18197). Secondly, inhibition of GSK3β in the pluripotent mesenchymal cell line C3H10T1/2 leads to a significant increase in endogenous β-catenin signaling activity. This, in turn, induces expression of alkaline phosphatase mRNA and protein, a marker of early osteoblast differentiation (Bain et al., Biochem. Biophys. Res. Commun., 2003, 301: 84-91).

### DISCLOSURE OF THE INVENTION

The present invention relates to a compound of formula (I):

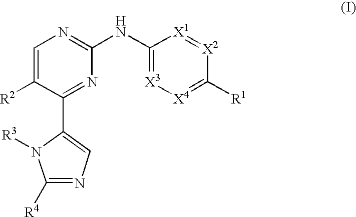

wherein:
R¹ is selected from sulphamoyl, carbamoyl, a group —R⁵-R⁶ and a nitrogen linked 4-7 membered saturated ring which optionally contains an additional nitrogen, oxygen or sulphur atom; wherein said ring is optionally substituted on carbon by one or more R⁷; and wherein if said ring contains an additional nitrogen atom that nitrogen is optionally substituted by R⁸;
at least one of X¹, X², X³ and X⁴ is selected from N, the other three X¹, X², X³ or X⁴ are independently selected from N or C(R⁹), provided that not more than two of X¹, X², X³ or X⁴ are selected from N;
R² is halo or cyano;
R³ is methyl, 3-tetrahydropyranyl or 4-tetrahydropyranyl, wherein the tetrahydropyranyl group is optionally substituted on carbon by one or more R¹⁰;
R⁴ is selected from hydrogen, halo, cyano and C₁₋₃alkyl, wherein C₁₋₃alkyl is optionally substituted with one or more halo;
R⁵ is selected from —O—, —C(O)—, —C(O)O—, —C(O)N(R¹¹)—, —S(O)ᵣ— and —SO₂N(R¹²)—; wherein R¹¹ and R¹² are independently selected from hydrogen or C₁₋₆alkyl and said alkyl is optionally substituted by one or more R¹³; and r is 0, 1 or 2;
R⁶ is selected from C₁₋₆alkyl, carbocyclyl and heterocyclyl; wherein R⁶ is optionally substituted on carbon by one or more R⁴; and wherein if said heterocyclyl contains an —NH— moiety that nitrogen is optionally substituted by a group selected from R¹⁵;
R⁷ is selected from halo, cyano, hydroxy, trifluoromethoxy, C₁₋₃alkoxy and C₁₋₃alkyl, wherein said C₁₋₃alkyl is optionally substituted by one or more halo;
R⁹ is selected from hydrogen, halo, cyano, hydroxy, amino, C₁₋₃alkyl and C₁₋₃alkoxy;
R¹⁰, R¹³ and R¹⁴ are independently selected from halo, cyano, hydroxy, amino, sulphamoyl, is C₁₋₆alkyl, C₁₋₆alkoxy, C₁₋₆alkoxyC₁₋₆alkoxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, N—(C₁₋₆alkyl)sulphamoyl, N,N—(C₁₋₆alkyl)₂sulphamoyl, C₁₋₆alkylsulphonylamino, carbocyclyl, heterocyclyl, carbocyclylC₁₋₃alkyl-R¹⁶—, heterocyclylC₁₋₃alkyl-R¹⁷—, carbocyclyl-R¹⁸— and heterocyclyl-R¹⁹—; wherein R¹⁰, R¹³ and R¹⁴ are independently of each other substituted on carbon by one or more R²⁰; and wherein if said heterocyclyl contains an —NH— moiety that nitrogen is optionally substituted by a group selected from R²¹;
R¹⁶, R¹⁷, R¹⁸ and R¹⁹ are independently selected from —O—, —N(R²²)—, —C(O)—, —N(R²³)C(O)—, —C(O)N(R²⁴)—, —S(O)ₛ—, —SO₂N(R²⁵)— and —N(R²⁶)SO₂—; wherein R²², R²³, R²⁴, R²⁵ and R²⁶ are independently selected from hydrogen and C₁₋₆alkyl; and s is 0, 1 or 2;
R⁸, R¹⁵ and R²¹ are independently selected from C₁₋₄alkyl, carbocyclyl, heterocyclyl, —C₁₋₄alkylcarbocyclyl, —C₁₋₄alkylheterocyclyl, C₁₋₄alkanoyl, C₁₋₄alkylsulphonyl and C₁₋₄alkoxycarbonyl; wherein R⁸, R¹⁵ and R²¹ independently of each other may be optionally substituted on carbon by one or more R²⁷; and
R²⁰ and R²⁷ are independently selected from halo, cyano, hydroxy, trifluoromethoxy, trifluoromethyl, amino, methyl, ethyl, phenyl, cyclopropyl, cyclobutyl, methoxy, ethoxy, methylamino, ethylamino, dimethylamino, diethylamino, mesyl, ethylsulphonyl and phenyl;
as a free base or a pharmaceutically acceptable salt thereof.

One aspect of the present invention relates to a compound of formula (I), wherein R¹ is a group —R⁵-R⁶ or a nitrogen linked 4-7 membered saturated ring which optionally contains an additional nitrogen, oxygen or sulphur atom; wherein said ring may be optionally substituted on carbon by one or more R⁷; and wherein if said ring contains an additional nitrogen atom that nitrogen is optionally substituted by R⁸;

at least one of X¹, X², X³ and X⁴ is selected from N, the other three X¹, X², X³ or X⁴ are independently selected from N or C(R⁹) provided that not more than two of X¹, X², X³ or X⁴ are selected from N;
R² is halo or cyano;
R³ is methyl or 4-tetrahydropyranyl, wherein said tetrahydropyranyl group is optionally substituted on carbon by one or more R¹⁰;
R⁴ is selected from hydrogen, halo, cyano and C₁₋₃alkyl, wherein said C₁₋₃alkyl is optionally substituted with one or more halo;
R⁵ is selected from —O—, —C(O)—, —C(O)O—, —C(O)N(R¹¹)—, —S(O)ᵣ— and —SO₂N(R¹²)—; wherein R¹¹ and R¹² are independently selected from hydrogen or C₁₋₆alkyl and said alkyl is optionally substituted by one or more R¹³; and r is 0 or 2;
R⁶ is selected from C₁₋₆alkyl, carbocyclyl and heterocyclyl; wherein R⁶ is optionally substituted on carbon by one or more R⁴; and wherein if said heterocyclyl contains an —NH— moiety that nitrogen is optionally substituted by a group selected from R¹⁵;
R⁷ is selected from halo, cyano, hydroxy, trifluoromethoxy, C₁₋₃alkoxy and C₁₋₃alkyl, wherein said C₁₋₃alkyl is optionally substituted by one or more halo;
R⁹ is selected from hydrogen, halo, cyano, hydroxy, C₁₋₃alkyl and C₁₋₃alkoxy;
R¹⁰, R¹³ and R¹⁴ are independently selected from halo, cyano, hydroxy, amino, sulphamoyl, C₁₋₆alkyl, C₁₋₆alkoxy, C₁₋₆alkoxyC₁₋₆alkoxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, N—(C₁₋₆alkyl)sulphamoyl, N,N—C₁₋₆alkyl)₂sulphamoyl, C₁₋₆alkylsulphonylamino, carbocyclyl, heterocyclyl, carbocyclylC₁₋₃alkyl-R¹⁶ heterocyclylC₁₋₃alkyl-R¹⁷—, carbocyclyl-R¹⁸— and heterocyclyl-R¹⁹—; wherein R¹⁰, R¹³ and R¹⁴ independently of each other are optionally substituted on carbon by one or more R²⁰; and wherein if said heterocyclyl contains an —NH— moiety that nitrogen is optionally substituted by a group selected from R²¹;
R¹⁶, R¹⁷, R¹⁸ and R¹⁹ are independently selected from —O—, —N(R²²)—, —C(O)—, —N(R²³)C(O)—, —C(O)N(R²⁴)—, —S(O)ₛ—, —SO₂N(R²⁵)— and —N(R²⁶)SO₂—; wherein R²², R²³, R²⁴, R²⁵ and R²⁶ are independently selected from hydrogen or C₁₋₆alkyl; and s is 0, 1 or 2;
R⁸, R¹⁵ and R²¹ are independently selected from C₁₋₄alkyl, carbocyclyl, heterocyclyl, —C₁₋₄alkylcarbocyclyl, —C₁₋₄alkylheterocyclyl, C₁₋₄alkanoyl, C₁₋₄alkylsulphonyl and C₁₋₄alkoxycarbonyl; wherein R⁸, R¹⁵ and R²¹ independently of each other may be optionally substituted on carbon by one or more R²⁷; and
R²⁰ and R²⁷ are independently selected from halo, cyano, hydroxy, trifluoromethoxy, trifluoromethyl, amino, methyl, ethyl, phenyl, cyclopropyl, cyclobutyl, methoxy, ethoxy, methylamino, ethylamino, dimethylamino, diethylamino, mesyl and ethylsulphonyl; as a free base or a pharmaceutically acceptable salt, an in vivo hydrolysable ester, solvate or solvate of a salt thereof.

Any or all of the compounds of the present invention have a potent inhibiting effect at GSK3 in addition to a selective inhibiting effect at GSK3.

Another aspect of the present invention relates to a compound of formula (I), wherein R² is halo.

Yet another aspect of the present invention relates to a compound of formula (I), wherein R² is fluoro.

Another aspect of the present invention relates to a compound of formula (I), wherein R³ is 4-tetrahydropyranyl or methyl.

Yet another aspect of the present invention relates to a compound of formula (I), wherein R⁴ is hydrogen or C₁₋₃alkyl, wherein said C₁₋₃alkyl is optionally substituted with one or more halo. According to one embodiment of the present invention, R⁴ is C₁₋₃alkyl. According to another embodiment of the present invention, R⁴ is methyl. According to one embodiment of the present invention, R⁴ is trifluoromethyl.

One aspect of the present invention relates to a compound of formula (I), wherein R⁵ is —C(O)— or —S(O)ᵣ—; and r is 0 or 2. According to one embodiment of the present invention, R⁵ is —C(O)—. According to one embodiment of the present invention, —S(O)ᵣ—; and r is 2.

One aspect of the present invention relates to a compound of formula (I), wherein R⁵ is —O— or C(O)O—.

Another aspect of the present invention relates to a compound of formula (I), wherein R⁵ is —C(O)N(R¹¹)— or —SO₂N(R¹²)—; wherein R¹¹ and R¹² are independently selected from hydrogen or C₁₋₆alkyl.

Yet another aspect of the present invention relates to a compound of formula (I), wherein R⁶ is C₁₋₆alkyl or heterocyclyl; wherein R⁶ is optionally substituted on carbon by one or more R¹⁴; and wherein if said heterocyclyl contains an —NH— moiety that nitrogen is optionally substituted by a group selected from R¹⁵. According to one embodiment of the present invention, said C₁₋₆alkyl is methyl, ethyl, butan-2-yl, butan-3-yl, propan-2-yl and tert-butyl. According to another embodiment of the present invention, said heterocyclyl is selected from morpholinyl, homomorpholinyl, piperidinyl, pyrrolidinyl, azetidinyl, piperazinyl, homopiperidinyl and homopiperazinyl. According to yet another embodiment of the present invention, said heterocyclyl is selected from piperidinyl, pyrrolidinyl, azetidinyl and piperazinyl

According to one embodiment of the present invention, R¹⁴ is C₁₋₆alkoxy, halo, C₁₋₆alkyl, carbocyclyl, heterocyclyl and N,N—(C₁₋₆alkyl)₂amino; wherein R¹⁴ is optionally substituted on carbon by one or more R²⁰.

According to one embodiment of the present invention, R¹⁵ is C₁₋₄alkyl or carbocycle; wherein R¹⁵ is optionally substituted on carbon by one or more R²⁷.

One aspect of the present invention relates to a compound of formula (I), wherein R⁸ is C₁₋₄alkyl, and wherein R⁸ may be optionally substituted on carbon by one or more R²⁷. According to one embodiment of the present invention, R²⁷ is hydroxy, halo, ethoxy, methoxy or phenyl.

Another aspect of the present invention relates to a compound of formula (I), wherein at least one of X², X³ and X⁴ is selected from N, the other two X², X³ or X⁴ are independently selected from N or C(R⁹). According to one embodiment of the present invention, X³ or X⁴ is N.

Yet another aspect of the present invention relates to a compound of formula (I), wherein R⁹ is hydrogen, methyl, trifluoromethyl, trifluoromethoxy or halo. According to one embodiment of the present invention, R⁹ is hydrogen. According to one embodiment of the present invention, wherein one of R⁹ is halo. According to another embodiment of the present invention, said halo is chloro.

Other suitable values of R¹⁰ are for example fluoro, cyano, methyl and ethyl and other suitable values of R¹¹ and R¹² are for example hydrogen and C₁₋₃alkyl.

One aspect of the present invention relates to a compound of formula (I), wherein R¹ is a group —R⁵-R⁶;

at least one of X¹, X², X³ and X⁴ is selected from N, the other three X¹, X², X³ or X⁴ are independently selected from N or C(R⁹), provided that not more than two of X¹, X², X³ or
X⁴ are selected from N;
R² is halo;
R³ is methyl or 4-tetrahydropyranyl;
R⁴ is C₁₋₃alkyl, wherein said C₁₋₃alkyl is optionally substituted with one or more halo;
R⁵ is selected from —O—, —C(O)—, —C(O)O—, —C(O)N(R¹¹)—, —S(O)ᵣ— and —SO₂N(R¹²)—; wherein R¹¹ and R¹² are independently selected from hydrogen or C₁₋₆alkyl and said alkyl is optionally substituted by one or more R¹³ and r is 2;
R⁶ is C₁₋₆alkyl or heterocyclyl; wherein R⁶ is optionally substituted on carbon by one or more R¹⁴; and wherein if said heterocyclyl contains an —NH— moiety that nitrogen is optionally substituted by a group selected from R¹⁵;
R⁹ is hydrogen or halo;
R¹⁴ is selected from halo, C₁₋₆alkyl, carbocycle, N,N—(C₁₋₆alkyl)₂amino, heterocyclyl and C₁₋₆alkoxy; wherein R¹⁴ is optionally on carbon by one or more R²⁰;
R¹⁵ is C₁₋₄alkyl or carbocycle; wherein R¹⁵ is optionally substituted on carbon by one or more R²⁷; and
R²⁰ and R²⁷ are independently selected from halo, methoxy, ethoxy, and phenyl.

Another aspect of the present invention relates to a compound according to claim 1 or 2, wherein, R¹ is a group —R⁵-R⁶; at least one of X¹, X², X³ and X⁴ is selected from N, the other three X¹, X², X³ or X⁴ are independently selected from N or C(R⁹), provided that not more than two of X¹, X², X³ or X⁴ are selected from N; R² is halo; R³ is 4-tetrahydropyranyl;

R⁴ is C₁₋₃alkyl; R⁵ is —C(O) or —S(O)ᵣ— and —SO₂N(R¹²)—; and r is 2; R⁶ is C₁₋₆alkyl or heterocyclyl; wherein if said heterocyclyl contains an —NH— moiety that nitrogen is optionally substituted by a group selected from R¹⁵; R⁹ is hydrogen; R¹⁵ is C₁₋₄alkyl.

The present invention also provides a compound selected from:
5-Fluoro-N-[5-(methylsulfonyl)pyridin-2-yl]-4-[2-methyl-1-(tetrahydro-2H-pyran-4-yl)-1H-imidazol-5-yl]pyrimidin-2-amine hydrochloride;
Azetidin-1-yl-[3-chloro-5-[[5-fluoro-4-[3-methyl-2-(trifluoromethyl)imidazol-4-yl]pyrimidin-2-yl]amino]pyridin-2-yl]methanone hydrochloride;
N-[5-Chloro-6-(piperidin-1-ylcarbonyl)pyridin-3-yl]-5-fluoro-4-[2-methyl-1-(tetrahydro-2H-pyran-4-yl)-1H-imidazol-5-yl]pyrimidin-2-amine hydrochloride;
N-[5-Chloro-6-(piperidin-1-ylcarbonyl)pyridin-3-yl]-4-(1,2-dimethyl-1H-imidazol-5-yl)-5-fluoropyrimidin-2-amine hydrochloride;
N-[5-Chloro-6-(piperidin-1-ylcarbonyl)pyridin-3-yl]-5-fluoro-4-[1-methyl-2-(trifluoromethyl)-1H-imidazol-5-yl]pyrimidin-2-amine hydrochloride; and
N-[5-Chloro-6-(piperidin-1-ylcarbonyl)pyridin-3-yl]-5-fluoro-4-[1-(tetrahydro-2H-pyran-4-yl)-2-(trifluoromethyl)-1H-imidazol-5-yl]pyrimidin-2-amine hydrochloride; or other pharmaceutically acceptable salts or free bases thereof.

The present invention also provides a compound selected from:
5-Fluoro-N-[6-(methylsulfonyl)pyridin-3-yl]-4-[2-methyl-1-(tetrahydro-2H-pyran-4-yl)-1H-imidazol-5-yl]pyrimidin-2-amine;
5-Fluoro-N-{5-[(4-methylpiperazin-1-yl)carbonyl]pyridin-2-yl}-4-[2-methyl-1-(tetrahydro-2H-pyran-4-yl)-1H-imidazol-5-yl]pyrimidin-2-amine;
5-Fluoro-N-{6-[(4-methylpiperazin-1-yl)carbonyl]pyridin-3-yl}-4-[2-methyl-1-(tetrahydro-2H-pyran-4-yl)-1H-imidazol-5-yl]pyrimidin-2-amine;
N-[6-(Azetidin-1-ylcarbonyl)pyridin-3-yl]-5-fluoro-4-[2-methyl-1-(tetrahydro-2H-pyran-4-yl)-1H-imidazol-5-yl]pyrimidin-2-amine;
(6-Ethoxy-pyridin-3-yl)-{5-fluoro-4-[2-methyl-3-(tetrahydro-pyran-4-yl)-3H-imidazol-4-yl]-pyrimidin-2-yl}-amine;
{5-Fluoro-4-[2-methyl-3-(tetrahydro-pyran-4-yl)-3H-imidazol-4-yl]-pyrimidin-2-yl}-(2-methoxy-pyrimidin-5-yl)-amine;
N-Butan-2-yl-5-[[5-fluoro-4-[2-methyl-3-(oxan-4-yl)imidazol-4-yl]pyrimidin-2-yl]amino]-N-propyl-pyridine-2-carboxamide;
(3,3-Difluoropyrrolidin-1-yl)-[5-[[5-fluoro-4-[2-methyl-3-(oxan-4-yl)imidazol-4-yl]pyrimidin-2-yl]amino]pyridin-2-yl]methanone;
[5-[[5-Fluoro-4-[2-methyl-3-(oxan-4-yl)imidazol-4-yl]pyrimidin-2-yl]amino]pyridin-2-yl]-(3-methyl-1-piperidyl)methanone;
5-[[5-Fluoro-4-[2-methyl-3-(oxan-4-yl)imidazol-4-yl]pyrimidin-2-yl]amino]-N-methyl-N-propan-2-yl-pyridine-2-carboxamide;
[5-[[5-Fluoro-4-[2-methyl-3-(oxan-4-yl)imidazol-4-yl]pyrimidin-2-yl]amino]pyridin-2-yl]-[4-(4-fluorophenyl)-1-piperidyl]methanone;
(4-Ethylpiperazin-1-yl)-[5-[[5-fluoro-4-[2-methyl-3-(oxan-4-yl)imidazol-4-yl]pyrimidin-2-yl]amino]pyridin-2-yl]methanone;
(4-Butylpiperazin-1-yl)-[5-[[5-fluoro-4-[2-methyl-3-(oxan-4-yl)imidazol-4-yl]pyrimidin-2-yl]amino]pyridin-2-yl]methanone;
N-Ethyl-5-[[5-fluoro-4-[2-methyl-3-(oxan-4-yl)imidazol-4-yl]pyrimidin-2-yl]amino]-N-propan-2-yl-pyridine-2-carboxamide;
[5-[[5-Fluoro-4-[2-methyl-3-(oxan-4-yl)imidazol-4-yl]pyrimidin-2-yl]amino]pyridin-2-yl]-(1-piperidyl)methanone;
[5-[[5-Fluoro-4-[2-methyl-3-(oxan-4-yl)imidazol-4-yl]pyrimidin-2-yl]amino]pyridin-2-yl]-(4-propan-2-ylpiperazin-1-yl)methanone;
5-[[5-Fluoro-4-[2-methyl-3-(oxan-4-yl)imidazol-4-yl]pyrimidin-2-yl]amino]-N,N-dipropan-2-yl-pyridine-2-carboxamide;
(2,6-Dimethyl-1-piperidyl)-[5-[[5-fluoro-4-[2-methyl-3-(oxan-4-yl)imidazol-4-yl]pyrimidin-2-yl]amino]pyridin-2-yl]methanone;
5-[[5-Fluoro-4-[2-methyl-3-(oxan-4-yl)imidazol-4-yl]pyrimidin-2-yl]amino]-N,N-dipropyl-pyridine-2-carboxamide;
[5-[[5-Fluoro-4-[2-methyl-3-(oxan-4-yl)imidazol-4-yl]pyrimidin-2-yl]amino]pyridin-2-yl]-(4-methoxy-1-piperidyl)methanone;
N-Ethyl-5-[[5-fluoro-4-[2-methyl-3-(oxan-4-yl)imidazol-4-yl]pyrimidin-2-yl]amino]-N-methyl-pyridine-2-carboxamide;
[5-[[5-Fluoro-4-[2-methyl-3-(oxan-4-yl)imidazol-4-yl]pyrimidin-2-yl]amino]pyridin-2-yl]-(4-methyl-1-piperidyl)methanone;
(4-Benzylpiperazin-1-yl)-[5-[[5-fluoro-4-[2-methyl-3-(oxan-4-yl)imidazol-4-yl]pyrimidin-2-yl]amino]pyridin-2-yl]methanone;
(4,4-Difluoro-1-piperidyl)-[5-[[5-fluoro-4-[2-methyl-3-(oxan-4-yl)imidazol-4-yl]pyrimidin-2-yl]amino]pyridin-2-yl]methanone;
N-Benzyl-5-[[5-fluoro-4-[2-methyl-3-(oxan-4-yl)imidazol-4-yl]pyrimidin-2-yl]amino]-N-propan-2-yl-pyridine-2-carboxamide;
5-[[5-Fluoro-4-[2-methyl-3-(oxan-4-yl)imidazol-4-yl]pyrimidin-2-yl]amino]-N-methyl-N-(2-methylpropyl)pyridine-2-carboxamide;
[5-[[5-Fluoro-4-[2-methyl-3-(oxan-4-yl)imidazol-4-yl]pyrimidin-2-yl]amino]pyridin-2-yl]-(4-fluoro-1-piperidyl)methanone;
N-Benzyl-N-ethyl-5-[[5-fluoro-4-[2-methyl-3-(oxan-4-yl)imidazol-4-yl]pyrimidin-2-yl]amino]pyridine-2-carboxamide;
(4-Butan-2-ylpiperazin-1-yl)-[5-[[5-fluoro-4-[2-methyl-3-(oxan-4-yl)imidazol-4-yl]pyrimidin-2-yl]amino]pyridin-2-yl]methanone;
N-(Cyclopropylmethyl)-5-[[5-fluoro-4-[2-methyl-3-(oxan-4-yl)imidazol-4-yl]pyrimidin-2-yl]amino]-N-propyl-pyridine-2-carboxamide;
[5-[[5-Fluoro-4-[2-methyl-3-(oxan-4-yl)imidazol-4-yl]pyrimidin-2-yl]amino]pyridin-2-yl]-[4-(4-fluorophenyl)piperazin-1-yl]methanone;
[5-[[5-Fluoro-4-[2-methyl-3-(oxan-4-yl)imidazol-4-yl]pyrimidin-2-yl]amino]pyridin-2-yl]-(4-propylpiperazin-1-yl)methanone;
N,N-Diethyl-5-[[5-fluoro-4-[2-methyl-3-(oxan-4-yl)imidazol-4-yl]pyrimidin-2-yl]amino]pyridine-2-carboxamide;
N-(3-Dimethylamino-2,2-dimethyl-propyl)-5-[[5-fluoro-4-[2-methyl-3-(oxan-4-yl)imidazol-4-yl]pyrimidin-2-yl]amino]pyridine-2-carboxamide;
(3,5-Dimethyl-1-piperidyl)-[5-[[5-fluoro-4-[2-methyl-3-(oxan-4-yl)imidazol-4-yl]pyrimidin-2-yl]amino]pyridin-2-yl]methanone;
Methyl 5-[[5-fluoro-4-[2-methyl-3-(oxan-4-yl)imidazol-4-yl]pyrimidin-2-yl]amino]pyridine-2-carboxylate;
Azetidin-1-yl-[3-chloro-5-[[5-fluoro-4-[2-methyl-3-(oxan-4-yl)imidazol-4-yl]pyrimidin-2-yl]amino]pyridin-2-yl]methanone;
[3-Chloro-5-[[5-fluoro-4-[3-(oxan-4-yl)-2-(trifluoromethyl)imidazol-4-yl]pyrimidin-2-yl]amino]pyridin-2-yl]-(4-methylpiperazin-1-yl)methanone;
[3-Chloro-5-[[5-fluoro-4-[3-methyl-2-(trifluoromethyl)imidazol-4-yl]pyrimidin-2-yl]amino]pyridin-2-yl]-(4-methylpiperazin-1-yl)methanone;
N-[6-(Azetidin-1-ylcarbonyl)pyridin-3-yl]-4-(1,2-dimethyl-1H-imidazol-5-yl)-5-fluoropyrimidin-2-amine;
4-(1,2-Dimethyl-1H-imidazol-5-yl)-5-fluoro-N-{6-[(4-methylpiperazin-1-yl)carbonyl]pyridin-3-yl}pyrimidin-2-amine;
N-[6-(Azetidin-1-ylcarbonyl)-5-chloropyridin-3-yl]-4-(1,2-dimethyl-1H-imidazol-5-yl)-5-fluoropyrimidin-2-amine;
N-{5-Chloro-6-[(4-methylpiperazin-1-yl)carbonyl]pyridin-3-yl}-4-(1,2-dimethyl-1H-imidazol-5-yl)-5-fluoropyrimidin-2-amine;
{5-Fluoro-4-[2-methyl-3-(tetrahydro-pyran-4-yl)-3H-imidazol-4-yl]-pyrimidin-2-yl}-[6-(propan-2-ylsulfonyl)-pyridin-3-yl]-amine;
(6-Ethanesulfonyl-pyridin-3-yl)-{5-fluoro-4-[2-methyl-3-(tetrahydro-pyran-4-yl)-3H-imidazol-4-yl]-pyrimidin-2-yl}-amine;
5-[[5-Fluoro-4-[2-methyl-3-(oxan-4-yl)-2,4-dihydroimidazol-4-yl]pyrimidin-2-yl]amino]-N-(2,2,2-trifluoroethyl)pyridine-2-sulfonamide;
N,N-Dimethyl-5-[[4-[2-methyl-3-(oxan-4-yl)-2,4-dihydroimidazol-4-yl]pyrimidin-2-yl]amino]pyridine-2-sulfonamide; and
{5-Fluoro-4-[2-methyl-3-(tetrahydro-pyran-4-yl)-3H-imidazol-4-yl]-pyrimidin-2-yl}-[6-(4-methyl-piperazine-1-sulfonyl)-pyridin-3-yl]-amine;
   as a free base or a pharmaceutically acceptable salt thereof.

The present invention also provides a compound selected from:
Lithium 5-[[5-fluoro-4-[2-methyl-3-(oxan-4-yl)imidazol-4-yl]pyrimidin-2-yl]amino]pyridine-2-carboxylate;
Azetidin-1-yl-(3,5-dichloropyridin-2-yl)methanone;
(3,5-Dichloropyridin-2-yl)-(4-methylpiperazin-1-yl)methanone;
5-Bromo-pyridine-2-sulfonic acid (2,2,2-trifluoro-ethyl)-amide;
1-(5-Bromo-pyridine-2-sulfonyl)-4-methyl-piperazine;
5-Bromo-pyridine-2-sulfonic acid dimethylamide; and
3,5-Dichloro-2-(piperidin-1-ylcarbonyl)pyridine.

Said compound(s) can be used as intermediates in processes for obtaining a compound of formula (I).

In this specification the term “alkyl” includes both straight and branched chain alkyl groups but references to individual alkyl groups such as “propyl” are specific for the straight chain version only. For example, “C₁₋₆alkyl” and “C₁₋₄alkyl” include methyl, ethyl, propyl, isopropyl and t-butyl. However, references to individual alkyl groups such as ‘propyl’ are specific for the straight-chained version only and references to individual branched chain alkyl groups such as ‘isopropyl’ are specific for the branched chain version only. A similar convention applies to other radicals, for example “carbocyclylC₁₋₃alkyl-R¹⁶” includes carbocyclylmethyl-R¹⁶, 1-carbocyclylethyl-R¹⁶ and 2-carbocyclylethyl-R¹⁶.

The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

A “4-7 membered saturated heterocyclic group” is a saturated monocyclic ring containing 4-7 atoms of which at least one atom is chosen from nitrogen, sulphur or oxygen, which may, unless otherwise specified, be carbon or nitrogen linked, wherein a —CH₂— group can optionally be replaced by a —C(O)— and a sulphur atom may be optionally oxidised to form the S-oxides. Examples and suitable values of the term “4-7 membered saturated heterocyclic group” are morpholino, piperidyl, 1,4-dioxanyl, 1,3-dioxolanyl, 1,2-oxathiolanyl, imidazolidinyl, pyrazolidinyl, piperazinyl, thiazolidinyl, pyrrolidinyl, thiomorpholino, homopiperazinyl and tetrahydropyranyl.

A “nitrogen linked 4-7 membered saturated ring which optionally contains an additional nitrogen, oxygen or sulphur atom” is a saturated monocyclic ring containing 4-7 atoms linked to the X¹-X⁴ containing ring of formula (I) via a nitrogen atom contained in the ring. The ring optionally contains an additional heteroatom selected from nitrogen, sulphur or oxygen, wherein a —CH₂— group can optionally be replaced by a —C(O)—, and the optional sulphur atom may be optionally oxidised to form the S-oxides. Particular examples of a “nitrogen linked 4-7 membered saturated ring which optionally contains an additional nitrogen, oxygen or sulphur atom” are piperazin-1-yl and morpholino, particularly morpholino.

A “heterocyclyl” is a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂— group can optionally be replaced by a —C(O)—, a ring nitrogen atom may optionally bear a C₁₋₆alkyl group and form a quaternary compound or a ring nitrogen and/or sulphur atom may be optionally oxidised to form the N-oxide and or the S-oxides. Examples and suitable values of the term “heterocyclyl” are morpholino, piperidyl, pyridyl, pyranyl, pyrrolyl, isothiazolyl, indolyl, quinolyl, thienyl, 1,3-benzodioxolyl, thiadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, pyrimidyl, pyrazinyl, pyridazinyl, isoxazolyl, N-methylpyrrolyl, 4-pyridone, 1-isoquinolone, 2-pyrrolidone, 4-thiazolidone, pyridine-N-oxide and quinoline-N-oxide. In one aspect of the invention a “heterocyclyl” is a saturated, partially saturated or unsaturated, mono or bicyclic ring containing 5 or 6 atoms of which at least one atom is chosen from nitrogen, sulphur or oxygen, it may, unless otherwise specified, be carbon or nitrogen linked, a —CH₂— group can optionally be replaced by a —C(O)— and a ring sulphur atom may be optionally oxidised to form the S-oxides.

A “carbocyclyl” is a saturated, partially saturated or unsaturated, mono or bicyclic carbon ring that contains 3-12 atoms; wherein a —CH₂— group can optionally be replaced by a —C(O)—. Particularly “carbocyclyl” is a monocyclic ring containing 5 or 6 atoms or a is bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl.

Examples of “C₁₋₆alkoxy” include methoxy, ethoxy and propoxy. Examples of “C₁₋₆alkanoylamino” include formamido, acetamido and propionylamino. Examples of “C₁₋₆alkylS(O)ₐ wherein a is 0, 1 or 2” include methylthio, ethylthio, methylsulphinyl, ethylsulphinyl, mesyl and ethylsulphonyl. Examples of “C₁₋₆alkanoyl” include propionyl and acetyl. Examples of “N—(C₁₋₆alkyl)amino” include methylamino and ethylamino. Examples of “N,N—(C₁₋₆alkyl)₂amino” include di-N-methylamino, di-(N-ethyl)amino and N-ethyl-N-methylamino. Examples of “N—(C₁₋₆alkyl)sulphamoyl” are N-(methyl)sulphamoyl and N-(ethyl)sulphamoyl. Examples of “N,N—(C₁₋₆alkyl)₂sulphamoyl” are N,N-(dimethyl)sulphamoyl and N-(methyl)-N-(ethyl)sulphamoyl. Examples of “N—(C₁₋₆alkyl)carbamoyl” are methylaminocarbonyl and ethylaminocarbonyl. Examples of “N,N—(C₁₋₆alkyl)₂carbamoyl” are dimethylaminocarbonyl and methylethylaminocarbonyl. Examples of “C₁₋₆alkylsulphonylamino” include methylsulphonylamino, isopropylsulphonylamino and t-butylsulphonylamino. Examples of “C₁₋₆alkylsulphonyl” include methylsulphonyl, isopropylsulphonyl and t-butylsulphonyl.

The terms “—C₁₋₄alkylcarbocyclyl” and “—C₁₋₄alkylheterocyclyl” includes both straight and branched chain alkyl groups of between one and four carbon atoms that then link to a carbocycle or heterocycle respectively. The terms carbocycle and heterocycle are as defined above. Non-limiting examples of C₁₋₄alkylcarbocyclyl therefore include benzyl, 2-phenylethyl, 1-phenylethyl, cyclopropylmethyl and cyclohexylethyl. Non-limiting examples of C₁₋₄alkylheterocyclyl include pyridin-3-ylmethyl, oxolan-2yl-methyl, 2-(4-piperidyl)ethyl and 1-thiophen-2-ylethyl.

A suitable pharmaceutically acceptable salt of a compound of the present invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Some compounds of the formula (I) may have stereogenic centres and/or geometric isomeric centres (E- and Z-isomers), and it is to be understood that the invention encompasses all such optical, diastereoisomers and geometric isomers that possess GSK3 inhibitory activity.

The invention relates to any and all tautomeric forms of the compounds of the formula (I) that possess GSK3 inhibitory activity.

The definition of compounds of formula (I) also includes in vivo hydrolysable esters, solvates or solvates of salts thereof.

It is also to be understood that certain compounds of the formula (I) can exist in solvated as well as unsolvated forms such as, for example, hydrated forms. It is to be understood that the invention encompasses all such solvated forms that possess GSK3 inhibitory activity.

### Methods of Preparation

The present invention also provides a process for preparing a compound of formula (I), or a pharmaceutically acceptable salt thereof or in vivo hydrolysable ester thereof, which process comprises:

a) reacting a pyrimidine of formula (II):

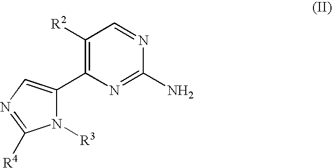

with a compound of formula (III):

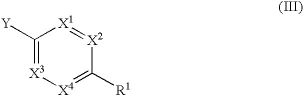

wherein Y is a displaceable group; and
R¹, R², R³, X¹, X², X³ and X⁴ are, unless otherwise specified, as defined in formula (I);
and thereafter optionally:
b) converting a compound of the formula (I) into another compound of formula (I);
c) removing any protecting groups; and
d) forming a pharmaceutically acceptable salt or in vivo hydrolysable ester.

Y is, as mentioned above, a displaceable group. Suitable values for Y are, for example, a halo (such as a chloro, bromo or iodo) or sulphonyloxy group (such as trifluoromethanesulphonyloxy group). According to one embodiment of the present invention, Y is chloro, bromo or iodo.

Specific reaction conditions for the above reactions are as follows:

Step a): Amines of formula (II) and compounds of formula (III) may be reacted together under standard Buchwald-Hartwig conditions, (for example see J. Am. Chem. Soc., 118, 7215; J. Am. Chem. Soc., 119, 8451; J. Am. Chem. Soc., 125, 6653; J. Org. Chem., 62, 1568 and 6066) for example in the presence of palladium acetate, in a suitable solvent for example an aromatic solvent such as toluene, benzene or xylene, with a suitable base for example an inorganic base such as caesium carbonate or an organic base such as potassium-t-butoxide, in the presence of a suitable ligand such as 2,2′-bis(diphenylphosphino)-1,1′-binaphthyl or 2-dicyclohexylphosphino-2′,4′,6′-triiso-propyl-1,1′-biphenyl and at a temperature in the range of 25 to 80° C.

Pyrimidines of the formula (II), wherein R³ is methyl; and R² and R⁴ are as defined in formula (I), may be prepared according to Scheme 1:

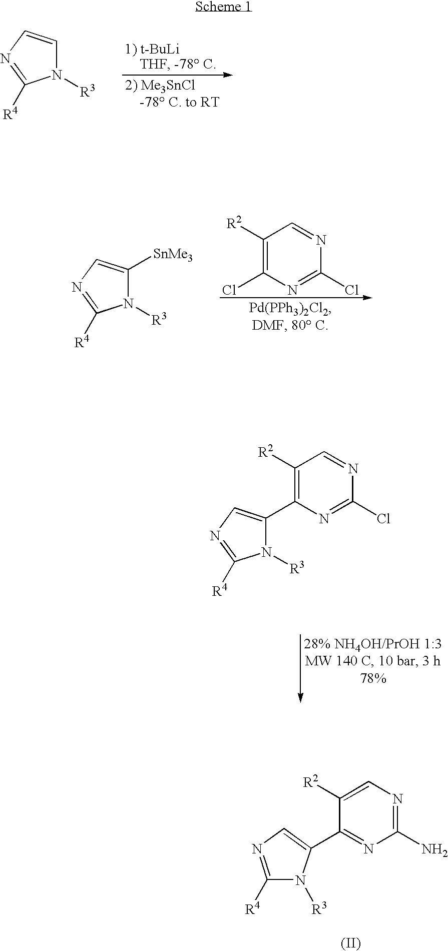

A synthesis of pyrimidines of formula (II) is described in Scheme 2, wherein R^{x} is selected from the same or different C₁₋₆alkyl and R², R³ and R⁴ are as defined in formula (I).

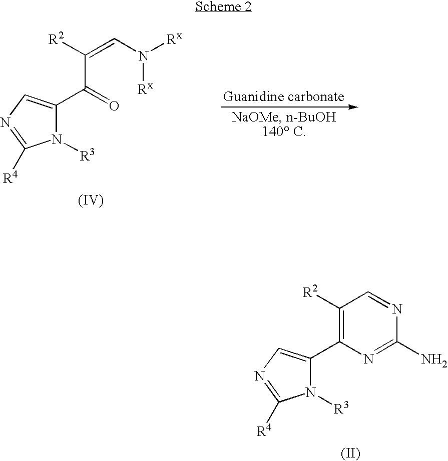

Compounds of formula (III) are commercially available compounds, or they are known in the literature, or they can be prepared by standard processes known in the art.

Compounds of formula (IV) in which R³ has the general structure R^{a}—CH—R^{b}, wherein R^{a} and R^{b} are hydrogen or form together a tetrahydropyran ring, wherein R⁴ is hydrogen or C₁₋₃alkyl, wherein said C₁₋₃alkyl may optionally be substituted with one or more halo and wherein R² is fluoro and R^{x} is as defined above, may be prepared according to Scheme 3, wherein,

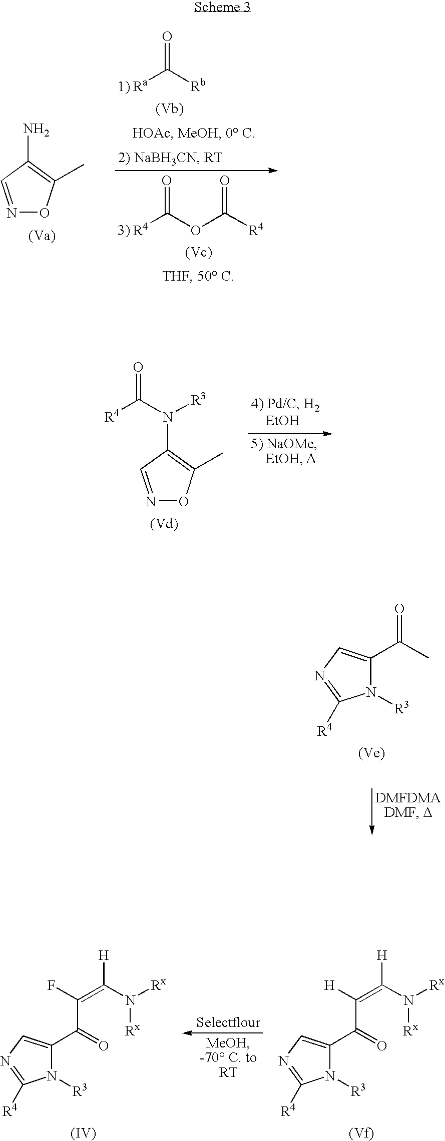

Compounds of formula (Va), (Vb) and (Vc) are commercially available compounds, or they are known in the literature, or they can be prepared by standard processes known in the art. Compound (Vf) can exist in either E or Z conformation around the alkene.

A compound of formula (Ia) can be prepared by reacting an acid intermediate (VI) with primary or secondary amines as shown in Scheme 4. This reaction can be achieved by mixing the acid or carboxylate salt with a coupling agent in a polar, aprotic solvent followed by addition of the primary or secondary amine. The amidation conditions involve, for example, taking a mixture of the carboxylate or acid, a coupling agent (such as HBTU or CDI), a base, such as DIPEA, together in a solvent such as DCM, N-methyl pyrrolidinone or dimethylformamide and then adding the amine at room temperature. In this example C(O)NR²⁸R²⁹ is defined as —R⁵-R⁶ above.

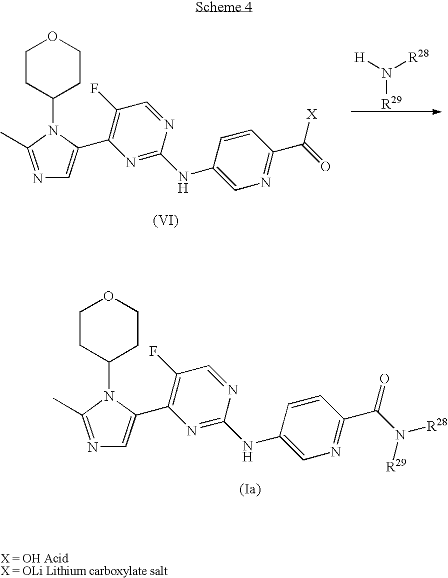

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halo group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Greene, Protective Groups in Organic Synthesis, John Wiley and Sons, 1999). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group that may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well-known in the chemical art.

### General Methods

All solvents used were analytical grade and commercially available anhydrous solvents were routinely used for reactions. Reactions were typically run under an inert atmosphere of nitrogen or argon.

¹H, ¹⁹F and ¹³C NMR spectra were recorded on a Varian Unity+400 NMR Spectrometer equipped with a 5 mm BBO probehead with Z-gradients, or a Varian Gemini 300 NMR spectrometer equipped with a 5 mm BBI probehead, or a Bruker Avance 400 NMR spectrometer equipped with a 60 μl dual inverse flow probehead with Z-gradients, or a Bruker DPX400 NMR spectrometer equipped with a 4-nucleus probehead equipped with Z-gradients, or a Bruker Avance 600 NMR spectrometer equipped with a 5 mm BBI probehead with Z-gradients. Unless specifically noted in the examples, spectra were recorded at 400 MHz for proton, 376 MHz for fluorine-19 and 100 MHz for carbon-13.

The following reference signals were used: the middle line of DMSO-d₆ δ 2.50 (1H), δ 39.51 (13C); the middle line of CD₃OD δ 3.31 (1H) or δ 49.15 (13C); CDCl₃ δ 7.26 (1H) and the middle line of CDCl₃ δ 77.16 (13C) (unless otherwise indicated). NMR spectra are reported either from high to low field or from low to high field.

Mass spectra were recorded on a Waters LCMS consisting of an Alliance 2795 (LC), Waters PDA 2996 and a ZQ single quadrupole mass spectrometer. The mass spectrometer was equipped with an electrospray ion source (ESI) operated in a positive or negative ion mode. The capillary voltage was 3 kV and cone voltage was 30 V. The mass spectrometer was scanned between m/z 100-700 with a scan time of 0.3 s. Separations were performed on either Waters X-Terra MS C8 (3.5 μm, 50 or 100 mm×2.1 mm i.d.) or an ACE 3 AQ (100 mm×2.1 mm i.d.) obtained from ScantecLab. Flow rates were regulated to 1.0 or 0.3 mL/min, respectively. The column temperature was set to 40° C. A linear gradient was applied using a neutral or acidic mobile phase system, starting at 100% A (A: 95:5 10 mM NH₄OAc:MeCN, or 95:5 8 mM HCOOH:MeCN) ending at 100% B (MeCN).

Alternatively, mass spectra were recorded on a Waters LCMS consisting of an Alliance 2690 Separations Module, Waters 2487 Dual 1 Absorbance Detector (220 and 254 nm) and a Waters ZQ single quadrupole mass spectrometer. The mass spectrometer was equipped with an electrospray ion source (ESI) operated in a positive or negative ion mode. The capillary voltage was 3 kV and cone voltage was 30 V. The mass spectrometer was scanned between m/z 97-800 with a scan time of 0.3 or 0.8 s. Separations were performed on a Chromolith Performance RP-18e (100×4.6 mm). A linear gradient was applied starting at 95% A (A: 0.1% HCOOH (aq.)) ending at 100% B (MeCN) in 5 minutes. Flow rate: 2.0 mL/min.

Microwave heating was performed in a single-mode microwave cavity producing continuous irradiation at 2450 MHz.

HPLC analyses were performed on an Agilent HP1000 system consisting of G1379A Micro Vacuum Degasser, G1312A Binary Pump, G1367A Well plate auto-sampler, G1316A Thermostatted Column Compartment and G1315B Diode Array Detector. Column: X-Terra MS, Waters, 3.0×100 mm, 3.5 μm. The column temperature was set to 40° C. and the flow rate to 1.0 ml/min. The Diode Array Detector was scanned from 210-300 nm, step and peak width were set to 2 nm and 0.05 min, respectively. A linear gradient was applied, starting at 100% A (A: 95:5 10 mM NH₄OAc:MeCN) and ending at 100% B (B: MeCN), in 4 min.

Alternatively, HPLC analyses were performed on a Gynkotek P580 HPG consisting of gradient pump with a Gynkotek UVD 170S UV-vis.-detector equipped with a Chromolith Performance RP column (C18, 100 mm×4.6 mm). The column temperature was set to +25° C. A linear gradient was applied using MeCN/0.1 trifluoroacetic acid in MilliQ water, run from 10% to 100% MeCN in 5 minutes. Flow rate: 3 ml/min.

A typical workup procedure after a reaction consisted of extraction of the product with a solvent such as ethyl acetate, washing with water followed by drying of the organic phase over MgSO₄ or Na₂SO₄, filtration and concentration of the solution in vacuo.

Thin layer chromatography (TLC) was performed on Merck TLC-plates (Silica gel 60 F₂₅₄) and UV visualized the spots. Flash chromatography was performed on a Combi Flash® Companion™ using RediSep™ normal-phase flash columns. Typical solvents used for flash chromatography were mixtures of chloroform/methanol, DCM/methanol, heptane/ethyl acetate, chloroform/methanol/ammonia (aq.) and DCM/methanol/NH₃ (aq.). SCX ion exchange columns were performed on Isolute® columns. Chromatography through ion exchange columns were typically performed in solvents such a methanol.

Preparative chromatography was run on a Waters autopurification HPLC with a diode array detector. Column: XTerra MS C8, 19×300 mm, 10 μm. Narrow gradients with MeCN/(95:5 0.1M NH₄OAc:MeCN) were used at a flow rate of 20 ml/min. Alternatively, purification was achieved on a semi preparative Shimadzu LC-8A HPLC with a Shimadzu SPD-10A UV-vis.-detector equipped with a Waters Symmetry® column (C18, 5 μm, 100 mm×19 mm). Narrow gradients with MeCN/0.1% trifluoroacetic acid in MilliQ Water were used at a flow rate of 10 ml/min.

The formation of hydrochloride salts of the final products were typically performed in solvents or solvents mixtures such as diethyl ether, tetrahydrofuran, DCM/toluene, DCM/methanol, followed by addition of 1M hydrogen chloride in diethyl ether.

The following abbreviations have been used:
aq. aqueous;
CDI carbonyl diimidazole;
CHCl₃ chloroform;
CDCl₃ deuterated chloroform;
CH₂Cl₂ dichloromethane;
Cs₂CO₃ caesium carbonate;
DCM dichloromethane;
DIPEA N,N-diisopropylethylamine;
DMF N,N-dimethylformamide;
DMFDMA dimethylformamide dimethylacetal;
DMSO dimethyl sulphoxide;
EtOAc ethyl acetate;
EtOH ethanol;
HBTU O-Benzotriazole-N,N,N′,N′-tetramethyl-uronium-hexafluoro-phosphate HOAc acetic acid;
HCOOH formic acid;
MeCN acetonitrile;
MeOH methanol;
Me₃SnCl trimethyltin chloride;
MgSO₄ magnesium sulphate;
Min minutes;
NaBH₃CN sodium cyanoborohydride;
NaHCO₃ sodium bicarbonate;
NaOMe sodium methoxide;
Na₂SO₄ sodium sulphate;
n-BuOH n-butanol;
NH₃ ammonia;
NH₄OAc ammonium acetate;
NH₄OH ammonium hydroxide;
o.n. overnight
Pd/C palladium on carbon;
Pd(PPh₃)₂Cl₂ bis(triphenylphosphine)palladium dichloride;
Pd₂(dba)₃ tris(dibenzylideneacetone)dipalladium;
PrOH propan-1-ol;
r.t. or RT room temperature;
Ret. T retention time;
Selectfluor N-fluoro-N′-chloromethyl-triethylenediamine-bis(tetrafluoroborate);
THF tetrahydrofuran;
t-BuLi tert-butyllithium;
Xantphos 9,9-Dimethyl-4,5-bis(diphenylphosphino)xanthene; and
X-Phos 2-dicyclohexylphosphino-2′,4′,6′-triiso-propyl-1,1′-biphenyl.

Starting materials used were either available from commercial sources or prepared according to literature procedures and had experimental data in accordance with those reported.

Compounds have been named either using ACD/Name, versions 8.08 or 9, software from Advanced Chemistry Development, Inc. (ACD/Labs), Toronto ON, Canada, www.acdlabs.com, 2004 or named according to the IUPAC convention.

### General Methods A to C

In the following general methods A to C, the groups R¹, R², R³, R⁴, X¹, X², X³, X⁴ and Y are used independently to indicate the diversity of substitution within each structure. The identity of R¹, R², R³, R⁴, X¹, X², X³, X⁴ and Y will be clear to a person skilled in the art based on the starting materials and intermediates for each specific example. For instance in Example 1, which refers to General method A, A1 is 5-fluoro-4-[2-methyl-1-(tetrahydro-2H-pyran-4-yl)-1H-imidazol-5-yl]pyrimidin-2-amine such that R³ is 4-tetrahydropyranyl and R⁴ is methyl and A2 is 2-bromo-5-(methylsulfonyl)pyridine such that X¹ is N, X², X³ and X⁴ are CH and R¹ is sulphonylmethane.

### General Method A

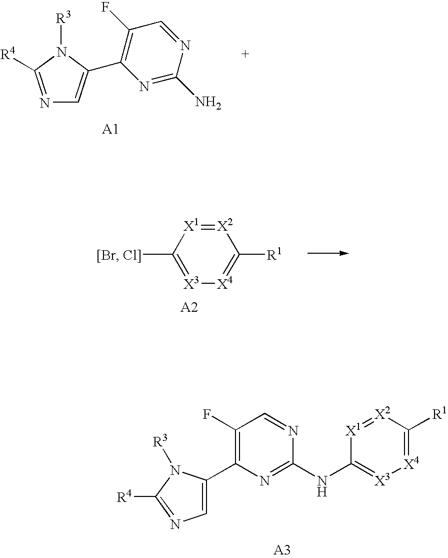

A1 (1.01-1.27 equiv.), A2 (1.0 equiv.) and Cs₂CO₃ (1.6-2.25 equiv.) were mixed in anhydrous 1,4-dioxane and the mixture was flushed with argon for 5 minutes before Pd₂(dba)₃ (0.05-0.2 equiv.) and X-Phos or Xantphos (0.10-0.20 equiv.) were added. The mixture was flushed with argon, then heated in a sealed tube at +90-+100° C. until the reaction was complete. Workup was done according to one of the following procedures: 1) The reaction mixture was diluted with a mixture of H₂O/CH₂Cl₂, the product was extracted with CH₂Cl₂, the combined organic phase was dried (Mg₂SO₄), filtered and concentrated. 2) The reaction mixture was diluted with CH₂Cl₂, filtered and concentrated. 3) The solvent was removed in vacuo and the residue was taken up in CH₂Cl₂ and washed with diluted NaHCO₃ (aq.) or water. The organic layer was dried (Na₂SO₄), filtered and concentrated. Purification was performed using preparative HPLC or chromatography on silica. Either the freebase or HCl salt was prepared.

### General Method B

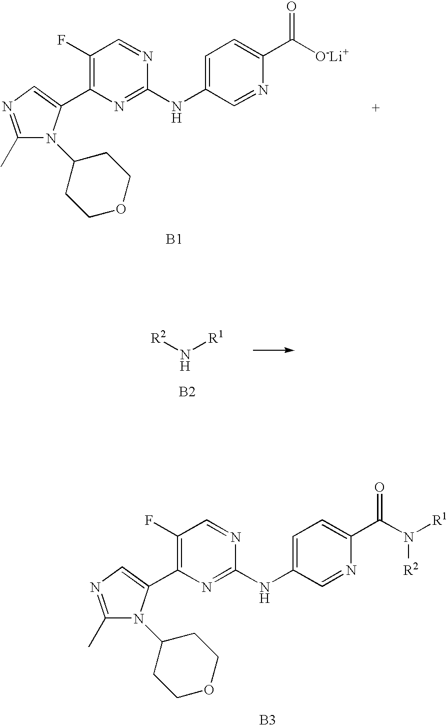

To a solution of B1 (0.12 mmol, 1.0 equiv.) in anhydrous DMF (0.65 mL) were added HBTU (59 mg, 0.15 mmol, 1.2 equiv.), amine B2 or a salt thereof (0.16 mmol, 1.3 equiv.) and DIPEA (48 mg, 0.37 mmol, 3 equiv. for free amines and 1 additional equiv. for each equiv. of salt). The reaction mixture was shaken o.n. at r.t. The crude product was purified by preparative HPLC.

### General Method C

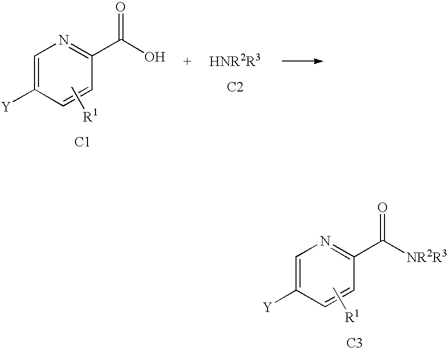

Thionyl chloride (5 mL) was added to C1 (1.0 equiv.). After addition of 2 drops of anhydrous DMF, the reaction mixture was refluxed for 30 minutes under an atmosphere of nitrogen. The solvent was evaporated in vacuo and the residue was dissolved in CH₂Cl₂ (until a clear solution was obtained). C2 (1.0 equiv.) was added dropwise followed by addition of triethylamine (1.0 equiv.). The reaction mixture was stirred at r.t. for 30 minutes before it was diluted with CH₂Cl₂, washed with saturated NaHCO₃ (aq.), dried (Na₂SO₄) and filtered. The solvent was evaporated in vacuo and the crude product was purified using flash column chromatography.

### EXAMPLES

The present invention will further be described in more detail by the following Examples, which are not to be construed as limiting the present invention.

### Example 1

### 5-Fluoro-N-[5-(methylsulfonyl)pyridin-2-yl]-4-[2-methyl-1-(tetrahydro-2H-pyran-4-yl)-1H-imidazol-5-yl]pyrimidin-2-amine hydrochloride

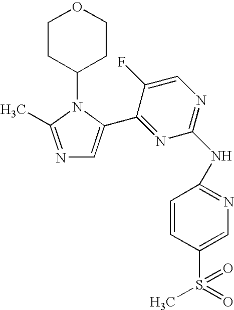

The title compound was prepared in accordance with general method A using 5-fluoro-4-[2-methyl-1-(tetrahydro-2H-pyran-4-yl)-1H-imidazol-5-yl]pyrimidin-2-amine (as described in Example 6) (50 mg, 0.18 mmol) and 2-bromo-5-(methylsulfonyl)pyridine (42 mg, 0.18 mmol) to give the title compound (34 mg, 44%).

¹H NMR (CDCl₃) δ ppm 9.19 (s, 1H) 8.91 (d, J=2.02 Hz, 1H) 8.48-8.53 (m, 2H) 8.12 (dd, J=8.84, 2.53 Hz, 1H) 7.65 (d, J=3.79 Hz, 1H) 5.10 (tt, J=12.28, 4.26 Hz, 1H) 4.10 (dd, J=11.62, 4.29 Hz, 2H) 3.34-3.44 (m, 2H) 3.09 (s, 3H) 2.66 (s, 3H) 2.46 (qd, J=12.46, 4.55 Hz, 2H) 1.91 (dd, J=12.25, 2.65 Hz, 2H); MS (ES) m/z 433 (M+1).

### Example 2

### 5-Fluoro-N-[6-(methylsulfonyl)pyridin-3-yl]-4-[2-methyl-1-(tetrahydro-2H-pyran-4-yl)-1H-imidazol-5-yl]pyrimidin-2-amine

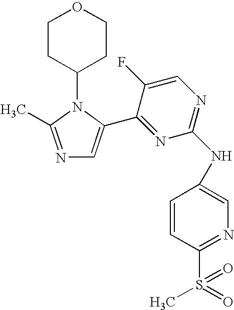

The title compound was prepared in accordance with general method A using 5-fluoro-4-[2-methyl-1-(tetrahydro-2H-pyran-4-yl)-1H-imidazol-5-yl]pyrimidin-2-amine (as described in Example 6) (50 mg, 0.18 mmol) and 5-bromo-2-(methylsulfonyl)pyridine (42 mg, 0.18 mmol) to give the title compound (36 mg, 46%). ¹H NMR (chloroform −d) δ ppm 8.85 (d, J=2.53 Hz, 1H) 8.34-8.39 (m, 2H) 8.11 (s, 1H) 8.00 (d, J=8.84 Hz, 1H) 7.69 (d, J=3.79 Hz, 1H) 4.99-5.09 (m, 1H) 4.10 (dd, J=11.62, 4.80 Hz, 2H) 3.36 (td, J=11.87, 1.77 Hz, 2H) 3.20 (s, 3H) 2.65 (s, 3H) 2.48-2.60 (m, 2H) 1.87 (dd, J=12.38, 3.28 Hz, 2H); MS (ES) m/z 433 (M+1).

### Example 3

### 5-Fluoro-N-{5-[(4-methylpiperazin-1-yl)carbonyl]pyridin-2-yl}-4-[2-methyl-1-(tetrahydro-2H-pyran-4-yl)-1H-imidazol-5-yl]pyrimidin-2-amine

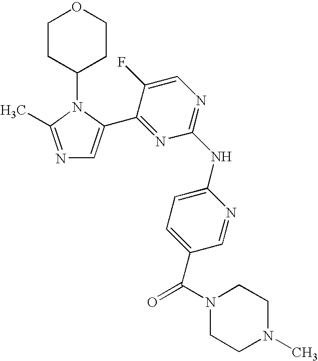

The title compound was prepared in accordance with general method A using 5-fluoro-4-[2-methyl-1-(tetrahydro-2H-pyran-4-yl)-1H-imidazol-5-yl]pyrimidin-2-amine (as described in Example 6) (35 mg, 0.13 mmol) and 1-[(6-chloropyridin-3-yl)carbonyl]-4-methylpiperazine (reported in WO 2003082853) (27 mg, 0.11 mmol) to give the title compound (60 mg, 100%). MS (ES, retention time: 2.53 min) m/z 385 (M+1).

### Example 4

### 5-Fluoro-N-{6-[(4-methylpiperazin-1-yl)carbonyl]pyridin-3-yl}-4-[2-methyl-1-(tetrahydro-2H-pyran-4-yl)-1H-imidazol-5-yl]pyrimidin-2-amine

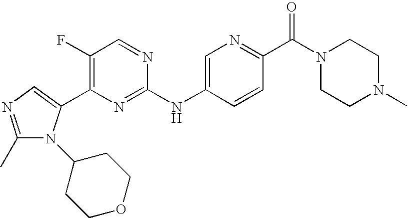

The title compound was prepared in accordance with general method A using 5-fluoro-4-[2-methyl-1-(tetrahydro-2H-pyran-4-yl)-1H-imidazol-5-yl]pyrimidin-2-amine (as described in Example 6) (26 mg, 0.095 mmol) and 1-[(5-bromopyridin-2-yl)carbonyl]-4-methylpiperazine (obtained from Example 4b) (27 mg, 0.095 mmol) to give the title compound in 61% (28 mg) yield.

¹H NMR (400 MHz, DMSO-d₆) δ ppm 9.95 (s, 1H) 8.79 (d, J=2.26 Hz, 1H) 8.64 (d, J=2.76 Hz, 1H) 8.11 (dd, J=8.66, 2.64 Hz, 1H) 7.55 (d, J=8.78 Hz, 1H) 7.35 (d, J=3.76 Hz, 1H) 5.03-4.91 (m, 1H) 3.81 (dd, J=11.42, 4.14 Hz, 2H) 3.67-3.56 (m, 2H) 3.56-3.47 (m, 2H) 3.11 (t, J=11.29 Hz, 2H) 2.54 (s, 3H) 2.40-2.31 (m, 2H) 2.31-2.24 (m, 2H) 2.18 (s, 3H) 2.24-2.10 (m, 2H) 1.78 (dd, J=12.17, 2.38 Hz, 2H). MS (ES) m/z 481 (M+1).

1-[(5-Bromopyridin-2-yl)carbonyl]-4-methylpiperazine was prepared as follows:

### Example 4(a)

### 5-Bromopyridine-2-carbonyl chloride

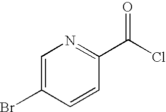

Thionylchloride (8.15 g, 68.5 mmol) and anhydrous DMF (catalytic amount) were added to 5-bromopyridine-2-carboxylic acid (0.50 g, 2.48 mmol) and the reaction mixture was refluxed until a clear solution was obtained. Excess thionylchloride was removed in vacuo to yield a crude product that was used directly without further purification or analysis.

### Example 4(b)

### 1-[(5-Bromopyridin-2-yl)carbonyl]-4-methylpiperazine

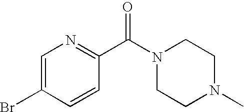

1-methylpiperazine (0.13 g, 1.3 mmol) and triethylamine (0.13 g, 1.3 mmol) were sequentially added to a stirred solution of 5-bromopyridine-2-carbonyl chloride (0.27 g, 1.24 mmol), obtained in example 4(a), in CH₂Cl₂ (5 mL) and the reaction was stirred at is ambient temperature until reaction was complete. The organic phase was diluted (CH₂Cl₂), washed with i) saturated aqueous NaHCO₃, ii) water. Absolute EtOH was then added followed by evaporation to dryness. The crude product was obtained in 89% (0.31 g) yield. This material was used in the next step (Example 4) without further purification.

¹H NMR (400 MHz, DMSO-d₆) δ ppm 8.72 (d, J=2.26 Hz, 1H) 8.18 (dd, J=8.41, 2.38 Hz, 1H) 7.55 (d, J=8.28 Hz, 1H) 3.68-3.58 (m, 2H) 3.40-3.33 (m, 2H) 2.40-2.33 (m, 2H) 2.29-2.22 (m, 2H) 2.19 (s, 3H). MS (ES) m/z 286 (⁸¹Br) (M+1).

### Example 5

### N-[6-(Azetidin-1-ylcarbonyl)pyridin-3-yl]-5-fluoro-4-[2-methyl-1-(tetrahydro-2H-pyran-4-yl)-1H-imidazol-5-yl]pyrimidin-2-amine

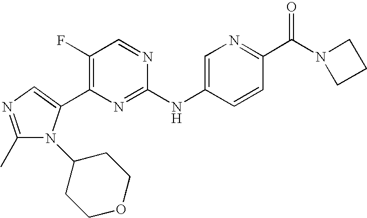

The title compound was prepared in accordance with general method A, with the exception that a second purification on a silica gel column was necessary to obtain a pure material, using 5-fluoro-4-[2-methyl-1-(tetrahydro-2H-pyran-4-yl)-1H-imidazol-5-yl]pyrimidin-2-amine (as described in Example 6) (36 mg, 0.13 mmol) and 2-(azetidin-1-ylcarbonyl)-5-bromopyridine (reported in WO 2005014571) (32 mg, 0.13 mmol) to give the title compound in 18% (10 mg) yield.

¹H NMR (400 MHz, DMSO-d₆) δ ppm 10.03 (s, 1H) 8.88 (d, J=2.26 Hz, 1H) 8.66 (d, J=2.51 Hz, 1H) 8.12 (dd, J=8.66, 2.64 Hz, 1H) 7.89 (d, J=8.53 Hz, 1H) 7.37 (d, J=3.51 Hz, 1H) 5.06-4.95 (m, 1H) 4.57 (t, J=7.65 Hz, 2H) 4.05 (t, J=7.70 Hz, 2H) 3.82 (dd, J=11.42, 4.14 Hz, 2H) 3.12 (t, J=11.04 Hz, 2H) 2.55 (s, 3H) 2.31-2.13 (m, 4H) 1.81 (dd, J=12.05, 2.26 Hz, 2H). MS (ES) m/z 438 (M+1).

The main intermediates were prepared as followed in Examples 6-9:

### Example 6

### 5-Fluoro-4-[2-methyl-1-(tetrahydro-2H-pyran-4-yl)-1H-imidazol-5-yl]pyrimidin-2-amine

### Example 6(a)

### 4-[N-Acetyl-N-(tetrahydro-2H-pyran-4-yl)]amino-5-methylisoxazole

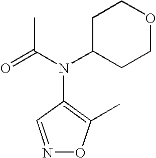

5-Methyl-4-amino-isoxazole (Reiter, L. A., J. Org. Chem. 1987, 52, 2714-2726) (0.68 g, 5.1 mmol) and acetic acid (0.61 g, 10.2 mmol) were dissolved in MeOH (20 mL). Tetrahydro-2H-pyran-4-one (0.76 g, 7.6 mmol) was added and the mixture was cooled to 0-(−5)° C. and stirred for 1 h. Sodium cyanoborohydride (0.32 g, 5.1 mmol) was added to the reaction mixture at −5° C., causing weak exothermic and gas evolution. The cooling bath was removed and the mixture was stirred at r.t. for 1 h, followed by the addition of a second portion of sodium cyanoborohydride (0.1 g, 1.6 mmol). After stirring for 2 h at r.t., the mixture was filtered and the filtrate was concentrated in vacuo. The residue was dissolved in toluene and re-concentrated. The residue was dissolved in THF (10 mL) and acetic anhydride (1.56 g, 15.3 mmol) was added. The resulting mixture was stirred overnight at r.t. then for 1 h at +50° C. The volatiles were removed in vacuo and the residue was dissolved in toluene and concentrated in vacuo to give the title compound (1.36 g, 78%).

¹H NMR (CDCl₃) ppm δ 8.04 (s, 1H), 4.86-4.73 (m, 1H), 4.00-3.89 (m, 2H), 3.52-3.42 (m, 2H), 2.35 (s, 3H), 1.81 (s, 3H), 1.70-1.57 (m, 2H), 1.49-1.23 (m, 2H); MS (ESI) m/z 225 (M+1).

### Example 6(b)

### 5-Acetyl-2-methyl-1-(tetrahydro-2H-pyran-4-yl)-1H-imidazole

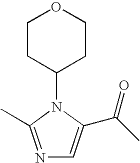

4-[N-Acetyl-N-(tetrahydro-2H-pyran-4-yl)]amino-5-methylisoxazole (4.8 g, 21.4 mmol) was dissolved in EtOH (30 ml), and the mixture was hydrogenated over Pd/C (10%, wet paste, 0.10 g) at 3 bar. The reaction mixture was stirred at 50° C. for 3 h. An additional amount of Pd/C (10%, wet paste, 0.15 g) was added and the mixture was continued stirring at +50° C. for 3 h. Sodium methoxide (1.70 g, 31.46 mmol) was added and the resulting mixture was heated to reflux for 30 h. Ammonium chloride was added to quench the reaction. The mixture was filtrated through diatomaceous earth and the filtrate was evaporated in vacuo. The residue was diluted with saturated sodium bicarbonate (aq.) and extracted with EtOAc, then with CHCl₃. The combined organic layers were dried (Na₂SO₄) and concentrated in vacuo. The crude product was purified by flash chromatography (EtOAc) to give the title compound (3.7 g, 83%).

¹H NMR (CDCl₃) δ 7.70 (s, 1H), 5.40-5.30 (m, 1H), 4.13-4.01 (m, 2H), 3.57-3.44 (m, 2H), 2.57 (s, 3H), 2.44 (s, 3H), 2.43-2.30 (m, 2H), 1.80-1.72 (m, 2H).

### Example 6(c)

### (2E)-3-Dimethylamino-1-[2-methyl-1-(tetrahydro-2H-pyran-4-yl)-1H-imidazol-5-yl]prop-2-en-1-one

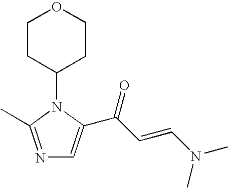

5-Acetyl-2-methyl-1-(tetrahydro-2H-pyran-4-yl)-1H-imidazole (3.7 g, 17.79 mmol) was dissolved in DMFDMA/DMF (1:1, 100 mL) and the mixture was stirred under reflux overnight. After cooling to r.t. the mixture was extracted with CH₂Cl₂. The organic phase was dried (Na₂SO₄), filtered and concentrated in vacuo. The crude product was purified by flash chromatography (CH₂Cl₂/MeOH 15:1) to give the title compound (3.85 g, 82%).

¹H NMR (CDCl₃) δ 7.65 (d, J=12.6 Hz, 1H), 7.46 (s, 1H), 5.55-5.42 (m, 2H), 4.08 (dd, J=11 Hz, 4.4 Hz, 2H), 3.52 (t, J=11 Hz, 2H), 2.99 (br s, 6H), 2.56 (s, 3H), 2.45-2.32 (m, 2H), 1.80-1.72 (m, 2H); MS (ESI) m/z 264 (M+1).

### Example 6(d)

### (2Z)-3-Dimethylamino-2-fluoro-1-[2-methyl-1-(tetrahydro-2H-pyran-4-yl)-1H-imidazol-5-yl]prop-2-en-1-one

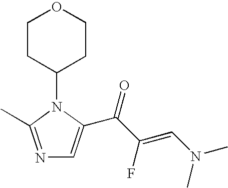

Selectfluor (7.75 g, 21.87 mmol) was added in portions to a stirred solution of (2E)-3-dimethylamino-1-[2-methyl-1-(tetrahydro-2H-pyran-4-yl)-1H-imidazol-5-yl]prop-2-en-1-one (3.85 g, 14.58 mmol) in MeOH (100 mL) at r.t. After stirring at r.t. for 3 h the reaction mixture was cooled in ice/acetone and filtered. The filtrate was evaporated under reduced pressure and the residue was taken into CH₂Cl₂. It was washed with aq. ammonia, brine, dried (Na₂SO₄) and concentrated in vacuo. The crude product was purified by flash chromatography (CH₂Cl₂/MeOH 15:1). The reaction was not run to completion, and the reaction was repeated again with Selectfluor (1.5 equiv.) followed by the same workup. The title compound (1.47 g, 36%).

¹H NMR (CDCl₃, 300 MHz) δ 7.34 (s, 1H), 6.84 (d, J=27.9 Hz, 1H), 5.00-4.88 (m, 1H), 4.04 (dd, J=11.2 Hz, 4.2 Hz, 2H), 3.46 (t, J=11 Hz, 2H), 3.08 (s, 6H), 2.53 (s, 3H), 2.42-2.28 (m, 2H), 1.84-1.75 (m, 2H); MS (ESI) m/z 282 (M⁺+1).

### Example 6(e)

### 5-Fluoro-4-[2-methyl-1-(tetrahydro-2H-pyran-4-yl)-1H-imidazol-5-yl]pyrimidin-2-amine

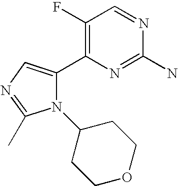

A reaction mixture of (2)-3-dimethylamino-2-fluoro-1-[2-methyl-1-(tetrahydro-2H-pyran-4-yl)-1H-imidazol-5-yl]prop-2-en-one (1.47 g, 5.22 mmol), guanidine carbonate (2.35 g, 13.06 mmol) and sodium methoxide (4.0 equiv.) in 1-butanol was heated in a microwave reactor for 10 minutes at 140° C. under argon or nitrogen atmosphere. The mixture was filtered and the filter was rinsed with CH₂Cl₂. The solvent was evaporated in vacuo and the crude product was purified using flash column chromatography (CH₂Cl₂/MeOH 20:1) to give the title compound (1.21 g, 84%).

¹H NMR (CDCl₃, 300 MHz) δ 8.17 (d, J=3.3 Hz, 1H), 7.59 (d, J=3.9 Hz, 1H), 5.27-5.13 (m, 1H), 4.93 (br s, 2H), 4.13 (dd, J=11.5 Hz, 4.3 Hz, 2H), 3.48 (t, J=11 Hz, 2H), 2.62 (s, 3H), 2.58-2.40 (m, 2H), 1.95-1.84 (m, 2H); MS (ESI) m/z 278 (M+1).

### Example 7

### 4-(1,2-Dimethyl-1H-imidazol-5-yl)-5-fluoropyrimidin-2-amine

### Example 7(a)

### 1,2-Dimethyl-5-(trimethylstannyl)-1N-imidazole

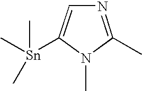

1,2-Dimethylimidazole (0.960 g, 10.0 mmol) was diluted in dry THF (50 mL) under an argon atmosphere and the solution was cooled to −78° C. tert-Butyllithium (1.7M in pentane, 6.47 mL, 11.0 mmol) was added dropwise over 5 minutes. The reaction mixture was stirred for 1 h at −78° C. and then treated with a solution of trimethyltin chloride (2.2 g, 11.0 mmol) in anhydrous THF (10 mL). The mixture was stirred for 60 h from −78° C. to r.t. The solvent was then evaporated in vacuo to give the title compound (1.29 g, 50%).

The crude product was used in the next step without further purification.

¹H NMR (CDCl₃) δ ppm 6.87 (s, 1H), 3.56 (s, 3H), 2.41 (s, 3H), 0.45-0.18 (m, 9H); MS (CI) m/z 261 (¹²⁰Sn) (M+1).

### Example 7(b)

### 2-Chloro-4-(1,2-dimethyl-1H-imidazol-5-yl)-5-fluoropyrimidine

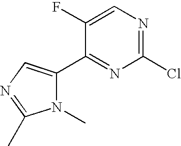

1,2-Dimethyl-5-(trimethylstannyl)-1H-imidazole (0.950 g, 3.68 mmol) and 2,4-dichloro-5-fluoropyrimidine (0.601 g, 3.60 mmol) were diluted in anhydrous DMF (20 mL) and the solution was degassed with argon. Pd(PPh₃)₂Cl₂ (0.126 g, 0.17 mmol) was added and the reaction mixture was stirred at +80° C. for 15 h. The reaction mixture was cooled down to r.t. and concentrated under reduced pressure. Saturated potassium fluoride (aq., 50 mL) was added and the mixture was stirred for 30 minutes before extraction with EtOAc. The organic layer was dried (MgSO₄), filtered and concentrated under reduced pressure. The crude product was purified by flash chromatography (heptane/EtOAc, 7:3) to give the title compound (0.41 g, 50%).

¹H NMR (CDCl₃, 600 MHz) δ ppm 8.40 (d, J=2.9 Hz, 1H), 7.86 (d, J=4.4 Hz, 1H), 3.97 (s, 3H), 2.53 (s, 3H); MS (ESI) m/z 227 (M+1).

### Example 7(c)

### 4-(1,2-Dimethyl-1H-imidazol-5-yl)-5-fluoropyrimidin-2-amine

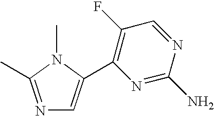

2-Chloro-4-(1,2-dimethyl-1H-imidazol-5-yl)-5-fluoropyrimidine (0.295 g, 1.30 mmol) was dissolved in 1-propanol (3.0 mL) in a microwave vial. Ammonium hydroxide (28%, 1.0 mL) was added, the vial was sealed and the mixture heated in a microwave oven (+140° C., 4 h). The reaction mixture was cooled to r.t. and the solvent was evaporated. The residue was partitioned between CH₂Cl₂ and 1M aqueous HCl. The aqueous phase, containing the product, was neutralized with saturated aqueous NaHCO₃ and the product extracted with CH₂Cl₂. The organic phase was co-evaporated with ethanol and the residue was purified by flash chromatography using (CH₂Cl₂/MeOH gradient; 100:1 to 94:6) to give the title compound (0.210 g, 78%).

¹H NMR (CDCl₃) δ ppm 8.15 (d, J=3.5 Hz, 1H), 7.71 (d, J=4.3 Hz, 1H), 4.87 (br s, 2H), 3.97 (s, 3H), 2.49 (s, 3H); MS (ESI) m/z 208 (M+1).

### Example 8

### 5-Fluoro-4-[1-(tetrahydro-2H-pyran-4-yl)-2-(trifluoromethyl)-1H-imidazol-5-yl]pyrimidin-2-amine

### Example 8(a)

### 5-Acetyl-1-(tetrahydro-2H-pyran-4-yl)-2-trifluoromethyl-1H-imidazole

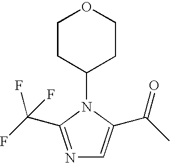

5-Methyl-4-amino-isoxazole (1.7 g, 17.25 mmol) and acetic acid (1.1 g, 19 mmol) were dissolved in methanol (50 mL). Tetrahydro-2H-pyran-4-one (1.9 g, 19 mmol) was added and the mixture was cooled to 0-(−5)° C. and stirred for 1 h. Sodium cyanoborohydride (0.812 g, 12.9 mmol) was added in portions to the reaction mixture at −5° C., causing weak exothermic and gas evolution. The cooling bath was removed and the mixture was stirred at r.t. for 2 h followed by addition of water (20 mL). The methanol was removed from the reaction mixture, and the intermediate amine was extracted with ethyl acetate (3×80 mL). The combined organic layers were dried (Na₂SO₄), concentrated to dryness, dissolved in toluene and re-concentrated. The crude intermediate amine, was dissolved in CH₂Cl₂ (20 mL) and pyridine (2 mL, 26 mmol) was added. The mixture was cooled to 0° C. and trifluoroacetic anhydride (4.35 g, 20.7 mmol) was added dropwise. The mixture was continued stirring for 2 h at r.t. and was then washed with water and saturated NaHCO₃. The aqueous layer was extracted with CH₂Cl₂ (2×30 mL), the organic extracts were dried (Na₂SO₄) and concentrated to dryness to give a second crude intermediate, 4-[N-(tetrahydro-2H-pyran-4-yl)]-N-trifluoroacetyl-amino-5-methylisoxazole. MS (ES) m/z 279 (M⁺+1). The title compound was prepared in accordance with the general method of Example 6 (b) using the intermediate 4-[N-(tetrahydro-2H-pyran-4-yl)]-N-trifluoroacetyl-amino-5-methylisoxazole (max 17.25 mmol), with the exception that the product was purified by flash chromatography (heptane/EtOAc 3:2), giving the title compound (3.03 g, 67%).

¹H NMR (CDCl₃, 300 MHz) δ 7.85 (s, 1H), 4.89-4.75 (m, 1H), 4.17-4.07 (m, 2H), 3.54-3.44 (m, 2H), 2.75-2.60 (m, 2H), 2.56 (s, 3H), 1.72-1.63 (m, 2H); MS (ES) m/z 263 (M+1).

### Example 8(b)

### (2E)-3-Dimethylamino-1-[1-(tetrahydro-2H-pyran-4-yl)-2-trifluoromethyl-1H-imidazol-5-yl]prop-2-en-1-one

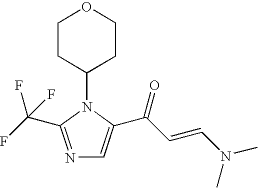

The title compound was prepared in accordance with the general method of Example 6(c) with the exception that the product was purified by flash chromatography (EtOAc). Using 5-acetyl-1-(tetrahydro-2H-pyran-4-yl)-2-trifluoromethyl-1H-imidazole (3.03 g, 11.55 mmol) the title compound was obtained (3.2 g, 87%).

¹H NMR (CDCl₃, 300 MHz) δ 7.72 (d, J=12.3 Hz, 1H), 7.49 (s, 1H), 5.50 (d, J=12.3 Hz, 1H), 4.89-4.75 (m, 1H), 4.14-4.05 (m, 2H), 3.54-3.44 (m, 2H), 3.16 (br. s, 3H), 2.93 (br. s, 3H), 2.86-2.72 (m, 2H), 1.80-1.72 (m, 2H); MS (ES) m/z 318 (M+1).

### Example 8(c)

### (2Z)-3-Dimethylamino-2-fluoro-1-[1-(tetrahydro-2H-pyran-4-yl)-2-trifluoromethyl-1H-imidazol-5-yl]prop-2-en-1-one

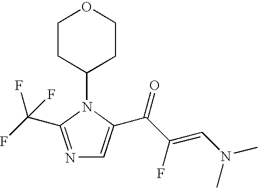

Selectfluor (0.370 g, 1.04 mmol) was added in portions to a stirred solution of (2E)-3-dimethylamino-1-[1-(tetrahydro-2H-pyran-4-yl)-2-trifluoromethyl-1H-imidazol-5-yl]prop-2-en-1-one (0.300 g, 0.946 mmol) in MeCN (20 mL) at 0° C. After stirring for 0.5 h at r.t. more Selectfluor (0.050 g, 0.14 mmol) was added, and the mixture was stirred for 0.5 h. The solvent was evaporated in vacuo, diluted with 3% aqueous NH₃ (20 mL) and extracted with CHCl₃ (3×20 mL). The organic extracts were dried (Na₂SO₄), evaporated in vacuo and the crude product was purified by flash chromatography (heptane/EtOAc 1:2), followed by neat EtOAc) to obtain the title compound (0.170 g, 53%).

¹H NMR (CDCl₃, 300 MHz) δ 7.34 (s, 1H), 6.85 (d, J=26.7 Hz, 1H), 4.67-4.54 (m, 1H), 4.11-4.03 (m, 2H), 3.50-3.38 (m, 2H), 3.14 (s, 6H), 2.72-2.56 (m, 2H), 1.83-1.74 (m, 2H); MS (ES) m/z 336 (M+1).

### Example 8(d)

### 5-Fluoro-4-[1-(tetrahydro-2H-pyran-4-yl)-2-(trifluoromethyl)-8H-imidazol-5-yl]pyrimidin-2-amine

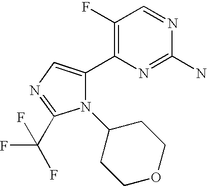

The title compound was prepared in accordance with the method in 6(e) by using (2Z)-3-dimethylamino-2-fluoro-1-[1-(tetrahydro-2H-pyran-4-yl)-2-trifluoromethyl-1H-imidazol-5-yl]prop-2-en-1-one (0.330 g, 1.0 mmol) and guanidine carbonate (0.45 g, 2.50 mmol). After purification by flash chromatography (heptane/EtOAc 1:2), the title compound was obtained (0.170 g, 51%).

¹H NMR (CDCl₃, 300 MHz) δ 8.29 (s, 1H), 7.63 (d, J=2.7 Hz, 1H), 5.10 (br.s., 2H), 4.88-4.76 (m, 1H), 4.16-4.07 (m, 2H), 3.53-3.42 (m, 2H), 2.80-2.65 (m, 2H), 1.89-1.81 (m, 2H); MS (ES) m/z 332 (M+1).

### Example 9

### 5-Fluoro-4-[1-methyl-2-(trifluoromethyl)-1H-imidazol-5-yl]pyrimidin-2-amine

### Example 9(a) 2,2,2-Trifluoro-N-methyl-N-(5-methylisoxazol-4-yl)acetamide

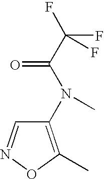

Trifluoroacetic anhydride (10 mL, 71 mmol) in CH₂Cl₂ (100 mL) was added to N,5-dimethylisoxazol-4-amine (Reiter, L. A., J. Org. Chem. 1987, 52, 2714-2726) (6.68 g, 59.6 mmol) in DCM (200 mL) and pyridine (6 mL, 74 mmol) at 0° C. The mixture was stirred at 0° C. for 30 min and at r.t. for 2 h. The reaction mixture was diluted with CH₂Cl₂ (100 mL) and washed with H₂O and saturated NaHCO₃ (aq). The organic layer was dried (Na₂SO₄), concentrated in vacuo to give the title compound (12.4 g, 100%).

MS (ESI) m/z 208 (M⁺).

### Example 9(b)

### 1-[1-Methyl-2-(trifluoromethyl)-1H-imidazol-5-yl]ethanone

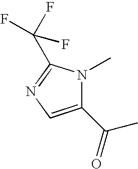

2,2,2-Trifluoro-N-methyl-N-(5-methylisoxazol-4-yl)acetamide (12.4 g, 59.6 mmol, obtained from Example 9(a)) in EtOH (30 ml) was hydrogenated over Pd/C (10%, 1.0 g) at 50 psi. The reaction mixture was stirred at +50° C. overnight. Sodium methoxide (5.0 g, 87.7 mmol) was added and the resulting mixture was heated to reflux overnight. The mixture was filtered through diatomaceous earth and the residue was diluted with saturated NaHCO₃ (aq.) and extracted with EtOAc. The combined organic layers were dried (Na₂SO₄) and concentrated in vacuo. The crude product was purified by flash chromatography (Heptane:EtOAc 2:1) to give the title compound (6.1 g, 52%).

¹H NMR (400 MHz, CDCl₃) δ ppm 7.77 (s, 1H), 4.07 (s, 3H), 2.54 (s, 3H);

MS (ESI) m/z 192 (M⁺).

### Example 9(c)

### (2E)-3-(Dimethylamino)-1-[1-methyl-2-(trifluoromethyl)-1H-imidazol-5-yl]prop-2-en-1-one

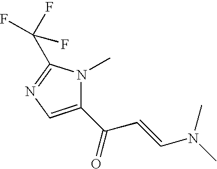

1-[1-Methyl-2-(trifluoromethyl)-1H-imidazol-5-yl]ethanone (6.0 g, 31 mmol, obtained from Example 9(b)) was dissolved in DMFDMA/DMF (1:1, 46 mL) and the mixture was stirred at +100° C. overnight. After cooling to r.t. the mixture was diluted with H₂O and extracted with CH₂Cl₂ (three times). The organic phases were combined, dried (Na₂SO₄), filtered and concentrated in vacuo to give the title compound (7.11 g, 93%).

MS (ESI) m/z 247 (M⁺); MS (ESI) m/z 248 (M+1).

### Example 9(d)

### (2Z)-3-(Dimethylamino)-2-fluoro-1-[1-methyl-2-(trifluoromethyl)-1H-imidazol-5-yl]prop-2-en-1-one

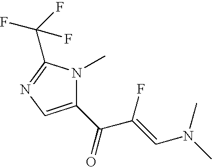

Selectfluor (10.9 g, 30.8 mmol) was added in portions to a stirred solution of (2E)-3-(dimethylamino)-1-[1-methyl-2-(trifluoromethyl)-1H-imidazol-5-yl]prop-2-en-1-one (7.0 g, 28.3 mmol, obtained from Example 9(c)) in CH₃CN (250 mL) at 0° C. After stirring at 0° C. for 1.5 h the reaction mixture was diluted with H₂O and extracted with CH₂Cl₂ (three times). The organic phases were combined, dried (Na₂SO₄), filtered and concentrated in vacuo to give the crude title compound that was used in the next step without any further purification. MS (ESI) m/z 265 (M⁺); MS (ESI) m/z 266 (M+1).

### Example 9(e)

### 5-Fluoro-4-[1-methyl-2-(trifluoromethyl)-1H-imidazol-5-yl]pyrimidin-2-amine

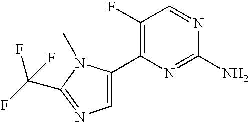

A reaction mixture of (2)-3-(dimethylamino)-2-fluoro-1-[1-methyl-2-(trifluoromethyl)-1H-imidazol-5-yl]prop-2-en-1-one (28.3 mmol, crude from Example 9(d)), guanidine is carbonate (13.5 g, 75 mmol) and NaOMe (6.5 g, 120 mmol) in 1-butanol (250 mL) was heated to reflux under argon atmosphere for 2.5 h. The mixture was diluted with H₂O and extracted with CH₂Cl₂. The organic phases were combined, dried (Na₂SO₄), filtered and concentrated in vacuo. The crude product was purified by flash chromatography (Heptane:EtOAc 1:1 to Heptane:EtOAc 1:2) to give the title compound (1.76 g, 21%).

¹H NMR (400 MHz, CDCl₃) δ ppm 8.27 (d, J=3.03 Hz, 1H) 7.74 (d, J=4.04 Hz, 1H) 5.02 (br. s., 2H) 4.14 (s, 3H); MS (ESI) m/z 261 (M⁺).

### Example 10

### (6-Ethoxy-pyridin-3-yl)-{5-fluoro-4-[2-methyl-3-(tetrahydro-pyran-4-yl)-3H-imidazol-4-yl]-pyrimidin-2-yl}-amine

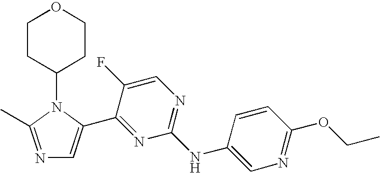

The title compound was prepared in accordance with the general method A using 5-fluoro-4-[2-methyl-1-(tetrahydro-2H-pyran-4-yl)-1H-imidazol-5-yl]pyrimidin-2-amine (as described in Example 6) (50 mg, 0.18 mmol) and 5-bromo-2-ethoxy-pyridine (36 mg, 0.18 mmol) to give the title compound (27 mg, 38%).

¹H NMR (CDCl₃) δ ppm 8.24 (m, 2H) 7.68 (m, 1H) 7.56 (m, 1H) 7.36 (br s, 1H) 6.70 (d, J=8.84 Hz, 1H) 5.11 (m, 1H) 4.32 (q, J=7.07, 2H) 3.95-3.91 (m, 2H) 3.05 (m, 2H) 2.61 (s, 3H) 2.35-2.24 (m, 2H) 1.75 (m, 2H), 1.39 (t, J=7.07 Hz, 3H); MS (ES) m/z 399 (M+1).

### Example 11

### {5-Fluoro-4-[2-methyl-3-(tetrahydro-pyran-4-yl)-3H-imidazol-4-yl]-pyrimidin-2-yl}-(2-methoxy-pyrimidin-5-yl)-amine

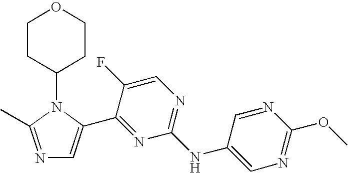

The title compound was prepared in accordance with the general method A using 5-fluoro-4-[2-methyl-1-(tetrahydro-2H-pyran-4-yl)-1H-imidazol-5-yl]pyrimidin-2-amine (as described in Example 6) (50 mg, 0.18 mmol) and 5-bromo-2-methoxy-pyrimidine (34 mg, 0.18 mmol) to give the title compound (8 mg, 12%).

¹H NMR (CDCl₃) δ ppm 8.70 (s, 2H) 8.29 (m, 1H) 7.62 (d, J=4.04 Hz, 1H) 7.11 (s, 1H) 5.06 (m, 1H) 4.03 (m, 1H) 4.01 (s, 3H) 3.17 (m, 2H) 2.63 (s, 3H) 2.41 (m, 2H) 1.81 (m, 2H); MS (ES) m/z 386 (M+1).

### Examples 12-40

The following Examples 12-40 were prepared by the general procedure B using the appropriate starting materials which include: lithium 5-[[5-fluoro-4-[2-methyl-3-(oxan-4-yl)imidazol-4-yl]pyrimidin-2-yl]amino]pyridine-2-carboxylate (as described below) and the amine necessary to deliver the amide shown in the table below.

### Lithium 5-[[5-fluoro-4-[2-methyl-3-(oxan-4-yl)imidazol-4-yl]pyrimidin-2-yl]amino]pyridine-2-carboxylate

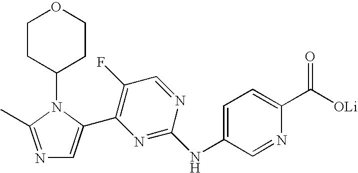

Methyl 5-[[5-fluoro-4-[2-methyl-3-(oxan-4-yl)imidazol-4-yl]pyrimidin-2-yl]amino]pyridine-2-carboxylate (prepared as described in Example 41) (1.49 g, 3.61 mmol) in MeOH (70 mL) was heated at 60° C. for 30 min. The flask was removed from the oil bath and a solution of LiOH monohydrate (167 mg, 3.97 mmol) in water (13 mL) was added dropwise during one minute. The mixture was heated at 60° C. during 4 h, allowed to cool and concentrated to a yellow powder which was dried under vacuum to yield 1.44 g (99%) of the title compound. The isolated material was used in amidation reactions without further purification.

¹H NMR (400 MHz, DMSO-d₆) δ ppm 8.52 (d, 1H), 8.02 (d, 1H), 7.83 (d, 1H), 7.32 (d, 1H), 5.08-4.99 (m, 1H), 3.82-3.78 (m, 2H), 3.06 (t, 2H), 2.56 (s, 3H), 2.22-2.14 (m, 2H), 1.79-1.77 (m, 2H); MS (ESI) m/z 399 (M+1).

### Example 12

### N-Butan-2-yl-5-[[5-fluoro-4-[2-methyl-3-(oxan-4-yl)imidazol-4-yl]pyrimidin-2-yl]amino]-N-propyl-pyridine-2-carboxamide

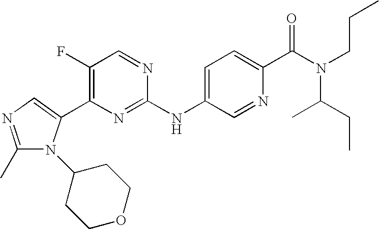

Amine: N-propylbutan-2-amine

Yield: 56%

m/z* (M+1): 496

NMR: 9.90 (s, 1H), 8.75-8.80 (m, 1H), 8.64 (d, 1H), 8.08-8.18 (m, 1H), 7.40-7.48 (m, 1H), 7.36 (d, 1H), 4.93-5.05 (m, 1H), 4.15-4.25 (m, 0.5H), 3.80-3.87 (m, 2H), 3.70-3.79 (m, 0.5H), 3.09-3.19 (m, 2H), 2.98-3.08 (m, 1H), 2.54 (s, 3H), 2.11-2.25 (m, 2H), 1.80 (d, 2H), 1.30-1.74 (m, 4H), 1.17-1.26 (m, 1H), 1.14 (d, 2H), 0.84-0.95 (m, 3H), 0.68 (t, 2H), 0.61 (t, 1H).

### Example 13

### (3,3-Difluoropyrrolidin-1-yl)-[5-[[5-fluoro-4-[2-methyl-3-(oxan-4-yl)imidazol-4-yl]pyrimidin-2-yl]amino]pyridin-2-yl]methanone

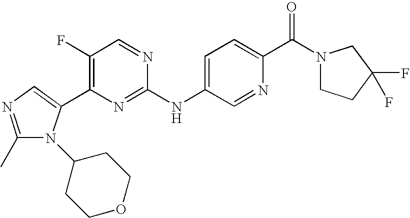

Amine: 3,3-Difluoropyrrolidine

Yield: 62%

m/z* (M+1): 488

NMR: 10.08 (s, 0.6H), 10.06 (s, 0.4H), 8.85-8.91 (m, 1H), 8.67 (d, 1H), 8.18 (dd, 1H), 7.86 (d, 0.6H), 7.81 (d, 0.4H), 7.37 (d, 1H), 4.93-5.07 (m, 1H), 4.28 (t, 1H), 4.05 (t, 1H), 3.92 (t, 1H), 3.83 (dd, 2H), 3.75 (t, 1H), 3.14 (t, 2H), 2.55 (s, 3H), 2.35-2.48 (m, 2H), 2.12-2.27 (m, 2H), 1.81 (d, 2H).

### Example 14

### [5-[[5-Fluoro-4-[2-methyl-3-(oxan-4-yl)imidazol-4-yl]pyrimidin-2-yl]amino]pyridin-2-yl]-(3-methyl-1-piperidyl)methanone

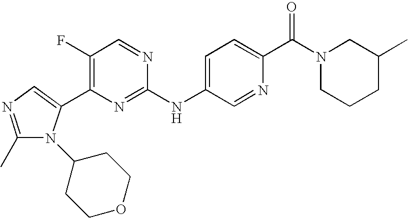

Amine: 3-Methylpiperidine

Yield: 68%

m/z* (M+1): 480

NMR: 9.93 (s, 1H), 8.79 (s, 1H), 8.64 (d, 1H), 8.08-8.15 (m, 1H), 7.51 (d, 1H), 7.36 (d, 1H), 4.93-5.05 (m, 1H), 4.23-4.36 (m, 1H), 3.78-3.87 (m, 2.5H), 3.69-3.79 (m, 1H), 3.06-3.17 (m, 2H), 2.94-3.05 (m, 0.5H), 2.64-2.84 (m, 1H), 2.54 (s, 3H), 2.11-2.24 (m, 2H), 1.75-1.83 (m, 3H), 1.53-1.73 (m, 2H), 1.36-1.49 (m, 1H), 1.11-1.22 (m, 1H), 0.92 (d, 1.5H), 0.74 (d, 1.5H)

### Example 15

### 5-[[5-Fluoro-4-[2-methyl-3-(oxan-4-yl)imidazol-4-yl]pyrimidin-2-yl]amino]-N-methyl-N-propan-2-yl-pyridine-2-carboxamide

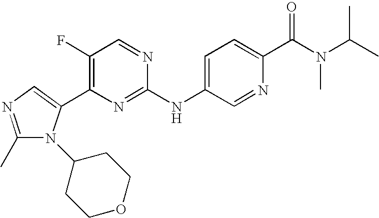

Amine: N-Methylpropan-2-amino

Yield: 39%

m/z* (M+1): 454

NMR: 9.92 (s, 1H), 8.79 (s, 1H), 8.64 (d, 1H), 8.06-8.16 (m, 1H), 7.44-7.54 (m, 1H), 7.36 (d, 1H), 4.92-5.03 (m, 1H), 4.66-4.77 (m, 0.4H), 3.99-4.09 (m, 0.6H), 3.83 (dd, 2H), 3.12 (t, 2H), 2.82 (s, 2H), 2.80 (s, 1H), 2.54 (s, 3H), 2.11-2.25 (m, 2H), 1.79 (d, 2H), 1.08-1.18 (m, 6H).

### Example 16

### [5-[[5-Fluoro-4-[2-methyl-3-(oxan-4-yl)imidazol-4-yl]pyrimidin-2-yl]amino]pyridin-2-yl]-[4-(4-fluorophenyl)-1-piperidyl]methanone

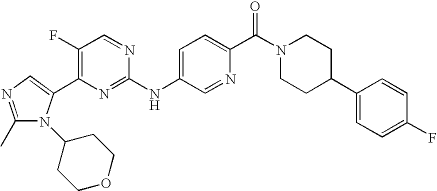

Amine: 4-(4-Fluorophenyl)piperidine

Yield: 62%

m/z* (M+1): 560

NMR: 9.95 (s, 1H), 8.80 (d, 1H), 8.64 (d, 1H), 8.13 (dd, 1H), 7.57 (d, 1H), 7.36 (d, 1H), 7.30 (dd, 2H), 7.11 (t, 2H), 4.93-5.03 (m, 1H), 4.59-4.69 (m, 1H), 3.98-4.07 (m, 1H), 3.82 (dd, 2H), 3.06-3.21 (m, 3H), 2.79-2.91 (m, 2H), 2.54 (overlap. s, 3H), 2.12-2.24 (m, 2H), 1.69-1.92 (m, 4H), 1.52-1.66 (m, 2H).

### Example 17

### (4-Ethylpiperazin-1-yl)-[5-[[5-fluoro-4-[2-methyl-3-(oxan-4-yl)imidazol-4-yl]pyrimidin-2-yl]amino]pyridin-2-yl]methanone

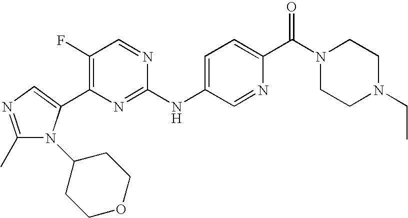

Amine: 1-Ethylpiperazine

Yield: 77%

m/z* (M+1): 495

NMR: 9.96 (s, 1H), 8.80 (d, 1H), 8.65 (d, 1H), 8.13 (dd, 1H), 7.55 (d, 1H), 7.36 (d, 1H), 4.93-5.03 (m, 1H), 3.82 (dd, 2H), 3.62 (br. s., 2H), 3.53 (br. s., 2H), 3.12 (t, 2H), 2.55 (s, 3H), 2.41 (br. s., 2H), 2.30-2.38 (m, 4H), 2.11-2.24 (m, 2H), 1.79 (dd, 2H), 1.00 (t, 3H).

### Example 18

### (4-Butylpiperazin-1-yl)-[5-[[5-fluoro-4-[2-methyl-3-(oxan-4-yl)imidazol-4-yl]pyrimidin-2-yl]amino]pyridin-2-yl]methanone

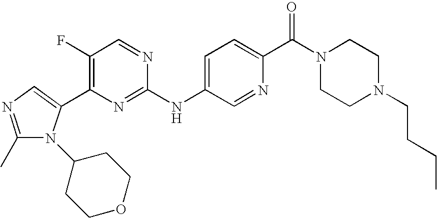

Amine: 1-Butylpiperazine

Yield: 64%

m/z* (M+1): 523

NMR: 9.96 (s, 1H), 8.80 (d, 1H), 8.65 (d, 1H), 8.12 (dd, 1H), 7.55 (d, 1H), 7.36 (d, 1H), 4.93-5.03 (m, 1H), 3.82 (dd, 2H), 3.62 (br. s., 2H), 3.52 (br. s., 2H), 3.12 (t, 2H), 2.55 (s, 3H), 2.41 (br. s., 2H), 2.33 (br. s., 2H), 2.25-2.31 (m, 2H), 2.11-2.24 (m, 2H), 1.79 (dd, 2H), 1.22-1.46 (m, 4H), 0.88 (t, 3H).

### Example 19

### N-Ethyl-5-[[5-fluoro-4-[2-methyl-3-(oxan-4-yl)imidazol-4-yl]pyrimidin-2-yl]amino]-N-propan-2-yl-pyridine-2-carboxamide

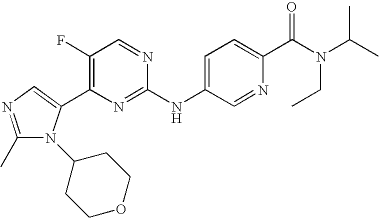

Amine: N-Ethylpropan-2-amine

Yield: 59%

m/z* (M+1): 468

NMR: 9.91 (s, 1H), 8.78 (d, 1H), 8.64 (d, 1H), 8.11 (d, 1H), 7.45 (d, 1H), 7.37 (d, 1H), 4.92-5.04 (m, 1H), 3.98-4.09 (m, 1H), 3.83 (dd, 2H), 3.13 (t, 2H), 2.55 (s, 3H), 2.11-2.26 (m, 2H), 1.79 (d, 2H), 1.08-1.27 (m, 9H), 0.99 (t, 1H).

### Example 20

### [5-[[5-Fluoro-4-[2-methyl-3-(oxan-4-yl)imidazol-4-yl]pyrimidin-2-yl]amino]pyridin-2-yl]-(1-piperidyl)methanone

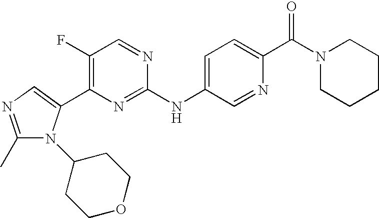

Amine: Piperidine

Yield: 50%

m/z* (M+1): 466

NMR: 9.93 (s, 1H), 8.79 (s., 1H), 8.62-8.67 (m, 1H), 8.08-8.15 (m, 1H), 7.50 (d, 1H), 7.36 (d, 1H), 4.92-5.04 (m, 1H), 3.78-3.87 (m, 2H), 3.58 (br. s., 2H), 3.42 (br. s., 2H), 3.12 (t, 2H), 2.54 (s, 3H), 2.10-2.25 (m, 2H), 1.79 (d, 2H), 1.58-1.66 (m, 2H), 1.55 (br. s., 2H), 1.48 (br. s., 2H).

### Example 21

### [5-[[5-Fluoro-4-[2-methyl-3-(oxan-4-yl)imidazol-4-yl]pyrimidin-2-yl]amino]pyridin-2-yl]-(4-propan-2-ylpiperazin-1-yl)methanone

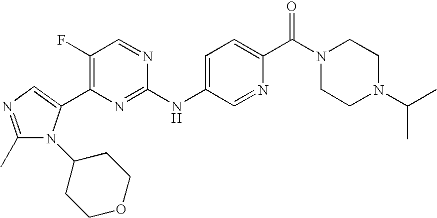

Amine: 1-Propan-2-ylpiperazine

Yield: 100%

m/z* (M+1): 509

NMR: 9.95 (s, 1H), 8.80 (d, 1H), 8.64 (d, 1H), 8.12 (dd, 1H), 7.55 (d, 1H), 7.36 (d, 1H), 4.92-5.04 (m, 1H), 3.82 (dd, 2H), 3.61 (br. s., 2H), 3.50 (br. s., 2H), 3.12 (t, 2H), 2.63-2.72 (m, 1H), 2.55 (s, 3H), 2.41 (br. s., 2H), 2.11-2.25 (m, 2H), 1.75-1.84 (m, 2H), 0.97 (d, 6H).

### Example 22

### 5-[[5-Fluoro-4-[2-methyl-3-(oxan-4-yl)imidazol-4-yl]pyrimidin-2-yl]amino]-N,N-dipropan-2-yl-pyridine-2-carboxamide

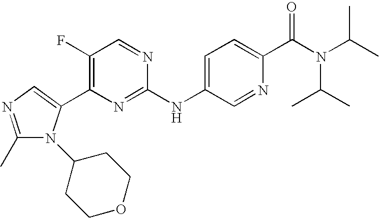

Amine: N-Propan-2-ylpropan-2-amine

Yield: 44%

m/z* (M+1): 482

NMR: 9.87 (s, 1H), 8.74 (d, 1H), 8.63 (d, 1H), 8.09 (dd, 1H), 7.39 (d, 1H), 7.35 (d, 1H), 4.92-5.04 (m, 1H), 3.81-3.84 (m, 3H), 3.57 (br. s., 1H), 3.12 (t, 2H), 2.54 (s, 3H), 2.11-2.25 (m, 2H), 1.79 (dd, 2H), 1.43 (br. s., 6H), 1.12 (br. s., 6H).

### Example 23

### (2,6-Dimethyl-1-piperidyl)-[5-[[5-fluoro-4-[2-methyl-3-(oxan-4-yl)imidazol-4-yl]pyrimidin-2-yl]amino]pyridin-2-yl]methanone

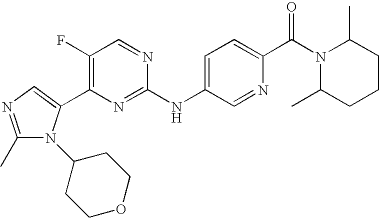

Amine: 2,6-Dimethylpiperidine

Yield: 39%

m/z* (M+1): 494

NMR: 9.89 (s, 1H), 8.77 (d, 1H), 8.64 (d, 1H), 8.11 (dd, 1H), 7.43 (d, 1H), 7.36 (d, 1H), 4.91-5.06 (m, 1H), 4.38 (br. s., 2H), 3.76-3.90 (m, 2H), 3.13 (t, 2H), 2.54 (s, 3H), 2.10-2.26 (m, 2H), 1.73-1.88 (m, 3H), 1.39-1.68 (m, 5H), 1.22 (d, 6H).

### Example 24

### 5-[[5-Fluoro-4-[2-methyl-3-(oxan-4-yl)imidazol-4-yl]pyrimidin-2-yl]amino]-N,N-dipropyl-pyridine-2-carboxamide

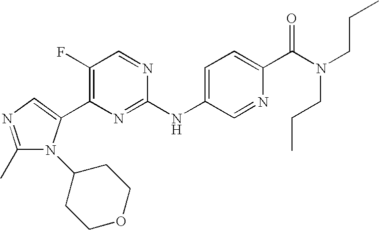

Amine: N-Propylpropan-1-amine

Yield: 69%

m/z* (M+1): 482

NMR: 9.92 (s, 1H), 8.77 (d, 1H), 8.64 (d, 1H), 8.14 (dd, 1H), 7.49 (d, 1H), 7.36 (d, 1H), 4.94-5.04 (m, 1H), 3.83 (dd, 2H), 3.34-3.40 (m, 4H), 3.13 (t, 2H), 2.54 (s, 3H), 2.11-2.25 (m, 2H), 1.80 (d, 2H), 1.46-1.66 (m, 4H), 0.90 (t, 3H), 0.68 (t, 3H).

### Example 25

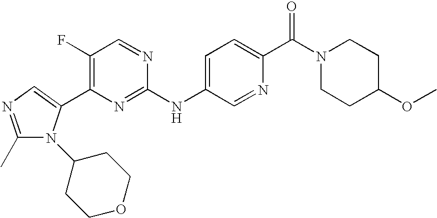

### [5-[[5-Fluoro-4-[2-methyl-3-(oxan-4-yl)imidazol-4-yl]pyrimidin-2-yl]amino]pyridin-2-yl]-(4-methoxy-1-piperidyl)methanone

Amine: 4-Methoxypiperidine

Yield: 68%

m/z* (M+1): 496

NMR: 9.95 (s, 1H), 8.80 (d, 1H), 8.64 (d, 1H), 8.12 (dd, 1H), 7.54 (d, 1H), 7.36 (d, 1H), 4.93-5.04 (m, 1H), 3.95 (br. s., 1H), 3.82 (dd, 2H), 3.67 (br. s., 1H), 3.39-3.49 (m, 2H), 3.26 (s, 3H), 3.12 (t, 2H), 2.55 (s, 3H), 2.11-2.25 (m, 2H), 1.74-1.96 (m, 4H), 1.44 (br. s., 2H).

### Example 26

### N-Ethyl-5-[[5-fluoro-4-[2-methyl-3-(oxan-4-yl)imidazol-4-yl]pyrimidin-2-yl]amino]-N-methyl-pyridine-2-carboxamide

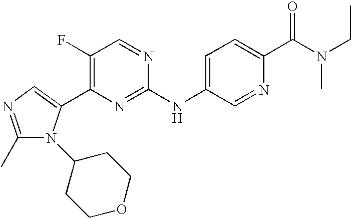

Amine: N-Methylethanamine

Yield: 67%

m/z* (M+1): 440

NMR: 9.93 (s, 1H), 8.79 (s, 1H), 8.64 (d, 1H), 8.09-8.15 (m, 1H), 7.52 (dd, 1H), 7.36 (d, 1H), 4.94-5.04 (m, 1H), 3.83 (dd, 2H), 3.46 (q, 1H), 3.35-3.40 (overlap. m, 1H), 3.13 (t, 2H), 2.99 (s, 1.5H), 2.95 (s, 1.5H), 2.54 (s, 3H), 2.12-2.25 (m, 2H), 1.75-1.84 (m, 2H), 1.12 (q, 3H).

### Example 27

### [5-[[5-Fluoro-4-[2-methyl-3-(oxan-4-yl)imidazol-4-yl]pyrimidin-2-yl]amino]pyridin-2-yl]-(4-methyl-1-piperidyl)methanone

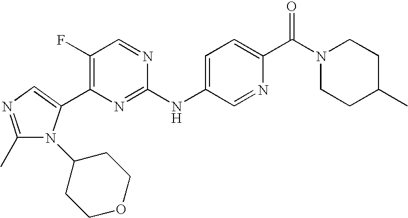

Amine: 4-Methylpiperidine

Yield: 75%

m/z* (M+1): 480

NMR: 9.93 (s, 1H), 8.78 (d, 1H), 8.64 (d, 1H), 8.11 (dd, 1H), 7.50 (d, 1H), 7.36 (d, 1H), 4.93-5.03 (m, 1H), 4.44 (d, 1H), 3.77-3.88 (m, 3H), 3.07-3.17 (m, 2H), 3.01 (t, 1H), 2.69-2.80 (m, 1H), 2.54 (s, 3H), 2.11-2.24 (m, 2H), 1.79 (d, 2H), 1.51-1.74 (m, 3H), 1.02-1.15 (m, 2H), 0.92 (d, 3H).

### Example 28

### (4-Benzylpiperazin-1-yl)-[5-[[5-fluoro-4-[2-methyl-3-(oxan-4-yl)imidazol-4-yl]pyrimidin-2-yl]amino]pyridin-2-yl]methanone

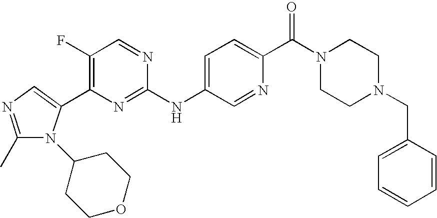

Amine: 1-Benzylpiperazine

Yield: 66%

m/z* (M+1): 557

NMR: 9.95 (s, 1H), 8.79 (d, 1H), 8.64 (d, 1H), 8.12 (dd, 1H), 7.55 (d, 1H), 7.36 (d, 1H), 7.22-7.34 (m, 5H), 4.93-5.03 (m, 1H), 3.82 (dd, 2H), 3.63 (br. s., 2H), 3.54 (br. s., 2H), 3.51 (s, 2H), 3.11 (t, 2H), 2.54 (s, 3H), 2.42 (br. s., 2H), 2.36 (br. s., 2H), 2.11-2.24 (m, 2H), 1.75-1.83 (m, 2H).

### Example 29

### (4,4-Difluoro-1-piperidyl)-[5-[[5-fluoro-4-[2-methyl-3-(oxan-4-yl)imidazol-4-yl]pyrimidin-2-yl]amino]pyridin-2-yl]methanone

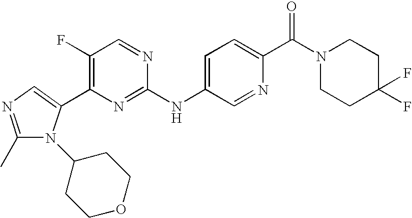

Amine: 4,4-Difluoropiperidine

Yield: 56%

m/z* (M+1): 502

NMR: 9.99 (s, 1H), 8.82 (d, 1H), 8.65 (d, 1H), 8.15 (dd, 1H), 7.63 (d, 1H), 7.36 (d, 1H), 4.92-5.05 (m, 1H), 3.83 (dd, 2H), 3.74 (br. s., 2H), 3.67 (br. s., 2H), 3.13 (t, 2H), 2.55 (s, 3H), 2.12-2.25 (m, 2H), 2.04 (br. s., 4H), 1.79 (d, 2H).

### Example 30

### N-Benzyl-5-[[5-fluoro-4-[2-methyl-3-(oxan-4-yl)imidazol-4-yl]pyrimidin-2-yl]amino]-N-propan-2-yl-pyridine-2-carboxamide

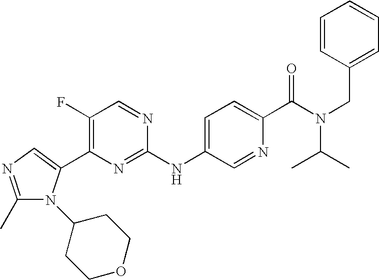

Amine: N-Benzylpropan-2-amine

Yield: 61%

m/z* (M+1): 530

NMR: 9.95 (s, 0.7H), 9.88 (br. s., 0.3H), 8.85 (br. s., 0.7H), 8.72 (br. s., 0.3H), 8.65 (br. s., 0.7H), 8.62 (br. s., 0.3H), 8.14 (d, 0.7H), 8.05 (d, 0.3H), 7.49-7.59 (m, 1H), 7.13-7.39 (m, 6H), 4.88-5.06 (m, 1H), 4.69 (br. s., 0.6H), 4.63 (s, 1.4H), 4.39-4.50 (m, 0.3H), 4.15-4.27 (m, 0.7H), 3.72-3.88 (m, 2H), 3.04-3.20 (m, 2H), 2.54 (s, 3H), 2.08-2.26 (m, 2H), 1.68-1.85 (m, 2H), 1.14 (d, 2H), 1.08 (d, 4H).

### Example 31

### 5-[[5-Fluoro-4-[2-methyl-3-(oxan-4-yl)imidazol-4-yl]pyrimidin-2-yl]amino]-N-methyl-N-(2-methylpropyl)pyridine-2-carboxamide

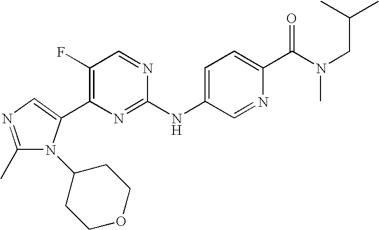

Amine: N,2-Dimethylpropan-1-amine

Yield: 74%

m/z* (M+1): 468

NMR: 9.93 (s, 1H), 8.80 (d, 1H), 8.64 (s, 1H), 8.09-8.17 (m, 1H), 7.50 (dd, 1H), 7.36 (d, 1H), 4.94-5.05 (m, 1H), 3.78-3.87 (m, 2H), 3.26-3.30 (overlap. m, 2H), 3.08-3.19 (m, 2H), 2.97 (s, 3H), 2.55 (br. s., 3H), 2.11-2.24 (m, 2H), 1.98-2.08 (m, 0.5H), 1.84-1.92 (m, 0.5H), 1.80 (d, 2H), 0.91 (d, 3H), 0.69 (d, 3H).

### Example 32

### [5-[[5-Fluoro-4-[2-methyl-3-(oxan-4-yl)imidazol-4-yl]pyrimidin-2-yl]amino]pyridin-2-yl]-(4-fluoro-1-piperidyl)methanone

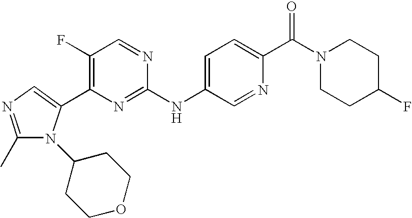

Amine: 4-Fluoropiperidine

Yield: 44%

m/z* (M+1): 484

NMR: 9.96 (s, 1H), 8.81 (d, 1H), 8.65 (d, 1H), 8.13 (dd, 1H), 7.57 (d, 1H), 7.36 (d, 1H), 4.93-5.04 (m, 1.5H), 4.83-4.90 (m, 0.5H), 3.83 (dd, 2H), 3.43-3.75 (m, 4H), 3.13 (t, 2H), 2.55 (s, 3H), 2.12-2.25 (m, 2H), 1.65-2.02 (m, 6H).

### Example 33

### N-Benzyl-N-ethyl-5-[[5-fluoro-4-[2-methyl-3-(oxan-4-yl)imidazol-4-yl]pyrimidin-2-yl]amino]pyridine-2-carboxamide

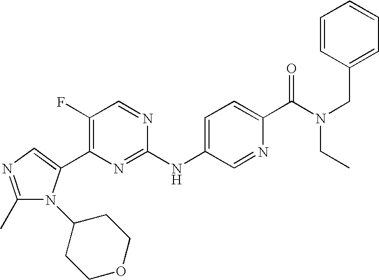

Amine: N-Benzylethanamine

Yield: 71%

m/z* (M+1): 516

NMR: 9.96 (br. s., 0.6H), 9.95 (br. s., 0.4H), 8.82-8.85 (m, 0.6H), 8.78-8.80 (m, 0.4H), 8.62-8.66 (m, 1H), 8.09-8.17 (m, 1H), 7.58-7.64 (m, 1H), 7.25-7.38 (m, 6H), 4.91-5.03 (m, 1H), 4.71 (br. s., 1H), 4.69 (s, 1H), 3.73-3.87 (m, 2H), 3.33-3.37 (m, 2H), 3.03-3.19 (overlap. m, 2H), 2.55 (br. s., 3H), 2.07-2.24 (m, 2H), 1.71-1.84 (m, 2H), 1.07 (t, 3H).

### Example 34

### (4-Butan-2-ylpiperazin-1-yl)-[5-[[5-fluoro-4-[2-methyl-3-(oxan-4-yl)imidazol-4-yl]pyrimidin-2-yl]amino]pyridin-2-yl]methanone

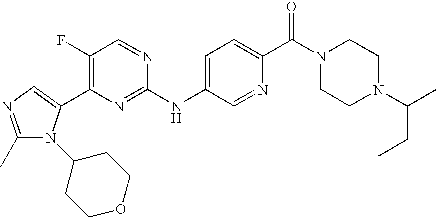

Amine: 1-Butan-2-ylpiperazine

Yield: 68%

m/z* (M+1): 523

NMR: 9.95 (s, 1H), 8.80 (d, 1H), 8.64 (d, 1H), 8.12 (dd, 1H), 7.55 (d, 1H), 7.36 (d, 1H), 4.93-5.04 (m, 1H), 3.82 (dd, 2H), 3.60 (br. s., 2H), 3.49 (br. s., 2H), 3.12 (t, 2H), 2.55 (s, 3H), 2.31-2.48 (m, 4H), 2.11-2.25 (m, 2H), 1.79 (d, 2H), 1.41-1.55 (m, 1H), 1.20-1.32 (m, 2H), 0.90 (d, 3H), 0.86 (t, 3H).

### Example 35

### (N-(Cyclopropylmethyl)-5-[[5-fluoro-4-[2-methyl-3-(oxan-4-yl)imidazol-4-yl]pyrimidin-2-yl]amino]-N-propyl-pyridine-2-carboxamide

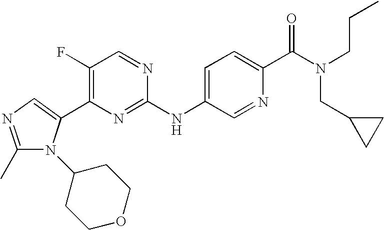

Amine: N-(Cyclopropylmethyl)propan-1-amine

Yield: 73%

m/z* (M+1): 494

Ret T.: 0.99.

### Example 36

### [5-[[5-Fluoro-4-[2-methyl-3-(oxan-4-yl)imidazol-4-yl]pyrimidin-2-yl]amino]pyridin-2-yl]-[4-(4-fluorophenyl)piperazin-1-yl]methanone

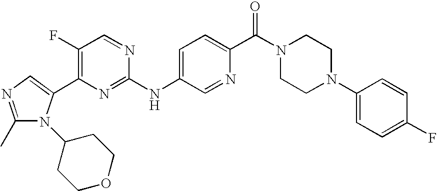

Amine: 1-(4-Fluorophenyl)piperazine

Yield: 75%

m/z* (M+1): 561

NMR: 9.98 (s, 1H), 8.83 (d, 1H), 8.65 (d, 1H), 8.15 (dd, 1H), 7.62 (d, 1H), 7.36 (d, 1H), 7.06 (t, 2H), 6.94-7.02 (m, 2H), 4.92-5.04 (m, 1H), 3.80-3.87 (m, 2H), 3.78 (br. s, 2H), 3.73 (br.s, 2H), 3.04-3.20 (m, 6H), 2.55 (s, 3H), 2.11-2.26 (m, 2H), 1.80 (d, 2H).

### Example 37

### [5-[[5-Fluoro-4-[2-methyl-3-(oxan-4-yl)imidazol-4-yl]pyrimidin-2-yl]amino]pyridin-2-yl]-(4-propylpiperazin-1-yl)methanone

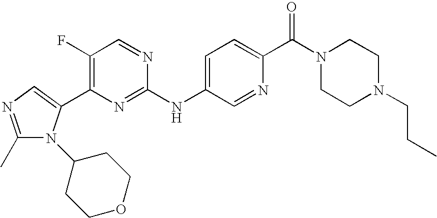

Amine: 1-Propylpiperazine

Yield: 93%

m/z* (M+1): 509

NMR: 9.96 (s, 1H), 8.80 (d, 1H), 8.65 (d, 1H), 8.12 (dd, 1H), 7.55 (d, 1H), 7.36 (d, 1H), 4.93-5.04 (m, 1H), 3.78-3.86 (m, 2H), 3.62 (br. s., 2H), 3.52 (br. s., 2H), 3.12 (t, 2H), 2.55 (s, 3H), 2.41 (br. s., 2H), 2.33 (br. s., 2H), 2.22-2.28 (m, 2H), 2.11-2.22 (m, 2H), 1.75-1.83 (m, 2H), 1.38-1.50 (m, 2H), 0.86 (t, 3H).

### Example 38

### N,N-Diethyl-5-[[5-fluoro-4-[2-methyl-3-(oxan-4-yl)imidazol-4-yl]pyrimidin-2-yl]amino]pyridine-2-carboxamide

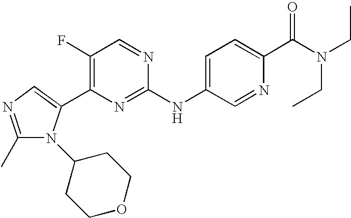

Amine: N-Ethylethanamine

Yield: 60%

m/z* (M+1): 454

NMR: 9.92 (s, 1H), 8.79 (d, 1H), 8.65 (d, 1H), 8.13 (dd, 1H), 7.52 (d, 1H), 7.36 (d, 1H), 4.92-5.04 (m, 1H), 3.83 (dd, 2H), 3.43 (q, 2H), 3.34-3.38 (overlap. m, 2H), 3.13 (t, 2H), 2.55 (s, 3H), 2.12-2.26 (m, 2H), 1.80 (d, 2H), 1.06-1.18 (m, 6H).

### Example 39

### N-(3-Dimethylamino-2,2-dimethyl-propyl)-5-[[5-fluoro-4-[2-methyl-3-(oxan-4-yl)imidazol-4-yl]pyrimidin-2-yl]amino]pyridine-2-carboxamide

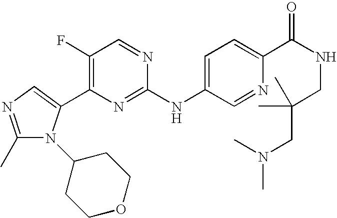

Amine: N,N,2,2-Tetramethylpropane-1,3-diamine

Yield: 51%

m/z* (M+1): 511

NMR: 10.04 (s, 1H), 8.88 (d, 1H), 8.76 (t, 1H), 8.66 (d, 1H), 8.16 (dd, 1H), 7.96 (d, 1H), 7.38 (d, 1H), 4.96-5.07 (m, 1H), 3.80 (dd, 2H), 3.22 (d, 2H), 3.08 (t, 2H), 2.55 (s, 3H), 2.26 (s, 6H), 2.15-2.21 (m, 4H), 1.81 (d, 2H), 0.88 (s, 6H).

### Example 40

### (3,5-Dimethyl-1-piperidyl)-[5-[[5-fluoro-4-[2-methyl-3-(oxan-4-yl)imidazol-4-yl]pyrimidin-2-yl]amino]pyridin-2-yl]methanone

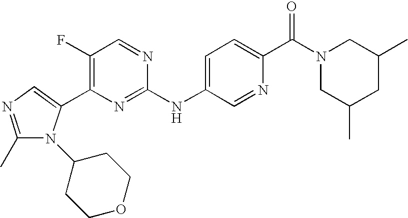

Amine: 3,5-Dimethylpiperidine

Yield: 30%

m/z* (M+1): 494

NMR: 9.93 (s, 1H), 8.78 (d, 1H), 8.64 (d, 1H), 8.12 (dd, 1H), 7.50 (d, 1H), 7.36 (d, 1H), 4.94-5.05 (m, 1H), 4.46 (d, 1H), 3.83-3.77 (m, 3H), 3.11 (q, 2H), 2.54 (s, 3H), 2.26-2.13 (m, 3H), 1.79 (d, 3H), 1.58 (br. s., 2H), 0.91 (d, 3H), 0.81 (q, 1H), 0.73 (d, 3H).
Purity analysis was run on a Water Acquity system with PDA (Waters 2996) and Waters ZQ mass spectrometer. Column; Acquity UPLC™ BEH C₈ 1.7 μm 2.1×50 mm. The column temperature was set to 65° C. A linear 2 min gradient from 100% A (A: 95% 0.01 M NH₄OAc in MilliQ water and 5% MeCN) to 100% B (5% 0.01 M N₄OAc in MilliQ water and 95% MeCN) was applied for LC-separation at flow rate 1.2 mL/min. The PDA was scanned from 210-350 nm and 254 nm was extracted for purity determination. The ZQ mass spectrometer was run with ESI in pos/neg switching mode. The Capillary Voltage was 3 kV and the Cone Voltage was 30V.

### Example 41

### Methyl 5-[[5-fluoro-4-[2-methyl-3-(oxan-4-yl)imidazol-4-yl]pyrimidin-2-yl]amino]pyridine-2-carboxylate

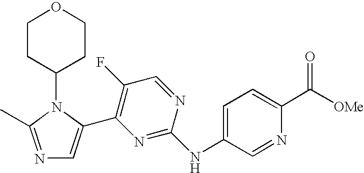

General Method A was followed using 5-fluoro-4-[2-methyl-3-(oxan-4-yl)imidazol-4-yl]pyrimidin-2-amine (as described in Example 6) (1.30 g, 4.69 mmol), methyl 5-bromopyridine-2-carboxylate (1.42 g, 6.56 mmol), Cs₂CO₃ (2.44 g, 7.50 mmol), Pd₂(dba)₃ (215 mg, 0.23 mmol) and X-Phos (224 mg, 0.47 mmol). The mixture was heated at 90° C. for 7 h and kept at r.t over night followed by the addition of methyl 5-bromopyridine-2-carboxylate (0.48 g, 2.22 mmol), Cs₂CO₃ (0.41 g, 1.26 mmol), Pd₂(dba)₃ (60 mg, 0.066 mmol), X-Phos (62 mg, 0.13 mmol) and 1,4-dioxane (5 mL). The mixture was heated at 90° C. for 4.5 h. Work-up by Method 1 and silica chromatography (0→7% MeOH in DCM) gave a yellow sticky solid. Trituration with CH₃CN and recrystallization from EtOH gave the title compound (1.3 g, 67%).

¹H NMR (400 MHz, CDCl₃) δ ppm 8.75 (d, 1H), 8.38 (d, 1H), 8.34 (dd, 1H), 8.12 (d, 1H), 7.70 (d, 1H), 7.47 (br. s, 1H), 5.10-5.03 (m, 1H), 4.10 (dd, 2H), 4.01 (s, 3H) 3.75-3.71 (m, 1H), 3.36-3.29 (m, 2H), 2.67 (s, 3H), 2.59-2.51 (m, 2H), 1.91-1.87 (m, 2H); MS (ESI) m/z 413 (M+1).

### Example 42

### Azetidin-1-yl-[3-chloro-5-[[5-fluoro-4-[2-methyl-3-(oxan-4-yl)imidazol-4-yl]pyrimidin-2-yl]amino]pyridin-2-yl]methanone

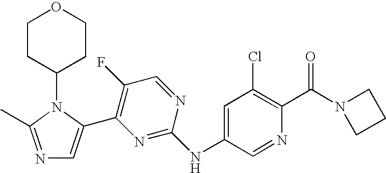

General Method A was followed using 5-fluoro-4-[2-methyl-3-(oxan-4-yl)imidazol-4-yl]pyrimidin-2-amine (as described in Example 6) (0.070 g, 0.252 mmol), azetidin-1-yl-(3,5-dichloropyridin-2-yl)methanone (as described in Example 48(a)) (0.0583 g, 0.252 mmol), Cs₂CO₃ (0.131 g, 0.403 mmol), Pd₂(dba)₃ (22.9 mg, 0.025 mmol) and X-Phos (23.8 mg, 0.050 mmol). The mixture was heated at 90° C. for 24 h and kept at r.t over night followed by the addition of Pd₂(dba)₃ (14 mg, 0.0153 mmol) and X-Phos (16 mg, 0.0336 mmol). The mixture was heated at 90° C. for 6 h. Work-up by Method 2 and preparative HPLC followed by silica chromatography (0→5% MeOH in DCM) gave the title compound (0.0145 g) in 6.6% yield.

¹H NMR (400 MHz, CDCl₃) δ ppm 8.49 (d, 1H), 8.36-8.33 (m, 2H), 7.93 (br. s, 1H), 7.66 (d, 1H), 5.13-5.05 (m, 1H), 4.27-4.23 (m, 2H), 4.19-4.16 (m, 2H), 4.14-4.07 (m, 2H), 3.38-3.32 (m, 2H), 2.64 (s, 3H), 2.55-2.45 (m, 2H), 2.37-2.29 (m, 2H), 1.91-1.87 (m, 2H); MS (ESI) m/z 471 (M−1).

### Example 43

### [3-Chloro-5-[[5-fluoro-4-[3-(oxan-4-yl)-2-(trifluoromethyl)imidazol-4-yl]pyrimidin-2-yl]amino]pyridin-2-yl]-(4-methylpiperazin-1-yl)methanone

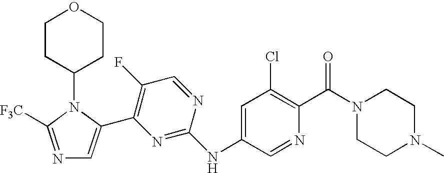

General Method A using 5-fluoro-4-[3-(oxan-4-yl)-2-(trifluoromethyl)imidazol-4-yl]pyrimidin-2-amine (as described in Example 8) (0.060 g, 0.181 mmol), (3,5-dichloropyridin-2-yl)-(4-methylpiperazin-1-yl)methanone (as described in Example 49(a)) (0.0496 g, 0.181 mmol), Cs₂CO₃ (0.094 g, 0.29 mmol), Pd₂(dba)₃ (16.5 mg, 0.018 mmol) and X-Phos (17.2 mg, 0.036 mmol). The mixture was heated at 90° C. for 17 h, followed by the addition of Pd₂(dba)₃ (12 mg, 0.013 mmol) and X-Phos (13 mg, 0.027 mmol) and then was heated at 90° C. for an additional 3 h. Work-up by Method 2 and purification by preparative HPLC gave the title compound (0.033 g) in 15% yield.

¹H NMR (400 MHz, CDCl₃) δ ppm 8.52 (d, 1H), 8.47 (d, 1H), 8.36 (d, 1H), 8.10 (br. s, 1H), 7.73 (d, 1H), 4.89-4.81 (m, 1H), 4.12 (dd, 2H), 3.86-3.83 (m, 2H), 3.51-3.44 (m, 2H), 3.29-3.27 (m, 2H), 2.76-2.66 (m, 2H), 2.52-2.50 (m, 2H), 2.40-2.37 (m, 2H), 2.32 (s, 3H), 1.90-1.86 (m, 2H); MS (ESI) m/z 570 (M+1).

### Example 44

### [3-Chloro-5-[[5-fluoro-4-[3-methyl-2-(trifluoromethyl)imidazol-4-yl]pyrimidin-2-yl]amino]pyridin-2-yl]-(4-methylpiperazin-1-yl)methanone

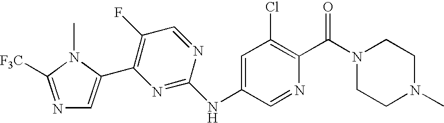

General Method A was followed using 5-fluoro-4-[3-methyl-2-(trifluoromethyl)imidazol-4-yl]pyrimidin-2-amine (as described in Example 9) (0.16 g, 0.62 mmol), (3,5-dichloropyridin-2-yl)-(4-methylpiperazin-1-yl)methanone (as described in Example 49(a)) (0.17 g, 0.62 mmol), Cs₂CO₃ (0.32 g, 0.99 mmol), Pd₂(dba)₃ (43.0 mg, 0.047 mmol) and X-Phos (44.3 mg, 0.093 mmol). The mixture was heated at 90° C. for 17 h. Work-up by Method 1 and purification by preparative HPLC gave the title compound (0.054 g) in 17% yield.

¹H NMR (400 MHz, DMSO-d₆) δ ppm 10.35 (s, 1H), 8.82 (d, 1H), 8.75 (d, 1H), 8.45 (d, 1H), 7.77 (d, 1H), 4.11 (s, 3H), 3.61-3.67 (m, 2H), 3.10-3.16 (m, 2H), 2.32-2.41 (m, 2H), 2.21-2.30 (m, 2H), 2.19 (s, 3H); MS (ESI) m/z 498 (M−1).

### Example 45

### Azetidin-1-yl-[3-chloro-5-[[5-fluoro-4-[3-methyl-2-(trifluoromethyl)imidazol-4-yl]pyrimidin-2-yl]amino]pyridin-2-yl]methanone hydrochloride

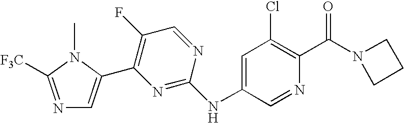

General Method A was followed using 5-fluoro-4-[3-methyl-2-(trifluoromethyl)imidazol-4-yl]pyrimidin-2-amine (as described in Example 9) (0.20 g, 0.75 mmol), azetidin-1-yl-(3,5-dichloropyridin-2-yl)methanone (as described in Example 48(a)) (0.17 g, 0.75 mmol), Cs₂CO₃ (0.39 g, 1.2 mmol), Pd₂(dba)₃ (51.7 mg, 0.056 mmol) and X-Phos (53.8 mg, 0.11 mmol). The mixture was heated at 90° C. for 17 h. Work-up by Method 1 and purification by preparative HPLC followed by hydrochloride formation gave the title compound (0.054 g) in 15% yield.

¹H NMR (400 MHz, DMSO-d₆) δ ppm 10.41 (s, 1H), 8.83 (d, 1H), 8.75 (d, 1H), 8.45 (d, 1H), 7.77 (d, 1H), 4.11 (s, 3H), 4.01-4.08 (m, 4H), 2.20-2.31 (m, 2H); MS (ESI) m/z 457 (M+1).

### Example 46

### N-[6-(Azetidin-1-ylcarbonyl)pyridin-3-yl]-4-(1,2-dimethyl-1H-imidazol-5-yl)-5-fluoropyrimidin-2-amine

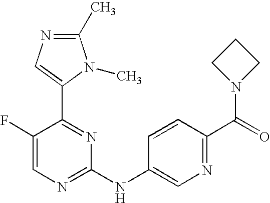

The title compound was prepared in accordance with the general method A using 4-(1,2-dimethyl-1H-imidazol-5-yl)-5-fluoropyrimidin-2-amine (as described in Example 7) (30 mg, 0.145 mmol) and 2-(azetidin-1-ylcarbonyl)-5-bromopyridine (41 mg, 0.17 mmol) (reported in WO 2005014571) to give the title compound (22 mg, 41%).

¹H NMR (400 MHz, CDCl₃) δ ppm 8.69 (d, J=2.53 Hz, 1H) 8.31 (d, J=3.03 Hz, 1H) 8.14-8.20 (m, 1H) 8.07-8.12 (m, 1H) 7.77 (d, J=4.29 Hz, 1H) 7.36-7.50 (m, 1H) 4.72 (t, J=7.71 Hz, 2H) 4.25 (t, 2H) 3.93 (s, 3H) 2.50 (s, 3H) 2.31-2.44 (m, 3H); MS (ESI) m/z 368 (M+1).

### Example 47

### 4-(1,2-Dimethyl-1H-imidazol-5-yl)-5-fluoro-N-{6-[(4-methylpiperazin-1-yl)carbonyl]pyridin-3-yl}pyrimidin-2-amine

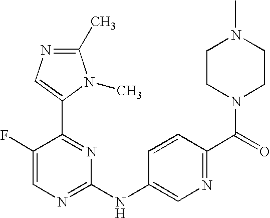

The title compound was prepared in accordance with the general method A using 4-(1,2-dimethyl-1H-imidazol-5-yl)-5-fluoropyrimidin-2-amine (as described in Example 7) (40 mg, 0.193 mmol) and 1-[(5-bromopyridin-2-yl)carbonyl]-4-methylpiperazine (as described in Example 4(b)) (55 mg, 0.23 mmol) to give the title compound (45 mg, 57%).

¹H NMR (400 MHz, CDCl₃) δ ppm 8.70 (d, J=2.53 Hz, 1H) 8.30 (d, J=3.28 Hz, 1H) 8.18 (dd, J=8.59, 2.53 Hz, 1H) 7.76 (d, J=4.29 Hz, 1H) 7.70 (d, J=8.59 Hz, 1H) 3.93 (s, 3H) 3.81-3.87 (m, J=5.81 Hz, 2H) 3.71-3.78 (m, 2H) 2.50-2.57 (m, 2H) 2.49 (s, 3H) 2.40-2.46 (m, 2H) 2.33 (s, 3H); MS (ESI) m/z 411 (M+1).

### Example 48

### N-[6-(Azetidin-1-ylcarbonyl)-5-chloropyridin-3-yl]-4-(1,2-dimethyl-1H-imidazol-5-yl)-5-fluoropyrimidin-2-amine

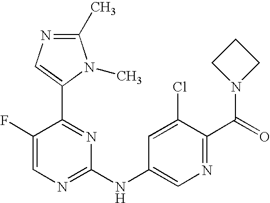

### Example 48(a)

### 2-(Azetidin-1-ylcarbonyl)-3,5-dichloropyridine

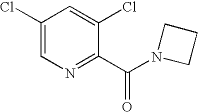

The title compound was prepared in accordance with the general method C using 3,5-dichloropyridine-2-carboxylic acid (500 mg, 2.6 mmol) and azetidine (150 mg, 2.6 mmol) to give the title compound (430 mg, 72%).

¹H NMR (400 MHz, CDCl₃) δ ppm 8.45 (d, J=2.02 Hz, 1H) 7.80 (d, J=2.02 Hz, 1H) 4.27 (t, J=7.83 Hz, 2H) 4.15 (t, J=7.71 Hz, 2H) 2.28-2.42 (m, 2H); MS (ESI) m/z 231 (M+1).

### Example 48(b)

### N-[6-(Azetidin-1-ylcarbonyl)-5-chloropyridin-3-yl]-4-(1,2-dimethyl-1H-imidazol-5-yl)-5-fluoropyrimidin-2-amine

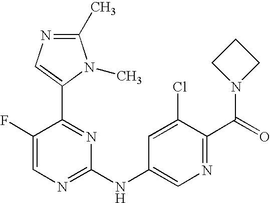

The title compound was prepared in accordance with the general method A using 4-(1,2-dimethyl-1H-imidazol-5-yl)-5-fluoropyrimidin-2-amine (as described in Example 7) (50 mg, 0.24 mmol) and 2-(azetidin-1-ylcarbonyl)-3,5-dichloropyridine (as described above) (57 mg, 0.25 mmol) to give the title compound (26 mg, 27%).

¹H NMR (400 MHz, CDCl₃) δ ppm 8.50 (d, J=2.27 Hz, 1H) 8.39 (d, J=2.27 Hz, 1H) 8.30 (d, J=3.28 Hz, 1H) 8.07 (s, 1H) 7.77 (d, J=4.29 Hz, 1H) 4.26 (t, J=7.83 Hz, 2H) 4.19 (t, 2H) 3.94 (s, 3H) 2.49 (s, 3H) 2.29-2.39 (m, 2H); MS (ESI) m/z 402 (M+1).

### Example 49

### N-[5-Chloro-6-[(4-methylpiperazin-1-yl)carbonyl]pyridin-3-yl]-4-(1,2-dimethyl-1H-imidazol-5-yl)-5-fluoropyrimidin-2-amine

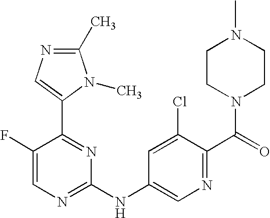

### Example 49(a)

### (3,5-Dichloropyridin-2-yl)-(4-methylpiperazin-1-yl)methanone

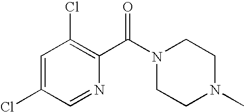

The title compound was prepared in accordance with the general method C using 3,5-dichloropyridine-2-carboxylic acid (555 mg, 2.89 mmol) and 1-methylpiperazine (320 μL, 2.89 mmol) to give the title compound (417 mg, 53%).

¹H NMR (400 MHz, CDCl₃) δ ppm 8.49 (d, J=2.02 Hz, 1H) 7.79 (d, J=2.02 Hz, 1H) 3.82-3.88 (m, 2H) 3.22-3.27 (m, 2H) 2.50-2.55 (m, 2H) 2.37-2.42 (m, 2H) 2.33 (s, 3H); MS (ESI) m/z 274 (M+1).

### Example 49(b)

### N-{5-Chloro-6-[(4-methylpiperazin-1-yl)carbonyl]pyridin-3-yl}-4-(1,2-dimethyl-1H-imidazol-5-yl)-5-fluoropyrimidin-2-amine

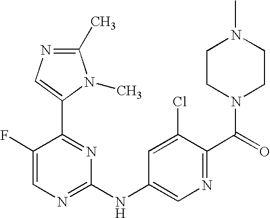

The title compound was prepared in accordance with the general method A using 4-(1,2-dimethyl-1H-imidazol-5-yl)-5-fluoropyrimidin-2-amine (as described in Example 7) (50 mg, 0.24 mmol) and (3,5-dichloropyridin-2-yl)-(4-methylpiperazin-1-yl)methanone (as described above) (66 mg, 0.24 mmol) to give the title compound (29 mg, 27%).

¹H NMR (400 MHz, CDCl₃) δ ppm 8.47 (d, J=2.27 Hz, 1H) 8.36 (d, J=2.27 Hz, 1H) 8.28 (d, J=3.03 Hz, 1H) 8.18 (s, 1H) 7.76 (d, J=4.29 Hz, 1H) 3.94 (s, 3H) 3.85 (t, 2H) 3.29 (t, 2H) 2.51 (t, 2H) 2.48 (s, 3H) 2.39 (t, 2H) 2.32 (s, 3H); MS (ESI) m/z 445 (M+1).

### Example 50

### {5-Fluoro-4-[2-methyl-3-(tetrahydro-pyran-4-yl)-3H-imidazol-4-yl]-pyrimidin-2-yl}-[6-(propan-2-ylsulfonyl)-pyridin-3-yl]-amine

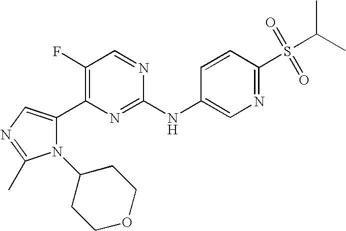

### Example 50(a)

### 5-Bromo-2-isopropylsulfanyl-pyridine

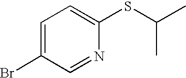

5-Bromo-2-chloro-pyridine (516.0 mg, 2.681 mmol) was dissolved in DMF (10 mL) and sodium 2-propanethiolate (1.5 g, 15.28 mmol) was added at room temperature. The reaction mixture was stirred at room temperature for 1 hour where after analysis by GC-MS showed only little starting material left. Water (10 mL) was added followed by extraction with CH₂Cl₂ (3×20 mL). The combined organic phases were dried (MgSO₄), filtered and concentrated to afford the title compound (600 mg, 96%).

¹H NMR (400 MHz, CDCl₃) δ ppm 1.38 (d, J=6.82 Hz, 6H) 3.87-4.02 (m, 1H) 7.04 (d, J=8.59 Hz, 1H) 7.56 (dd, J=8.59, 2.53 Hz, 1H) 8.47 (d, J=1.77 Hz, 1H); MS (ESI) m/z 233 (M+1).

### Example 50(b)

### 5-Bromo-2-(propan-2-ylsulfonyl)-pyridine

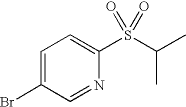

5-Bromo-2-isopropylsulfanyl-pyridine (271.8 mg, 1.171 mmol) was dissolved in CH₂Cl₂ (2.5 mL) and mCPBA (1010 mg, 2.927 mmol) was added portionwise. The reaction mixture was stirred at room temperature for 30 min where after analysis by LC-MS showed that all starting material was transformed into desired product. The reaction was quenched by addition of NaOH (5 mL, 1M) and another 5 mL of CH₂Cl₂ was added followed by extraction with CH₂Cl₂ (3×10 mL), and washing with water (10 mL). The combined organic phases were dried (MgSO₄), filtered and concentrated to afford the title compound (266 mg, 86%).

¹H NMR (400 MHz, CDCl₃) δ ppm 1.34 (d, J=6.82 Hz, 6H) 3.68-3.80 (m, 1H) 7.99 (d, J=7.58 Hz, 1H) 8.11 (dd, J=8.21, 2.15 Hz, 1H) 8.80-8.84 (m, 1H); MS (ESI) m/z 265 (M+1).

### Example 50(c)

### {5-Fluoro-4-[2-methyl-3-(tetrahydro-pyran-4-yl)-3H-imidazol-4-yl]-pyrimidin-2-yl}-[6-(propan-2-ylsulfonyl)-pyridin-3-yl]-amine

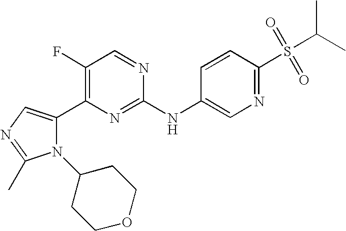

5-Fluoro-4-[2-methyl-1-(tetrahydro-2H-pyran-4-yl)-1H-imidazol-5-yl]pyrimidin-2-amine (as described in Example 6) (157.3 mg, 0.567 mmol), 5-bromo-2-(propan-2-ylsulfonyl)-pyridine (149.8 mg, 0.567 mmol), Cs₂CO₃ (370 mg, 1.134 mmol), Pd₂(dba)₃ (26 mg, 0.028 mmol) and XantPhos (33 mg, 0.057 mmol) were weighed out in a 50 mL round-bottom flask and dioxane (13 mL) was added. The system was flushed with argon and then heated to 90° C. and stirred for 17 hours. Water (60 mL) was added and the mixture was extracted with CH₂Cl₂ (3×60 mL). Drying (Na₂SO₄), filtration and concentration afforded a crude material, which was purified by preparative HPLC to afford the title compound (102 mg, 39%).

¹H NMR (400 MHz, CDCl₃) δ ppm 1.31 (d, J=6.82 Hz, 6H) 1.87 (dd, J=13.14, 3.79 Hz, 2H) 2.45-2.59 (m, 2H) 2.64 (s, 3H) 3.31-3.41 (m, 2H) 3.59-3.71 (m, 1H) 4.09 (dd, J=11.62, 4.55 Hz, 2H) 4.99-5.10 (m, 1H) 7.67 (d, J=4.04 Hz, 1H) 7.98 (d, J=8.84 Hz, 1H) 8.26 (s, 1H) 8.36-8.41 (m, 2H) 8.86 (d, J=2.02 Hz, 1H); MS (ESI) m/z 462 (M+1).

### Example 51

### (6-Ethanesulfonyl-pyridin-3-yl)-{5-fluoro-4-[2-methyl-3-(tetrahydro-pyran-4-yl)-3H-imidazol-4-yl]-pyrimidin-2-yl}-amine

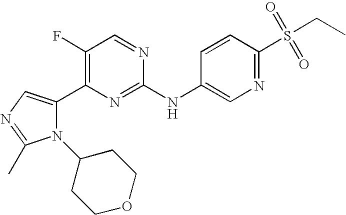

### Example 51(a)

### 5-Bromo-2-ethylsulfanyl-pyridine

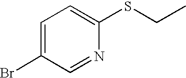

5-Bromo-2-chloro-pyridine (5.0 g, 25.98 mmol) was dissolved in DMF (94 mL) and sodiumethanethiolate (10.9 g, 129.9 mmol) was added at room temperature. The reaction mixture was stirred at room temperature for 1 hour where after analysis by GC-MS showed only small amounts of starting material left. Water (100 mL) was added followed by extraction with CH₂Cl₂ (3×200 mL). The combined organic phases were dried (MgSO₄), filtered and concentrated to afford the title compound (5.0 g, 88%).

¹H NMR (400 MHz, CDCl₃) δ ppm 1.32-1.42 (m, 3H) 3.09-3.20 (m, 2H) 7.05 (dd, J=8.46, 2.65 Hz, 1H) 7.53-7.61 (m, 1H) 8.47 (s, 1H); MS (ESI) m/z 219 (M+1).

### Example 51(b)

### 5-Bromo-2-ethanesulfonyl-pyridine

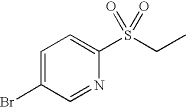

5-Bromo-2-ethylsulfanyl-pyridine (5.0 g, 22.92 mmol) was dissolved in CH₂Cl₂ (62 mL) and mCPBA (12.9 g, 57.3 mmol) was added portionwise. The reaction mixture was stirred at room temperature for 30 min where after analysis by LC-MS showed that all starting material was transformed into desired product. The reaction was quenched by addition of NaOH (100 mL, 1M) and another 100 mL of CH₂Cl₂ was added followed by extraction with CH₂Cl₂ (3×200 mL), and washing with water (200 mL). The combined organic phases were dried (MgSO₄), filtered and concentrated to afford the title compound (5.7 g, 99%).

¹H NMR (400 MHz, CDCl₃) δ ppm 1.30 (t, 3H) 3.41 (q, J=7.58 Hz, 2H) 7.99 (d, J=7.58 Hz, 1H) 8.11 (dd, J=8.34, 2.27 Hz, 1H) 8.80 (d, J=2.27 Hz, 1H); MS (ESI) m/z 251 (M+1).

### Example 51(c)

### (6-Ethanesulfonyl-pyridin-3-yl)-{5-fluoro-4-[2-methyl-3-(tetrahydro-pyran-4-yl)-3H-imidazol-4-yl]-pyrimidin-2-yl}-amine

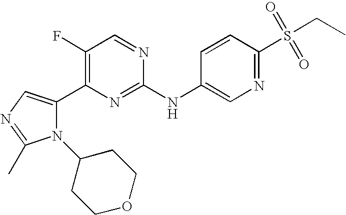

5-Fluoro-4-[2-methyl-1-(tetrahydro-2H-pyran-4-yl)-1H-imidazol-5-yl]pyrimidin-2-amine (as described in Example 6) (836.6 mg, 3.017 mmol), 5-bromo-2-ethanesulfonyl-pyridine (754.6 mg, 3.017 mmol), Cs₂CO₃ (2.0 g, 6.033 mmol), Pd₂(dba)₃ (138 mg, 0.151 mmol) and XantPhos (175 mg, 0.302 mmol) were weighed out in a 250 mL round-bottom flask and dioxane (68 mL) was added. The system was flushed with argon and then heated to 90° C. and stirred for 17 hours. Water (150 mL) was added and the mixture was extracted with CH₂Cl₂ (3×150 mL). The combined organic phases were washed with HCl (4×100 mL, 2M). The combined acidic H₂O-phases were neutralized with 50% NaOH (aq) until neutral or slightly basic and then extracted with CH₂Cl₂ (3×150 mL). Drying (Na₂SO₄), filtration and concentration afforded a crude material, which was purified by preparative HPLC to afford the title compound (790 mg, 59%).

¹H NMR (400 MHz, CDCl₃) δ ppm 1.30 (t, J=7.45 Hz, 3H) 1.89 (dd, J=12.88, 4.04 Hz, 2H) 2.52-2.65 (m, 2H) 2.67 (s, 3H) 3.34-3.44 (m, 4H) 4.13 (dd, J=11.62, 4.80 Hz, 2H) 4.97-5.10 (m, 1H) 7.71 (d, J=3.79 Hz, 1H) 7.87 (s, 1H) 8.02 (d, J=8.59 Hz, 1H) 8.37-8.42 (m, 2H) 8.85 (d, J=2.53 Hz, 1H); MS (ESI) m/z 448 (M+1).

### Example 52

### 5-[[5-Fluoro-4-[2-methyl-3-(oxan-4-yl)-2,4-dihydroimidazol-4-yl]pyrimidin-2-yl]amino]-N-(2,2,2-trifluoroethyl)pyridine-2-sulfonamide

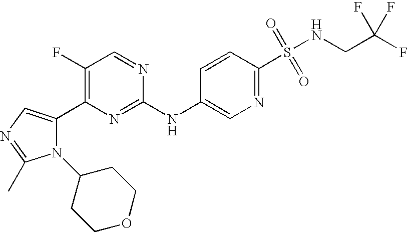

### Example 52(a)

### 2-Benzylsulfanyl-5-bromo-pyridine

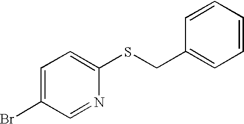

Potassium tert-butoxide (2.79 g, 24.84 mmol) was dissolved in DMF (10 mL) and benzylmercaptane (2.57 g, 20.70 mmol) was added dropwise at 0° C. The mixture was stirred at room temperature for 15 min and then cooled to 0° C. 5-Bromo-2-chloro-pyridine (3.98 g, 20.70 mmol) dissolved in DMF (4 mL) was added dropwise at 0° C. and the mixture was heated at 80° C. for 1.5 hours. The mixture was poured into water (100 mL) and extracted with ether (3×100 mL). The combined organic phases were washed with brine (100 mL), water (100 mL) and dried (Na₂SO₄). Concentration afforded the title compound (5.52 g, 95%). MS (ESI) m/z 281 (M+1).

### Example 52(b)

### 5-Bromopyridine-2-sulfonyl chloride

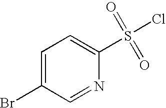

2-Benzylsulfanyl-5-bromo-pyridine (3.0 g, 10.71 mmol) was dissolved in CH₂Cl₂ (500 mL) in a 1 L Schlenk flask and water (250 mL) and HCOOH (250 mL) was added. The heterogeneous mixture was cooled to 0° C. and Cl₂ gas was bubbled through the vigorously stirred mixture from a Pasteur pipette. The chlorine gas addition was continued for three minutes or until the mixture turned deep yellow. The organic phase was separated and diluted with CH₂Cl₂ (100 mL). The aqueous phase was extracted with CH₂Cl₂ (3×250 mL) and the combined organic phases were washed with 1M NaOH (500 mL) followed by brine (500 mL). Drying (Na₂SO₄) and concentration afforded the title compound (2.73 g, 99%). MS (ESI) m/z 258 (M+1).

### Example 52(c)

### 5-Bromo-pyridine-2-sulfonic acid (2,2,2-trifluoro-ethyl)-amide

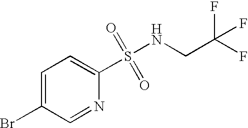

5-Bromopyridine-2-sulfonyl chloride (100.7 mg, 0.393 mmol) was dissolved in CH₂Cl₂ (1 mL) and 2,2,2-trifluoro-ethylamine (34 μL, 0.432 mmol) was added. Stirring was continued at room temperature for 3 hours and saturated aqueous NaHCO₃ (1 mL) was added. The mixture was diluted with CH₂Cl₂ (5 mL) and the aqueous phase was extracted with CH₂Cl₂ (3×5 mL). The combined organic phases were dried (Na₂SO₄) and concentrated to afford the title compound (49 mg, 39%). MS (ESI) m/z 320 (M+1).

### Example 52(d)

### 5-[[5-Fluoro-4-[2-methyl-3-(oxan-4-yl)-2,4-dihydroimidazol-4-yl]pyrimidin-2-yl]amino]-N-(2,2,2-trifluoroethyl)pyridine-2-sulfonamide

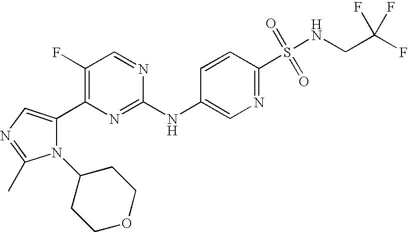

5-Fluoro-4-[2-methyl-1-(tetrahydro-2H-pyran-4-yl)-1H-imidazol-5-yl]pyrimidin-2-amine (as described in Example 6) (42.1 mg, 0.152 mmol), 5-bromo-pyridine-2-sulfonic acid (2,2,2-trifluoro-ethyl)-amide (48.5 mg, 0.152 mmol), Cs₂CO₃ (79.2 mg, 0.243 mmol), Pd₂(dba)₃ (7 mg, 0.008 mmol) and XantPhos (9 mg, 0.016) mmol) were weighed out in a 25 mL round-bottom flask and dioxane (3 mL) was added. The system was flushed with argon and then heated to 90° C. and stirred for 17 hours. Water (30 mL) was added and the mixture was extracted with CH₂Cl₂ (3×30 mL). Drying (Na₂SO₄), filtration and concentration afforded a crude material, which was purified by preparative HPLC to afford the title compound (3 mg, 3%).

¹H NMR (400 MHz, CDCl₃) δ ppm 1.83-1.92 (m, 2H) 2.53-2.65 (m, 2H) 2.68 (s, 3H) 3.39 (t, J=11.87 Hz, 2H) 3.73-3.85 (m, 2H) 4.11-4.20 (m, 2H) 5.00 (br. s., 1H) 5.74 (br. s., 1H) 7.73 (br. s., 1H) 7.81 (s, 1H) 7.93 (d, J=8.84 Hz, 1H) 8.33-8.42 (m, 2H) 8.81 (s, 1H); MS (ESI) m/z 516 (M+1).

### Example 53

### N,N-Dimethyl-5-[[4-[2-methyl-3-(oxan-4-yl)-2,4-dihydroimidazol-4-yl]pyrimidin-2-yl]amino]pyridine-2-sulfonamide

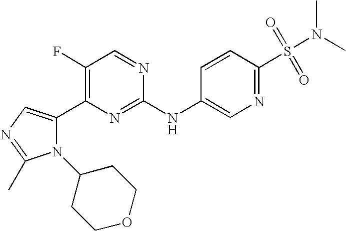

### Example 53(a)

### 5-Bromo-pyridine-2-sulfonic acid dimethylamide

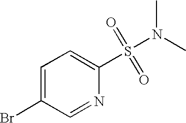

5-Bromopyridine-2-sulfonyl chloride (as described in Example 52) (129.4 mg, 0.505 mmol) was dissolved in CH₂Cl₂ (1 mL) and dimethylamine (29 μL, 0.555 mmol) was added. Stirring was continued at room temperature for 3 hours and saturated aqueous NaHCO₃ (1 mL) was added. The mixture was diluted with CH₂Cl₂ (5 mL) and the aqueous phase was extracted with CH₂Cl₂ (3×5 mL). The combined organic phases were dried (Na₂SO₄) and concentrated to afford the title compound (97 mg, 67%).

MS (ESI) m/z 266 (M+1).

### Example 53(b)

### N,N-Dimethyl-5-[[4-[2-methyl-3-(oxan-4-yl)-2,4-dihydroimidazol-4-yl]pyrimidin-2-yl]amino]pyridine-2-sulfonamide

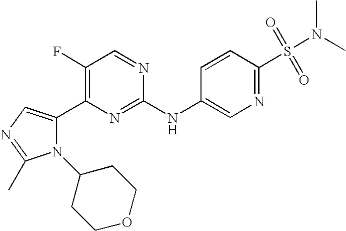

5-Fluoro-4-[2-methyl-1-(tetrahydro-2H-pyran-4-yl)-1H-imidazol-5-yl]pyrimidin-2-amine (as described in Example 6) (98.1 mg, 0.354 mmol), 5-bromo-pyridine-2-sulfonic acid dimethylamide (93.8 mg, 0.354 mmol), Cs₂CO₃ (230.5 mg, 0.708 mmol), Pd₂(dba)₃ (16 mg, 0.018 mmol) and XantPhos (21 mg, 0.035) mmol) were weighed out in a 25 mL round-bottom flask and dioxane (5 mL) was added. The system was flushed with argon and then heated to 90° C. and stirred for 17 hours. Water (30 mL) was added and the mixture was extracted with CH₂Cl₂ (3×30 mL). Drying (Na₂SO₄), filtration and concentration afforded a crude material which was purified by preparative HPLC to afford the title compound (9 mg, 6%).

¹H NMR (400 MHz, CDCl₃) δ ppm 1.88 (dd, J=12.63, 3.79 Hz, 2H) 2.50-2.63 (m, 2H) 2.66 (s, 3H) 2.91 (s, 6H) 3.31-3.42 (m, 2H) 4.12 (dd, J=11.62, 4.80 Hz, 2H) 4.98-5.09 (m, 1H) 7.70 (d, J=3.54 Hz, 1H) 7.86-7.91 (m, 2H) 8.32 (dd, J=8.72, 2.65 Hz, 1H) 8.38 (d, J=2.78 Hz, 1H) 8.83 (d, J=2.53 Hz, 1H); MS (ESI) m/z 462 (M+1).

### Example 54

### N-[5-Chloro-6-(piperidin-1-ylcarbonyl)pyridin-3-yl]-5-fluoro-4-[2-methyl-1-(tetrahydro-2H-pyran-4-yl)-1H-imidazol-5-yl]pyrimidin-2-amine hydrocholoride

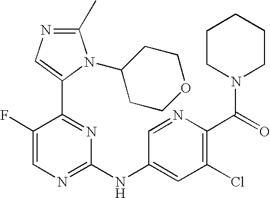

### Example 54(a)

### 3,5-Dichloro-2-(piperidin-1-ylcarbonyl)pyridine

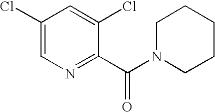

3,5-Dichloro-2-pyridine carboxylic acid (1.25 g, 6.5 mmol) was suspended in thionyl chloride (10 ml). DMF (2 drops) was added and the mixture was refluxed for 15 minutes under an atmosphere of nitrogen. The solvent was evaporated. Toluene was added and the solvent was evaporated to give a solid. The solid was dissolved in DCM (8 ml) and the mixture was cooled to 0° C. Piperidine (0.64 ml, 6.5 mmol) was added dropwise followed by triethylamine (0.91 ml, 6.5 mmol). The cooling bath was removed. The mixture was stirred under nitrogen atmosphere until RT was reached and then for an additional 15 minutes. The mixture was washed with aqueous sodium bicarbonate and the organic phase was dried (MgSO₄), filtered and concentrated. The residue was purified by column chromatography eluting with gradients of heptane and ethyl acetate to give the title compound (1.28 g, 76%).

¹H NMR (400 MHz, CDCl₃) δ ppm 8.46 (d, 1H) 7.77 (d, 1H) 3.74 (m., 2H) 3.11-3.16 (m, 2H) 1.64-1.71 (m, 4H) 1.55 (m, 2H) MS (ESI) m/z 259; 261 (M+1).

### Example 54(b)

### N-[5-Chloro-6-(piperidin-1-ylcarbonyl)pyridin-3-yl]-5-fluoro-4-[2-methyl-1-(tetrahydro-2H-pyran-4-yl)-1H-imidazol-5-yl]pyrimidin-2-amine hydrochloride

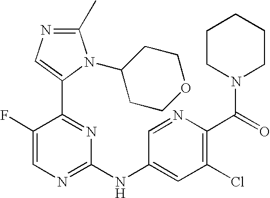

5-Fluoro-4-[2-methyl-1-(tetrahydro-2H-pyran-4-yl)-1H-imidazol-5-yl]pyrimidin-2-amine (as described in Example 6) (150 mg, 0.54 mmol), 3,5-dichloro-2-(piperidin-1-ylcarbonyl)pyridine (as described in Example 54(a)) (124 mg, 0.48 mmol) and caesium carbonate (351 mg, 1.08 mmol) were suspended in dioxane (4 ml). Pd₂(dba)₃ (26 mg, 0.029 mmol) and Xantphos (27 mg, 0.047 mmol) were added and the mixture was stirred at 90° C. under argon atmosphere for 16 h. Pd₂(dba)₃ (5 mg) was added and the mixture was heated at 90° C. for 4 h. The mixture was diluted with DCM and filtered through diatomeous earth. The organic phase was washed with aqueous sodium bicarbonate and evaporated. The residue was dissolved in DMSO and purified by preparative HPLC. The fractions containing the product were pooled and concentrated. Aqueous sodium bicarbonate was added and the mixture was extracted with DCM (×4). The organic phase was dried (MgSO₄), filtered and concentrated. The residue was dissolved in DCM (1 ml) and 1 M hydrochloric acid in ether (0.2 ml) was added. The solvent was evaporated to give the title compound (40 mg, 14%).

¹H NMR (400 MHz, DMSO-d₆) δ ppm 7.39-7.43 (m, 2H) 7.14 (d, 1H) 6.67 (d, 1H) 3.88-3.99 (m, 1H) 2.74 (dd, 2H) 1.95-2.07 (m, 4H) 1.64 (s, 3H) 1.06-1.20 (m, 2H) 0.80 (dd, 2H) 0.44-0.52 (m, 4H) 0.31-0.40 (m, 2H). MS (ESI) m/z 500; 502 (M+1).

### Example 55

### N-[5-Chloro-6-(piperidin-1-ylcarbonyl)pyridin-3-yl]-4-(1,2-dimethyl-1H-imidazol-5-yl)-5-fluoropyrimidin-2-amine hydrochloride

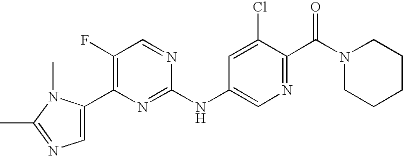

(1,2-Dimethyl-1H-imidazol-5-yl)-5-fluoropyrimidin-2-amine (as described in Example 7) (50 mg, 0.24 mmol), 3,5-dichloro-2-(piperidin-1-ylcarbonyl)pyridine (as described in Example 54(a)) (62 mg, 0.24 mmol) and caesium carbonate (156 mg, 0.48 mmol) were suspended in dioxane (2 ml). Pd₂(dba)₃ (22 mg, 0.024 mmol) and Xantphos (23 mg, 0.040 mmol) were added and the mixture was stirred at 90° C. under argon atmosphere for 16 h. The mixture was diluted with DCM and filtered through diatomeous earth. The organic phase was washed with aqueous sodium bicarbonate and concentrated. The residue was dissolved in DMSO and purified by preparative HPLC. The fractions containing the product were pooled and concentrated. Aqueous sodium bicarbonate was added and the mixture was extracted with DCM (×4). The organic phase was dried (MgSO₄), filtered and concentrated. The residue was dissolved in DCM (1 ml) and 1 M hydrochloric acid in ether (0.1 ml) was added. The solvent was evaporated to give the title compound (28 mg, 25%).

¹H NMR (400 MHz, DMSO-d₆) δ ppm 10.40 (s, 1H) 8.85 (d, 1H) 8.78 (d, 1H) 8.41 (d, 1H) 8.21 (d, 1H) 4.03 (s, 3H) 3.59-3.65 (m, 2H) 3.07-3.12 (m, 2H) 2.68 (s, 3H) 1.42-1.66 (m, 6H). MS (ESI) m/z 430; 432 (M+1).

### Example 56

### N-[5-Chloro-6-(piperidin-1-ylcarbonyl)pyridin-3-yl]-5-fluoro-4-[1-methyl-2-(trifluoromethyl)-1H-imidazol-5-yl]pyrimidin-2-amine hydrochloride

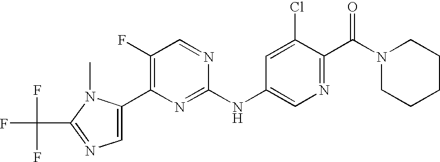

5-Fluoro-4-[1-methyl-2-(trifluoromethyl)-1H-imidazol-5-yl]pyrimidin-2-amine (as described in Example 9) (130 mg, 0.50 mmol), 3,5-dichloro-2-(piperidin-1-ylcarbonyl)pyridine (as described in Example 54(a)) (130 mg, 0.50 mmol) and caesium carbonate (326 mg, 1.0 mmol) were suspended in dioxane (4 ml). Pd₂(dba)₃ (27 mg, 0.030 mmol) and Xantphos (29 mg, 0.050 mmol) were added and the mixture was stirred at 90° C. under argon atmosphere for 16 h. Pd₂(dba)₃ (10 mg, 0.011 mmol) was added and the mixture was heated at 90° C. under argon atmosphere for 6 h. The mixture was diluted with DCM and filtered through diatomeous earth. The organic phase was washed with aqueous sodium bicarbonate and concentrated. The residue was dissolved in DMSO and purified by preparative HPLC. The fractions containing the product were pooled and concentrated. Aqueous sodium bicarbonate was added and the mixture was extracted with DCM (×4). The organic phase was dried (MgSO₄), filtered and concentrated. The residue was dissolved in DCM (1 ml) and 1 M hydrochloric acid in ether (0.1 ml) was added. The solvent was evaporated to give the title compound (13 mg, 5%).

¹H NMR (400 MHz, DMSO-d₆) δ ppm 10.34 (s, 1H) 8.81 (d, 1H) 8.75 (d, 1H) 8.43 (d, 1H) 7.76 (d, 1H) 4.10 (s, 3H) 3.58-3.65 (m, 2H) 3.05-3.13 (m, 2H) 1.42-1.66 (m, 6 H). MS (ESI) m/z 482; 484 (M+1).

### Example 57

### N-[5-Chloro-6-(piperidin-1-ylcarbonyl)pyridin-3-yl]-5-fluoro-4-[1-(tetrahydro-2H-pyran-4-yl)-2-(trifluoromethyl)-1H-imidazol-5-yl]pyrimidin-2-amine hydrochloride

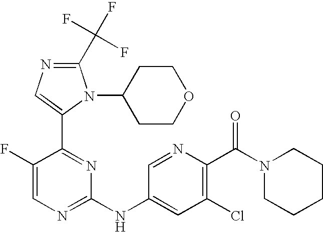

5-Fluoro-4-[1-(tetrahydro-2H-pyran-4-yl)-2-(trifluoromethyl)-1H-imidazol-5-yl]pyrimidin-2-amine (as described in Example 8) (70 mg, 0.21 mmol), 3,5-dichloro-2-(piperidin-1-ylcarbonyl)pyridine (as described in Example 54(a)) (55 mg, 0.21 mmol) and caesium carbonate (137 mg, 0.42 mmol) were suspended in dioxane (2 ml). Pd₂(dba)₃ (19 mg, 0.021 mmol) and Xantphos (20 mg, 0.035 mmol) were added and the mixture was stirred at 90° C. under argon atmosphere for 16 h. The mixture was diluted with DCM and filtered through diatomeous earth. The organic phase was washed with brine and concentrated. The residue was dissolved in DMSO and purified by preparative HPLC. The fractions containing the product were pooled and concentrated. Aqueous sodium bicarbonate was added and the mixture was extracted with DCM (×4). The organic phase was dried (MgSO₄), filtered and concentrated. The residue was dissolved in DCM (1 ml) and 1 M hydrochloric acid in ether (0.2 ml) was added. The solvent was evaporated. The residue was dissolved in DCM and methanol and the solvent was evaporated to give the title compound (66 mg, 53%).

¹H NMR (400 MHz, DMSO-d₆) δ ppm 10.36 (s, 1H) 8.89 (d, 1H) 8.74 (d, 1H) 8.35 (d, 1H) 7.59 (d, 1H) 4.76-4.86 (m, 1H) 3.83 (dd, 2H) 3.25 (t, 2H) 3.05-3.11 (m, 2H) 2.08-2.20 (m, 2H) 1.85-1.93 (m, 2H) 1.40-1.66 (m, 6H). MS (ESI) m/z 554; 556 (M+1).

### Example 58

### {5-Fluoro-4-[2-methyl-3-(tetrahydro-pyran-4-yl)-3H-imidazol-4-yl]-pyrimidin-2-yl}-[6-(4-methyl-piperazine-1-sulfonyl)-pyridin-3-yl]-amine

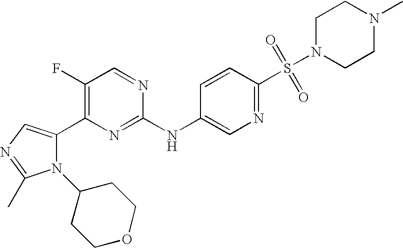

### Example 58(a)

### 1-(5-Bromo-pyridine-2-sulfonyl)-4-methyl-piperazine

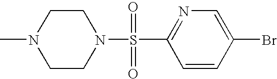

5-Bromopyridine-2-sulfonyl chloride (as described in Example 52(b) (55.0 mg, 0.214 mmol) was dissolved in CH₂Cl₂ (1 mL) and 1-methyl-piperazine (26 μL, 0.236 mmol) was added. Stirring was continued at room temperature for 3 hours and saturated aqueous NaHCO₃ (1 mL) was added. The mixture was diluted with CH₂Cl₂ (5 mL) and the aqueous phase was extracted with CH₂Cl₂ (3×5 mL). The combined organic phases were dried (Na₂SO₄) and concentrated to afford the title compound (61 mg, 89%).

¹H NMR (400 MHz, CDCl₃) δ ppm 2.29 (s, 3H) 2.33-2.38 (m, 2H) 2.45-2.50 (m, 2H) 3.10-3.18 (m, 2H) 3.30-3.37 (m, 2H) 7.31 (d, J=4.29 Hz, 1H) 7.81 (d, J=7.58 Hz, 1H) 8.04 (dd, J=8.34, 2.27 Hz, 1H); MS (ESI) m/z 321 (M+1).

### Example 58(b)

### {5-Fluoro-4-[2-methyl-3-(tetrahydro-pyran-4-yl)-3H-imidazol-4-yl]-pyrimidin-2-yl}-[6-(4-methyl-piperazine-1-sulfonyl)-pyridin-3-yl]-amine

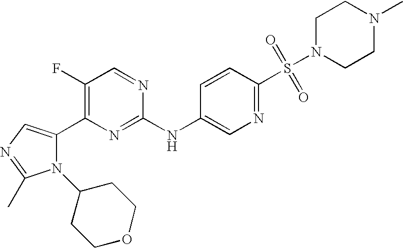

5-Fluoro-4-[2-methyl-1-(tetrahydro-2H-pyran-4-yl)-1H-imidazol-5-yl]pyrimidin-2-amine (as described in Example 6) (56.0 mg, 0.202 mmol), 1-(5-bromo-pyridine-2-sulfonyl)-4-methyl-piperazine (as described in Example 58(a)) (64.7 mg, 0.202 mmol), Cs₂CO₃ (131.7 mg, 0.404 mmol), Pd₂(dba)₃ (9 mg, 0.010 mmol) and XantPhos (12 mg, 0.020) mmol) were weighed out in a 25 mL round-bottom flask and dioxane (5 mL) was added. The system was flushed with argon and then heated to 90° C. and stirred for 17 hours. Water (30 mL) was added and the mixture was extracted with CH₂Cl₂ (3×30 mL). Drying (Na₂SO₄), filtration and concentration afforded a crude material, which was purified by preparative HPLC to afford the title compound (18 mg, 17%).

¹H NMR (400 MHz, CDCl₃) δ ppm 1.83-1.92 (m, 2H) 2.31 (s, 3H) 2.46-2.62 (m, 6H) 2.65 (s, 3H) 3.28-3.44 (m, 6H) 4.12 (dd, J=11.62, 4.80 Hz, 2H) 4.97-5.09 (m, 1H) 7.70 (br. s., 1H) 7.83-7.90 (m, 2H) 8.32-8.39 (m, 2H) 8.77 (d, J=2.02 Hz, 1H); MS (ESI) m/z 518 (M+1).

### Pharmaceutical Formulations

According to one aspect of the present invention there is provided a pharmaceutical formulation comprising the compound of formula (J) as a free base or a pharmaceutically acceptable salt thereof, in an essentially pure and isolated form, for use in the prevention and/or treatment of conditions associated with glycogen synthase kinase-3.

The formulation used in accordance with the present invention may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration as an ointment, patch or cream, for rectal administration as a suppository and for local administration in a body cavity or in a bone cavity.

The formulation may be in a form suitable for oral administration, for example as a tablet, for parenteral injection as a sterile solution or suspension. In general the above formulation may be prepared in a conventional manner using pharmaceutically carriers or diluents.

Suitable daily doses of the compound of formula (J) as a free base and pharmaceutically acceptable salts thereof in the treatment of a mammal, including human, are approximately 0.01 to 250 mg/kg bodyweight at per oral administration and about 0.001 to 250 mg/kg bodyweight at parenteral administration. The typical daily dose of the active ingredients varies within a wide range and will depend on various factors such as the relevant indication, the route of administration, the age, weight and sex of the patient and may be determined by a physician.

The compound of formula (I) as a free base or a pharmaceutically acceptable salt thereof, in an essentially pure and isolated form, may be used on its own but will usually be administered in the form of a pharmaceutical formulation in which the active ingredient is in association with pharmaceutically acceptable diluents, excipients or inert carrier. Dependent on the mode of administration, the pharmaceutical formulation may comprise from 0.05 to 99% w (percent by weight), for example from 0.10 to 50% w, of active ingredient, all percentages by weight being based on total composition.

A diluent or carrier includes water, aqueous poly(ethylene glycol), magnesium carbonate, magnesium stearate, talc, a sugar (such as lactose), pectin, dextrin, starch, tragacanth, microcrystalline cellulose, methyl cellulose, sodium carboxymethyl cellulose or cocoa butter.

A formulation of the invention can be in a unit dosage form such as a tablet or an injectable solution. The tablet may additionally comprise a disintegrant and/or may be coated (for example with an enteric coating or coated with a coating agent such as hydroxypropyl methylcellulose).

The invention further provides a process for the preparation of a pharmaceutical formulation of the invention which comprises mixing of the compound of formula (I) or a pharmaceutically acceptable salt thereof, a hereinbefore defined, with pharmaceutically acceptable diluents, excipients or inert carriers.

An example of a pharmaceutical formulations of the invention is an injectable solution comprising the compound of formula (I) as a free base or a pharmaceutically acceptable salt thereof, as hereinbefore defined, and sterile water, and, if necessary, either a base sodium hydroxide or an acid hydrochloric acid to bring the pH of the final formulation to about pH in the range of about 4 to 6, particularly about 5, and optionally a surfactant to aid dissolution. A suitable base is sodium hydroxide. A suitable acid is hydrochloric acid.

A suitable pharmaceutically acceptable salt of the compound of formula (I) useful in accordance to the invention is, for example, an acid-addition salt, which is sufficiently basic, for example an inorganic or organic acid. In addition a suitable pharmaceutically acceptable salt of the compounds of the invention, which is sufficiently acidic, is an alkali metal salt, an alkaline earth metal salt or a salt with an organic base, which affords a physiologically-acceptable cation.

### Medical Uses

It has been found that the compounds of formula (I) defined in the present invention, are well suited for inhibiting glycogen synthase kinase-3 (GSK3). Accordingly, said compound of the present invention is expected to be useful in the prevention and/or treatment of conditions associated with glycogen synthase kinase-3 activity, i.e. the compounds may be used to produce an inhibitory effect of GSK3 in mammals, including human, in need of such prevention and/or treatment.

GSK3 is highly expressed in the central and peripheral nervous system and in other tissues. Thus, it is expected that compound of the invention is well suited for the prevention and/or treatment of conditions associated with glycogen synthase kinase-3 in the central and peripheral nervous system. In particular, the compound of the invention is expected to be suitable for prevention and/or treatment of conditions associated with cognitive disorders and predemented states, especially dementia, Alzheimer's Disease (AD), Cognitive Deficit in Schizophrenia (CDS), Mild Cognitive Impairment (MCI), Age-Associated Memory Impairment (AAMI), Age-Related Cognitive Decline (ARCD) and Cognitive Impairment No Dementia (CIND), diseases associated with neurofibrillar tangle pathologies, Frontotemporal dementia (FTD), Frontotemporal dementia Parkinson's Type (FTDP), progressive supranuclear palsy (PSP), Pick's Disease, Niemann-Pick's Disease, corticobasal degeneration (CBD), traumatic brain injury (TBI) and dementia pugilistica.

One embodiment of the invention relates to the prevention and/or treatment of Alzheimer's Disease, especially the use in the delay of the disease progression of Alzheimer's Disease.

Other conditions are selected from the group consisting of Down's syndrome, vascular dementia, Parkinson's Disease (PD), postencephelatic parkinsonism, dementia with Lewy bodies, HIV dementia, Huntington's Disease, amyotrophic lateral sclerosis (ALS), motor neuron diseases (MND, Creuztfeld-Jacob's disease and prion diseases.

Other conditions are selected from the group consisting of attention deficit disorder (ADD), attention deficit hyperactivity disorder (ADHD) and affective disorders, wherein the affective disorders are Bipolar Disorder including acute mania, bipolar depression, bipolar maintenance, major depressive disorders (MDD) including depression, major depression, mood stabilization, schizoaffective disorders including schizophrenia, and dysthymia.

Other conditions are selected from the group consisting of Type I diabetes, Type II diabetes, diabetic neuropathy, alopecia, inflammatory diseases and cancer.

One embodiment of the invention relates to the use of a compound of the formula (J), as defined in the present invention, in the prevention and/or treatment of bone-related disorders or conditions in mammals.

One aspect of the invention is directed to the use of a compound of the formula (J), as defined in the present invention to treat osteoporosis.

One aspect of the invention is directed to the use of a compound of the formula (I), as defined in the present invention to increase and promote bone formation in mammals.

One aspect of the invention is directed to the use of a compound of the formula (I), as defined in the present invention to increase bone mineral density in mammals.

Another aspect of the invention is directed to the use of a compound of the formula (I), as defined in the present invention to reduce the rate of fracture and/or increase the rate of fracture healing in mammals.

Another aspect of the invention is directed to the use of a compound of the formula (I), as defined in the present invention to increase cancellous bone formation and/or new bone formation in mammals.

Another aspect of the invention is directed to a method of prevention and/or treatment of bone-related disorders comprising administering to a mammal in need of such prevention and/or treatment, a therapeutically effective amount of a compound of the formula (I) as defined in the present invention.

Another aspect of the invention is directed to a method of prevention and/or treatment of osteoporosis comprising administering to a mammal in need of such prevention and/or treatment, a therapeutically effective amount of a compound of the formula (I) as defined in the present invention.

Another aspect of the invention is directed to a method of increasing bone formation comprising administering to a mammal in need of such treatment, a therapeutically effective amount of a compound of the formula (I) as defined in the present invention.

Another aspect of the invention is directed to a method of increasing bone mineral density comprising administering to a mammal in need of such treatment, a therapeutically effective amount of a compound of the formula (I) as defined in the present invention.

Another aspect of the invention is directed to a method of reducing the incidence of fracture comprising administering to a mammal in need of such treatment, a therapeutically effective amount of a compound of the formula (I) as defined in the present invention.

Another aspect of the invention is directed to a method of enhancing fracture healing comprising administering to a mammal in need of such treatment, a therapeutically effective amount of a compound of the formula (I) as defined in the present invention.

Another aspect of the invention is directed to said methods and wherein said mammal is a human.

Another aspect of the invention is directed to said methods and wherein said mammal is a vertebrate animal, preferably but not limited to bigger animals such as horses, camels, dromedars but not limited thereto.

The use of the GSK3 inhibitors, the compounds of formula (I) hereinbefore defined, in primary and secondary osteoporosis, where primary osteoporosis includes postmenopausal osteoporosis and senile osteoporosis in both men and women, and secondary osteoporosis includes cortison induced osteoporosis, as well as any other type of induced secondary osteoporosis, are included in the term osteoporosis. In addition to this, these GSK3 inhibitors may also be used in treatments of myeloma. These GSK3 inhibitors may be administered locally or systemically, in different formulation regimes, to treat these conditions.

The promotion and increasing of bone formation makes the compounds of the formula (I) hereinbefore defined, suitable to reducing the incidence of fracture, to reduce the rate of fracture and/or increase the rate of fracture healing, to increase cancellous bone formation and/or new bone formation in mammals.

The use to promote and increase new bone formation may be in connection with surgery. This invention can be used during surgery, where the treating surgeon will place the invention locally in an appropriate formulation, near the deficient bone and/or in the body cavity. The bone may for instance have been broken, and utilizing the invention as described and claimed herein will then be placed in or near the fracture during open fracture repair. In some instances bone pieces may be missing (e.g. after tumour removal or severe casualties), and utilizing the invention as described and claimed herein will then be placed near the site of constructive bone surgery.

The present invention relates also to the use of the compound of formula (I) as as defined in the present invention in the manufacture of a medicament for the prevention and/or treatment of conditions associated with glycogen synthase kinase-3.

The invention also provides for a method of treatment and/or prevention of conditions associated with glycogen synthase kinase-3 comprising administering to a mammal, including human in need of such treatment and/or prevention a therapeutically effective amount of the compound of formula (I) as as defined in the present invention.

The dose required for the therapeutic or preventive treatment of a particular disease will necessarily be varied depending on the host treated, the route of administration and the severity of the illness being treated.

For veterinary use the amounts of different components, the dosage form and the dose of the medicament may vary and will depend on various factors such as, for example the individual requirement of the animal treated.

In the context of the present specification, the term “therapy” also includes “prevention” unless there are specific indications to the contrary. The terms “therapeutic” and “therapeutically” should be construed accordingly.

In the context of the present specification, the term “disorder” also includes “condition” unless there are specific indications to the contrary.

### Non-Medical Use

In addition to their use in therapeutic medicine, the compounds of formula (I) as a free base or a pharmaceutically acceptable salt thereof, are also useful as pharmacological tools in the development and standardisation of in vitro and in vivo test systems for the evaluation of the effects of inhibitors of GSK3 related activity in laboratory animals such as cats, dogs, rabbits, monkeys, rats and mice, as part of the search for new therapeutics agents.

### Pharmacology

### Determination of ATP Competition in Scintillation Proximity GSK3β Assay.

### GSK3β Scintillation Proximity Assay.

The competition experiments were carried out in duplicate with 10 different concentrations of the inhibitors in clear-bottom microtiter plates (Wallac, Finland). A biotinylated peptide substrate, Biotin-Ala-Ala-Glu-Glu-Leu-Asp-Ser-Arg-Ala-Gly-Ser(PO₃H₂)-Pro-Gln-Leu (AstraZeneca, Lund), was added at a final concentration of 1 μM in an assay buffer containing 1 mU recombinant human GSK3β (Dundee University, UK), 12 mM morpholinepropanesulfonic acid (MOPS), pH 7.0, 0.3 mM EDTA, 0.01% β-mercaptoethanol, 0.004% Brij 35 (a natural detergent), 0.5% glycerol and 0.5 μg BSA/25 μl. The reaction was initiated by the addition of 0.04 μCi [γ-³³P]ATP (Amersham, UK) and unlabelled ATP at a final concentration of 1 μM and assay volume of 25 μl. After incubation for 20 minutes at room temperature, each reaction was terminated by the addition of 25 μl stop solution containing 5 mM EDTA, 50 μM ATP, 0.1% Triton X-100 and 0.25 mg streptavidin coated Scintillation Proximity Assay (SPA) beads (Amersham, UK). After 6 hours the radioactivity was determined in a liquid scintillation counter (1450 MicroBeta Trilux, Wallac). The inhibition curves were analysed by non-linear regression using GraphPad Prism, USA. The Kₘ value of ATP for GSK3β, used to calculate the inhibition constants (Kᵢ) of the various compounds, was 20 μM.

The following abbreviations have been used:

MOPS Morpholinepropanesulfonic acid
EDTA Ethylenediaminetetraacetic acid

### BSA Bovin Serum Albumin

### ATP Adenosine Triphosphate

### SPA Scintillation Proximity Assay

GSK3 Glycogen synthase kinase 3

### Results

Typical Kᵢ values for the compounds of the present invention are in the range of about 0.001 to about 10,000 nM. Other values for Kᵢ are in the range of about 0.001 to about 1000 nM. Further values for Kᵢ are in the range of about 0.001 nM to about 700 nM.

TABLE 1
Specimen results from assay.

| Example | |
|---|---|
| no | Kᵢ (nM) |
| 1 | 468 |
| 2 | 40 |
| 3 | 661 |
| 4 | 63 |
| 5 | 70 |
| 10 | 45 |
| 11 | 60 |
| 12 | 29 |
| 13 | 34 |
| 14 | 16 |
| 15 | 43 |
| 16 | 9 |
| 17 | 50 |
| 18 | 47 |
| 19 | 25 |
| 20 | 26 |
| 21 | 90 |
| 22 | 67 |
| 23 | 41 |
| 24 | 24 |
| 25 | 28 |
| 26 | 31 |
| 27 | 28 |
| 28 | 19 |
| 29 | 34 |
| 30 | 17 |
| 31 | 12 |
| 32 | 33 |
| 33 | 16 |
| 34 | 85 |
| 35 | 20 |
| 36 | 10 |
| 37 | 56 |
| 38 | 29 |
| 39 | 160 |
| 40 | 16 |
| 42 | 11 |
| 43 | 23 |
| 44 | 55 |
| 45 | 32 |
| 46 | 39 |
| 47 | 100 |
| 48 | 16 |
| 49 | 19 |
| 50 | 33 |
| 51 | 21 |
| 52 | 39 |
| 53 | 20 |
| 54 | 7 |
| 55 | 18 |
| 56 | 55 |
| 57 | 19 |
| 58 | 30 |

1-77. (canceled)

78. A compound of formula (I): 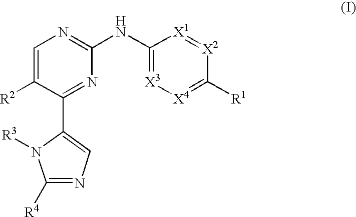
wherein:
R¹ is selected from sulphamoyl, carbamoyl, a group —R⁵-R⁶ and a nitrogen linked 4-7 membered saturated ring which optionally contains an additional nitrogen, oxygen or sulphur atom; wherein said ring is optionally substituted on carbon by one or more R⁷; and wherein if said ring contains an additional nitrogen atom that nitrogen is optionally substituted by R⁸;
at least one of X¹, X², X³ and X⁴ is selected from N, the other three X¹, X², X³ or X⁴ are independently selected from N or C(R⁹), provided that not more than two of X¹, X², X³ or X⁴ are selected from N;
R² is halo or cyano;
R³ is methyl, 3-tetrahydropyranyl or 4-tetrahydropyranyl, wherein the tetrahydropyranyl group is optionally substituted on carbon by one or more R¹⁰;
R⁴ is selected from hydrogen, halo, cyano and C₁₋₃alkyl, wherein C₁₋₃alkyl is optionally substituted with one or more halo;
R⁵ is selected from —O—, —C(O)—, —C(O)O—, —C(O)N(R¹¹)—, —S(O)ᵣ, and —SO₂N(R¹²)—; wherein R¹¹ and R¹² are independently selected from hydrogen or C₁₋₆alkyl and said alkyl is optionally substituted by one or more R¹³; and r is 0, 1 or 2;
R⁶ is selected from C₁₋₆alkyl, carbocyclyl and heterocyclyl; wherein R⁶ is optionally substituted on carbon by one or more R¹⁴; and wherein if said heterocyclyl contains an —NH— moiety that nitrogen is optionally substituted by a group selected from R¹⁵;
R⁷ is selected from halo, cyano, hydroxy, trifluoromethoxy, C₁₋₃alkoxy and C₁₋₃alkyl, wherein said C₁₋₃alkyl is optionally substituted by one or more halo;
R⁹ is selected from hydrogen, halo, cyano, hydroxy, amino, C₁₋₃alkyl and C₁₋₃alkoxy;
R¹⁰, R¹³ and R¹⁴ are independently selected from halo, cyano, hydroxy, amino, sulphamoyl, C₁₋₁₆alkyl, C₁₋₆alkoxy, C₁₋₆alkoxyC₁₋₆alkoxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, N—(C₁₋₆alkyl)sulphamoyl, N,N—(C₁₋₆alkyl)₂sulphamoyl, C₁₋₆alkylsulphonylamino, carbocyclyl, heterocyclyl, carbocyclylC₁₋₃alkyl-R¹⁶—, heterocyclylC₁₋₃alkyl-R¹⁷—, carbocyclyl-R¹⁸— and heterocyclyl-R¹⁹—; wherein R¹⁰, R¹³ and R¹⁴ are independently of each other substituted on carbon by one or more R²⁰; and wherein if said heterocyclyl contains an —NH— moiety that nitrogen is optionally substituted by a group selected from R²¹;
R¹⁶, R¹⁷, R¹⁸ and R¹⁹ are independently selected from —O—, —N(R²²)—, —C(O)—, —N(R²³)C(O)—, —C(O)N(R²⁴)—, —S(O)ₛ—, —SO₂N(R²⁵)— and —N(R²⁶)SO₂—; wherein R²², R²³, R²⁴, R²⁵ and R²⁶ are independently selected from hydrogen and C₁₋₆alkyl; and s is 0, 1 or 2;
R⁸, R¹⁵ and R²¹ are independently selected from C₁₋₄alkyl, carbocyclyl, heterocyclyl, —C₁₋₄alkylcarbocyclyl, —C₁₋₄alkylheterocyclyl, C₁₋₄alkanoyl, C₁₋₄alkylsulphonyl and C₁₋₄alkoxycarbonyl; wherein R⁸, R¹⁵ and R²¹ independently of each other may be optionally substituted on carbon by one or more R²⁷; and
R²⁰ and R²⁷ are independently selected from halo, cyano, hydroxy, trifluoromethoxy, trifluoromethyl, amino, methyl, ethyl, phenyl, cyclopropyl, cyclobutyl, methoxy, ethoxy, methylamino, ethylamino, dimethylamino, diethylamino, mesyl, ethylsulphonyl and phenyl;
as a free base or a pharmaceutically acceptable salt thereof.

79. A compound according to claim 78, wherein
R¹ is a group —R⁵-R⁶ or a nitrogen linked 4-7 membered saturated ring which optionally contains an additional nitrogen, oxygen or sulphur atom; wherein said ring may be optionally substituted on carbon by one or more R⁷; and wherein if said ring contains an additional nitrogen atom that nitrogen is optionally substituted by R⁸;
at least one of X¹, X², X³ and X⁴ is selected from N, the other three X¹, X², X³ or X⁴ are independently selected from N or C(R⁹) provided that not more than two of X¹, X², X³ or X⁴ are selected from N;
R² is halo or cyano;
R³ is methyl or 4-tetrahydropyranyl, wherein said tetrahydropyranyl group is optionally substituted on carbon by one or more R¹⁰;
R⁴ is selected from hydrogen, halo, cyano and C₁₋₃alkyl, wherein said C₁₋₃alkyl is optionally substituted with one or more halo;
R⁵ is selected from —O—, —C(O)—, —C(O)O—, —C(O)N(R¹¹)—, —S(O)ᵣ— and —SO₂N(R¹²)—; wherein R¹¹ and R¹² are independently selected from hydrogen or C₁₋₆alkyl and said alkyl is optionally substituted by one or more R¹³; and r is 0 or 2;
R⁶ is selected from C₁₋₆alkyl, carbocyclyl and heterocyclyl; wherein R⁶ is optionally substituted on carbon by one or more R⁴; and wherein if said heterocyclyl contains an —NH— moiety that nitrogen is optionally substituted by a group selected from R¹⁵;
R⁷ is selected from halo, cyano, hydroxy, trifluoromethoxy, C₁₋₃alkoxy and C₁₋₃alkyl, wherein said C₁₋₃alkyl is optionally substituted by one or more halo;
R⁹ is selected from hydrogen, halo, cyano, hydroxy, C₁₋₃alkyl and C₁₋₃alkoxy;
R¹⁰, R¹³ and R¹⁴ are independently selected from halo, cyano, hydroxy, amino, sulphamoyl, C₁₋₆alkyl, C₁₋₆alkoxy, C₁₋₆alkoxyC₁₋₆alkoxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, N—(C₁₋₆alkyl)sulphamoyl, N,N—C₁₋₆alkyl)₂sulphamoyl, C₁₋₆alkylsulphonylamino, carbocyclyl, heterocyclyl, carbocyclylC₁₋₃alkyl-R¹⁶—, heterocyclylC₁₋₃alkyl-R¹⁷—, carbocyclyl-R¹⁸— and heterocyclyl-R¹⁹—; wherein R¹⁰, R¹³ and R¹⁴ independently of each other are optionally substituted on carbon by one or more R²⁰; and wherein if said heterocyclyl contains an —NH— moiety that nitrogen is optionally substituted by a group selected from R²¹;
R¹⁶, R¹⁷, R¹⁸ and R¹⁹ are independently selected from —O—, —N(R²²)—, —C(O)—, —N(R²³)C(O)—, —C(O)N(R²⁴)—, —S(O)ₛ—, —SO₂N(R²⁵)— and —N(R²⁶)SO₂—; wherein R²², R²³, R²⁴, R²⁵ and R²⁶ are independently selected from hydrogen or C₁₋₆alkyl; and s is 0, 1 or 2;
R⁸, R¹⁵ and R²¹ are independently selected from C₁₋₄alkyl, carbocyclyl, heterocyclyl, —C₁₋₄alkylcarbocyclyl, —C₁₋₄alkylheterocyclyl, C₁₋₄alkanoyl, C₁₋₄alkylsulphonyl and C₁₋₄alkoxycarbonyl; wherein R⁸, R¹⁵ and R²¹ independently of each other may be optionally substituted on carbon by one or more R²⁷; and
R²⁰ and R²⁷ are independently selected from halo, cyano, hydroxy, trifluoromethoxy, trifluoromethyl, amino, methyl, ethyl, phenyl, cyclopropyl, cyclobutyl, methoxy, ethoxy, methylamino, ethylamino, dimethylamino, diethylamino, mesyl and ethylsulphonyl; as a free base or a pharmaceutically acceptable salt, an in vivo hydrolysable ester, solvate or solvate of a salt thereof.

80. A compound according to claim 78 or 79, wherein R² is halo.

81. A compound according to claim 78 or 79, wherein R² is fluoro.

82. A compound according to claim 78 or 79, wherein R³ is 4-tetrahydropyranyl or methyl.

83. A compound according to claim 78 or 79, wherein R⁴ is hydrogen or C₁₋₃alkyl, wherein said C₁₋₃alkyl is optionally substituted with one or more halo.

84. A compound according to claim 83, wherein R⁴ is C₁₋₃alkyl.

85. A compound according to claim 84, wherein R⁴ is methyl.

86. A compound according to claim 83, wherein R⁴ is trifluoromethyl.

87. A compound according to claim 78 or 79, wherein R⁵ is —C(O)— or —S(O)ᵣ—; and r is 0 or 2.

88. A compound according to claim 87, wherein R⁵ is —C(O)—.

89. A compound according to claim 87, wherein R⁵ is —S(O)ᵣ—; and r is 2.

90. A compound according to claim 78 or 79, wherein R⁵ is —O— or C(O)O—.

91. A compound according to claim 78 or 79, wherein R⁵ is —C(O)N(R¹¹)— or —SO₂N(R¹²)—; wherein R¹¹ and R¹² are independently selected from hydrogen or C₁₋₆alkyl.

92. A compound according to claim 78 or 79, wherein R⁶ is C₁₋₆alkyl or heterocyclyl; wherein R⁶ is optionally substituted on carbon by one or more R¹⁴; and wherein if said heterocyclyl contains an —NH— moiety that nitrogen is optionally substituted by a group selected from R¹⁵.

93. A compound according to claim 92, wherein said C₁₋₆alkyl is methyl, ethyl, butan-2-yl, butan-3-yl, propan-2-yl or tert-butyl.

94. A compound according to claim 92, wherein said heterocyclyl is selected from morpholinyl, homomorpholinyl, piperidinyl, pyrrolidinyl, azetidinyl, piperazinyl, homopiperidinyl and homopiperazinyl.

95. A compound according to claim 94, wherein said heterocyclyl is selected from piperidinyl, pyrrolidinyl, azetidinyl and piperazinyl.

96. A compound according to claim 92, wherein R¹⁴ is C₁₋₆alkoxy, halo, C₁₋₆alkyl, carbocyclyl, heterocyclyl and N,N—(C₁₋₆alkyl)₂amino; wherein R¹⁴ is optionally substituted on carbon by one or more R²⁰.

97. A compound according to claim 92, wherein R¹⁵ is C₁₋₄alkyl or carbocycle; wherein R¹⁵ is optionally substituted on carbon by one or more R²⁷.

98. A compound according to claim 78 or claim 79, wherein R⁸ is C₁₋₄alkyl, and wherein R⁸ may be optionally substituted on carbon by one or more R²⁷.

99. A compound according to claim 97 or claim 98, wherein R²⁷ is hydroxy, halo, ethoxy, methoxy or phenyl.

100. A compound according to claim 98, wherein R²⁷ is hydroxy, halo, ethoxy, methoxy or phenyl.

101. A compound according to claim 78 or claim 79, wherein at least one of X², X³ and X⁴ is selected from N, the other two X², X³ or X⁴ are independently selected from N or C(R⁹).

102. A compound according to claim 101, wherein X³ or X⁴ is N.

103. A compound according to claim 78 or claim 79, wherein R⁹ is hydrogen, methyl, trifluoromethyl, trifluoromethoxy or halo.

104. A compound according to claim 103, wherein R⁹ is hydrogen.

105. A compound according to claim 103, wherein one of R⁹ is halo.

106. A compound according to claim 105, wherein said halo is chloro.

107. A compound according to claim 78 or claim 79, wherein
R¹ is a group —R⁵-R⁶;
at least one of X¹, X², X³ and X⁴ is selected from N, the other three X¹, X², X³ or X⁴ are independently selected from N or C(R⁹), provided that not more than two of X¹, X², X³ or X⁴ are selected from N;
R² is halo;
R³ is methyl or 4-tetrahydropyranyl;
R⁴ is C₁₋₃alkyl, wherein said C₁₋₃alkyl is optionally substituted with one or more halo;
R⁵ is selected from —O—, —C(O)—, —C(O)O—, —C(O)N(R¹¹)—, —S(O)ᵣ— and —SO₂N(R¹²)—; wherein
R¹¹ and R¹² are independently selected from hydrogen or C₁₋₆alkyl and said alkyl is optionally substituted by one or more R¹³ and r is 2;
R⁶ is C₁₋₆alkyl or heterocyclyl; wherein R⁶ is optionally substituted on carbon by one or more R¹⁴; and wherein if said heterocyclyl contains an —NH— moiety that nitrogen is optionally substituted by a group selected from R¹⁵;
R⁹ is hydrogen or halo;
R¹⁴ is selected from halo, C₁₋₆alkyl, carbocycle, N,N—(C₁₋₆alkyl)₂amino, heterocyclyl and C₁₋₆alkoxy; wherein R¹⁴ is optionally on carbon by one or more R²⁰;
R¹⁵ is C₁₋₄alkyl or carbocycle; wherein R¹⁵ is optionally substituted on carbon by one or more R²⁷; and
R²⁰ and R²⁷ are independently selected from halo, methoxy, ethoxy, and phenyl.

108. A compound according to claim 78 or 79, wherein,
R¹ is a group —R⁵-R⁶;
at least one of X¹, X², X³ and X⁴ is selected from N, the other three X¹, X², X³ or X⁴ are independently selected from N or C(R⁹), provided that not more than two of X¹, X², X³ or X⁴ are selected from N;
R² is halo;
R³ is 4-tetrahydropyranyl;
R⁴ is C₁₋₃alkyl;
R⁵ is —C(O) or —S(O)ᵣ— and —SO₂N(R¹²)—; and r is 2;
R⁶ is C₁₋₆alkyl or heterocyclyl; wherein if said heterocyclyl contains an —NH— moiety that nitrogen is optionally substituted by a group selected from R¹⁵;
R⁹ is hydrogen; and
R¹⁵ is C₁₋₄alkyl.

109. A pharmaceutical formulation comprising as active ingredient a therapeutically effective amount of a compound according to in claim 78 or claim 79 in association with pharmaceutically acceptable excipients, carriers or diluents.

110. A method of prevention and/or treatment of conditions associated with glycogen synthase kinase-3, comprising administering to a human in need of such prevention and/or treatment a therapeutically effective amount of a compound as defined in claim 78 or claim 79.

111. A method of prevention and/or treatment of cognitive disorders, comprising administering to a human in need of such prevention and/or treatment a therapeutically effective amount of a compound as defined in claim 78 or claim 79.

112. The method according to claim 111, wherein the cognitive disorder is dementia, Cognitive Deficit in Schizophrenia (CDS), Mild Cognitive Impairment (MCI), Age-Associated Memory Impairment (AAMI), Age-Related Cognitive Decline (ARCD) or Cognitive Impairment No Dementia (CIND).

113. The method according to claim 112, wherein the disease is Cognitive Deficit in Schizophrenia.

114. The method according to claim 112, wherein the dementia is associated with neurofibrillar tangle pathologies.

115. The method according to claim 112, wherein the dementia is Frontotemporal dementia (FTD), Frontotemporal dementia Parkinson's Type (FTDP), progressive supranuclear palsy (PSP), Pick's Disease, Niemann-Pick's Disease, corticobasal degeneration, traumatic brain injury (TBI) or dementia pugilistica.

116. The method according to claim 115, wherein the dementia is Alzheimer's Disease (AD), Down syndrome, vascular dementia, Parkinson's Disease (PD), postencephelatic parkinsonism, dementia with Lewy bodies, HIV dementia, Huntington's Disease, amyotrophic lateral sclerosis (ALS), motor neuron diseases (MND), Creuztfeld-Jacob's disease or prion diseases.

117. The method according to claim 116, wherein the dementia is Alzheimer's Disease.

118. The method according to claim 116, wherein the treatment is in the delay of the disease progression of Alzheimer's Disease.

119. A method of prevention and/or treatment of attention deficit disorder (ADD), attention deficit hyperactivity disorder (ADHD) or affective disorders, comprising administering to a human in need of such prevention and/or treatment, a therapeutically effective amount of a compound as defined in claim 78 or claim 79.

120. The method according to claim 119, wherein the affective disorders are Bipolar Disorder including acute mania, bipolar depression, bipolar maintenance, major depressive disorders (MDD) including depression, major depression, mood stabilization, schizoaffective disorders including schizophrenia, or dysthymia.

121. A method of prevention and/or treatment of Type I diabetes, Type II diabetes, diabetic neuropathy, alopecia, inflammatory diseases or cancer, comprising administering to a human in need of such prevention and/or treatment a therapeutically effective amount of a salt compound as defined in claim 78 or claim 79.

122. A method of prevention and/or treatment of bone related disorders or conditions comprising administering to a human in need of such prevention and/or treatment a therapeutically effective amount of a salt compound as defined in claim 78 or claim 79.

123. A method of prevention and/or treatment of osteoporosis comprising administering to a human in need of such prevention and/or treatment a therapeutically effective amount of a compound as defined in claim 78 or claim 79.

124. A method of increasing bone formation comprising administering to a human in need of such prevention and/or treatment a therapeutically effective amount of a compound as defined in claim 78 or claim 79.

125. A method of increasing cancellous bone formation and/or new bone formation comprising administering to a human in need of such prevention and/or treatment a therapeutically effective amount of a compound as defined in claim 78 or claim 79.

126. A method of increasing bone mineral density comprising administering to a human in need of such prevention and/or treatment a therapeutically effective amount of a compound as defined in claim 78 or claim 79.

127. A method of reducing the incidence of fracture comprising administering to a human in need of such prevention and/or treatment a therapeutically effective amount of a compound as defined in claim 78 or claim 79.

128. A method of enhancing fracture healing comprising administering to a human in need of such prevention and/or treatment a therapeutically effective amount of a compound as defined in claim 78 or claim 79.

129. A process for preparing a compound of formula (I), or a pharmaceutically acceptable salt or an in vivo hydrolysable ester thereof, which process comprises:
a) reacting a pyrimidine of formula (II): 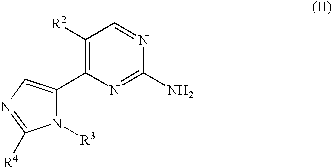
with a compound of formula (III): 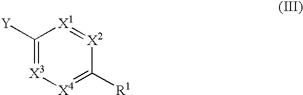
wherein
Y is a displaceable group; and
R¹, R², R³, R⁴, X¹, X², X³ and X⁴ are, unless otherwise specified, defined as in claim 78; and thereafter optionally:
b) converting a compound of the formula (I) into another compound of formula (I);
c) removing any protecting groups; and
d) forming a pharmaceutically acceptable salt or in vivo hydrolysable ester.
